(19) United States
(12) Patent Application Publication
(10) Pub. No.: US 2026/0317332 A1
(43) Pub. Date: Oct. 15, 2026

(54) BICYCLIC HETEROCYCLE COMPOUNDS FOR THE TREATMENT OF CANCER

(71) Applicant: Hoffmann-La Roche Inc., Little Falls, NJ (US)

(72) Inventors: Shuai CHEN, Shanghai (CN); Lan LI, Shanghai (CN); Yanhua LIU, Shanghai (CN); Ming LYU, Shanghai (CN); Hongtao XU, Shanghai (CN); Dan ZHAO, Shanghai (CN); Guanqun ZHENG, Shanghai (CN); Ge Zou, Shanghai (CN)

(73) Assignee: Hoffmann-La Roche Inc., Little Falls, NJ (US)

(21) Appl. No.: 19/478,443

(22) PCT Filed: Apr. 25, 2024
(86) PCT No.: PCT/EP2024/061335

§ 371 Date: Oct. 24, 2025

(30) Foreign Application Priority Data
Apr. 28, 2023 (WO) PCT/CN2023/091719
Sep. 19, 2023 (WO) PCT/CN2023/119806

(51) Int. Cl. C07D 487/04; A61K 31/513; A61K 31/53; A61K 31/538; C07B 59/00

(52) U.S. Cl. CPC C07D 487/04; A61K 31/513; A61K 31/53; A61K 31/538; C07B 59/002

(57) ABSTRACT

The present invention relates to compounds of formula (I), wherein R¹ to R⁵, T and L are as described herein, and their pharmaceutically acceptable salt thereof, and compositions including the compounds and methods of using the compounds.

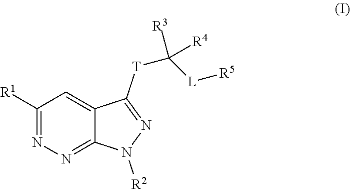

The present invention relates to organic compounds useful for therapy and/or prophylaxis in a mammal, and in particular to inhibition of CD73 useful for treating cancers.

### FIELD OF THE INVENTION

Hyper activation of the adenosine pathway contributes to immunosuppressive tumor microenvironment (TME) that impairs anti-tumor immunity and limits efficacy of immune checkpoint inhibitors. In the last step of the adenosine pathway, the enzyme ecto-5′-nucleotidase (CD73) catalyzes the conversion of AMP to adenosine, which is recognized by the adenosine receptors present in multiple immune cell-types, leading to suppression of the effector T cells and natural killer (NK) cells, activation of the regulatory T (Treg) and myeloid-derived suppressor cells (MDSCs), as well as other changes in the immune system that collectively culminate in an immunosuppressed environment. CD73 is frequently overexpressed in cancers and its upregulation is associated with poor clinical prognosis. Preclinical work in various in vivo tumor models demonstrated restoration of immune cell function and tumor growth inhibition upon genetic ablation or pharmacological inhibition of CD73. Therefore, it is conceivable that alleviating the immunosuppressive TME through CD73 inhibition has the therapeutic potential for restoring anti-tumor immunity and enhancing efficacy of immunotherapy to induce tumor regression. Given the uprising and unmet need for efficacious cancer treatments, inhibition of CD73 activity through administration of small molecule (SM) holds promise. This disclosure describes the invention of new small molecule CD73 inhibitors.

### SUMMARY OF THE INVENTION

The present invention relates to novel compounds of formula (I),

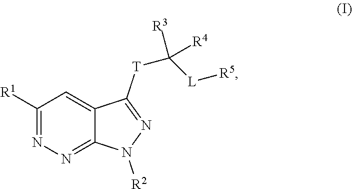

wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (C₁₋₆alkoxyC₃₋₇cycloalkyl)C₁₋₆alkoxy, (C₁₋₆alkyltetrahydrofuranyl)oxy, (cyanoC₃₋₇cycloalkyl)C₁₋₆alkoxy, (haloC₁₋₆alkoxy)C₁₋₆alkyl, (haloC₁₋₆alkylC₃₋₇cycloalkyl)C₁₋₆alkoxy, (haloC₃₋₇cycloalkyl)amino, (haloC₃₋₇cycloalkyl)C₁₋₆alkoxy, (halopyrrolidinyl)C₁₋₆alkyl, C₁₋₆alkoxy, C₁₋₆alkoxyC₁₋₆alkoxy, C₃₋₇cycloalkoxyC₁₋₆alkyl, cyanoC₁₋₆alkoxy, deuterioC₁₋₆alkoxy, deuteriohaloC₁₋₆alkoxy, haloC₁₋₆alkoxy, haloC₁₋₆alkyl, haloC₁₋₆alkylamino, haloC₃₋₇cycloalkoxy, haloC₃₋₇cycloalkyl or halopiperidyl;
T is O;
L is 1,4-benzoxazinylene,
   indazolylene,
   pyrimidinylene,
   phenylene being unsubstituted or substituted by halogen, or
   pyridylene being unsubstituted or substituted by halogen;
or a pharmaceutically acceptable salt thereof.

The compounds of formula (I) or (I-1) showed good CD73 inhibition. In another embodiment, the compounds of this invention showed superior cancer cell inhibition. In addition, the compounds of formula (I) or (I-1) also showed good or improved human hepatocyte stability, cytotoxicity, solubility and PK profiles.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes C₁₋₆alkyl-O—.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl or trifluoromethyl.

The term “deuterioC₁₋₆alkyl” denotes a C₁₋₆alkyl group wherein at least one of the hydrogen atoms of the C₁₋₆alkyl group has been replaced by deuterio.

The term “deuterioC₁₋₆alkoxy” denotes a C₁₋₆alkoxy group wherein at least one of the hydrogen atoms of the C₁₋₆alkyl group has been replaced by deuterio.

The term “haloC₁₋₆alkoxy” denotes haloC₁₋₆alkyl-O—.

The term “deuteriohaloC₁₋₆alkoxy” denotes a haloC₁₋₆alkoxy group wherein at least one of the hydrogen atoms of the haloC₁₋₆alkoxy group has been replaced by deuterio. Example of deuteriohaloC₁₋₆alkoxy includes 1,1-dideuterio-2,2,2-trifluoro-ethoxy.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “haloC₃₋₇cycloalkyl” denotes a C₃₋₇cycloalkyl group wherein at least one of the hydrogen atoms of the C₃₋₇cycloalkyl group has been replaced by same or different halogen atoms, particularly fluoro atoms. Examples of haloC₃₋₇cycloalkyl include 2,2-difluorocyclopropyl and 1-fluorocyclopropyl.

The term “C₃₋₇cycloalkoxy” denotes C₃₋₇cycloalkyl-O—.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Examples of heterocycles include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl, and 2,3,4a,5,7,7a-hexahydro-[1,4]dioxino[2,3-c]pyrrolyl.

The term “heterocyclylene” denotes a divalent heterocyclyl group.

The term “aryl” denotes a monovalent aromatic carbocyclic mono- or bicyclic ring system comprising 6 to 10 carbon ring atoms. Examples of aryl moieties include phenyl and naphthyl.

The term “arylene” denotes a divalent aryl group.

The term “heteroaryl” refers to any mono-, bi-, or tricyclic aromatic ring system containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13^{th} ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 5-6 membered monocyclic aromatic groups where one or more ring atoms is nitrogen, sulfur or oxygen. In one embodiment, heteroaryl includes 7-12 membered bicyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl, 1H-pyrazolo[3,4-d]pyrimidine, 1H-pyrazolo[3,4-d]pyridazine, imidazo[1,5-a]pyrazine, imidazo[5,1-f][1,2,4]triazine, [1,2,4]triazolo[4,3-a]pyrazine, 1H-pyrazolo[3,4-c]pyridazine, 1H-pyrazolo[3,4-b]pyridine, 1H-pyrazolo[4,3-d]pyrimidine, 1H-pyrazolo[3,4-c]pyridine, 1H-pyrazolo[4,3-c]pyridine and purinyl, as well as benzo-fused derivatives, for example benzoxazolyl, benzofuryl, benzothiazolyl, benzothiadiazolyl, benzotriazolyl, benzoimidazolyl, indazolyl and indolyl.

The term “heteroarylene” denotes a divalent heteroaryl group.

In particular embodiments, a heterocyclyl group or a heteroaryl group is attached at a carbon atom of the heterocyclyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

The term “indazolylene” denotes a divalent indazolyl group.

The term “pyridylene” denotes a divalent pyridyl group.

The term “pyrimidinylene” denotes a divalent pyrimidinyl group.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

In one embodiment, the skilled in the art can understand the keto-enol tautomerism may exist for certain structures as illustrated below:

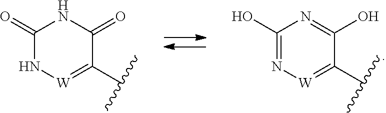

wherein W is CH or N.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment an optionally substituted group has 2 substituents. In another embodiment an optionally substituted group has 3 substituents. In another embodiment an optionally substituted group has 4 substituents. In another embodiment an optionally substituted group has 5 substituents.

The term “protecting group” or “PG” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of Cd73

The present invention relates to (i′) a compound of formula (I),

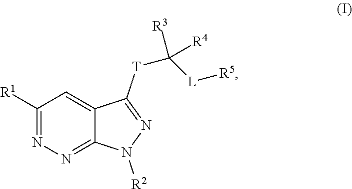

wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (C₁₋₆alkoxyC₃₋₇cycloalkyl)C₁₋₆alkoxy, (C₁₋₆alkyltetrahydrofuranyl)oxy, (cyanoC₃₋₇cycloalkyl)C₁₋₆alkoxy, (haloC₁₋₆alkoxy)C₁₋₆alkyl, (haloC₁₋₆alkylC₃₋₇cycloalkyl)C₁₋₆alkoxy, (haloC₃₋₇cycloalkyl)amino, (haloC₃₋₇cycloalkyl)C₁₋₆alkoxy, (halopyrrolidinyl)C₁₋₆alkyl, C₁₋₆alkoxy, C₁₋₆alkoxyC₁₋₆alkoxy, C₃₋₇cycloalkoxyC₁₋₆alkyl, cyanoC₁₋₆alkoxy, deuterioC₁₋₆alkoxy, deuteriohaloC₁₋₆alkoxy, haloC₁₋₆alkoxy, haloC₁₋₆alkyl, haloC₁₋₆alkylamino, haloC₃₋₇cycloalkoxy, haloC₃₋₇cycloalkyl or halopiperidyl;
T is O;
L is 1,4-benzoxazinylene,
   indazolylene,
   pyrimidinylene,
   phenylene being unsubstituted or substituted by halogen, or
   pyridylene being unsubstituted or substituted by halogen;
or a pharmaceutically acceptable salt thereof.

Another embodiment of present invention is (ii′) a compound of formula (I-1) according to (i′),

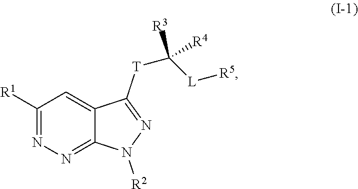

wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (C₁₋₆alkoxyC₃₋₇cycloalkyl)C₁₋₆alkoxy, (C₁₋₆alkyltetrahydrofuranyl)oxy, (cyanoC₃₋₇cycloalkyl)C₁₋₆alkoxy, (haloC₁₋₆alkoxy)C₁₋₆alkyl, (haloC₁₋₆alkylC₃₋₇cycloalkyl)C₁₋₆alkoxy, (haloC₃₋₇cycloalkyl)amino, (haloC₃₋₇cycloalkyl)C₁₋₆alkoxy, (halopyrrolidinyl)C₁₋₆alkyl, C₁₋₆alkoxy, C₁₋₆alkoxyC₁₋₆alkoxy, C₃₋₇cycloalkoxyC₁₋₆alkyl, cyanoC₁₋₆alkoxy, deuterioC₁₋₆alkoxy, deuteriohaloC₁₋₆alkoxy, haloC₁₋₆alkoxy, haloC₁₋₆alkyl, haloC₁₋₆alkylamino, haloC₃₋₇cycloalkoxy, haloC₃₋₇cycloalkyl or halopiperidyl;
T is O;
L is 1,4-benzoxazinylene,
   indazolylene,
   pyrimidinylene,
   phenylene being unsubstituted or substituted by halogen, or
   pyridylene being unsubstituted or substituted by halogen;
or a pharmaceutically acceptable salt thereof.

A further embodiment of present invention is (iii′) a compound of formula (I) or (I-1) according to (i′) or (ii′), or a pharmaceutically acceptable salt thereof, wherein R¹ is

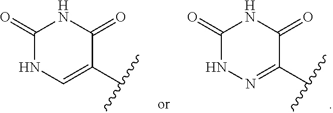 [SMILES: *c1c[nH]c(=O)[nH]c1=O ; *c1n[nH]c(=O)[nH]c1=O]

A further embodiment of present invention is (iv′) compound of formula (I) or (I-1) according to any one of (i′) to (iii′), or a pharmaceutically acceptable salt thereof, wherein R² is C₁₋₆alkyl.

A further embodiment of present invention is (v′) compound of formula (I) or (I-1) according to any one of (i′) to (iv′), or a pharmaceutically acceptable salt thereof, wherein R² is methyl.

A further embodiment of present invention is (vi′) a compound of formula (I) or (I-1) according to any one of (i′) to (v′), or a pharmaceutically acceptable salt thereof, wherein R³ is haloC₁₋₆alkyl.

A further embodiment of present invention is (vii′) a compound of formula (I) or (I-1) according to any one of (i′) to (vi′), or a pharmaceutically acceptable salt thereof, wherein R³ is difluoromethyl.

A further embodiment of present invention is (viii′) a compound of formula (I) or (I-1) according to any one of (i′) to (vii′), wherein L is pyrimidinylene, or pyridylene being unsubstituted or substituted by halogen.

A further embodiment of present invention is (ix′) a compound of formula (I) or (I-1) or a pharmaceutically acceptable salt thereof, according to any one of (i′) to (viii′), wherein L is pyrimidinylene, or pyridylene being unsubstituted or substituted by fluoro.

A further embodiment of present invention is (x′) a compound of formula (I) or (I-1) or a pharmaceutically acceptable salt thereof, according to any one of (i′) to (ix′), wherein R⁵ is (haloC₁₋₆alkoxy)C₁₋₆alkyl, (halopyrrolidinyl)C₁₋₆alkyl, C₃₋₇cycloalkoxyC₁₋₆alkyl, haloC₁₋₆alkoxy, haloC₁₋₆alkylamino, haloC₃₋₇cycloalkoxy or haloC₃₋₇cycloalkyl.

A further embodiment of present invention is (xi′) a compound of formula (I) or (I-1) or a pharmaceutically acceptable salt thereof, according to any one of (i′) to (x′), wherein R⁵ is (3,3-difluoropyrrolidin-1-yl)methyl, [2,2,2-trifluoro-1-methyl-ethyl]amino, 2,2,2-trifluoroethoxy, 2,2,2-trifluoroethoxymethyl, 2,2-difluoroethoxy, 3,3-difluorocyclobutoxy, 3,3-difluorocyclobutyl or cyclopropoxymethyl.

A further embodiment of present invention is (xii) a compound of formula (I) or (I-1), according to (i′) or (ii′), wherein
R¹ is

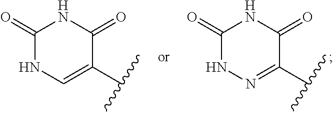 [SMILES: *c1c[nH]c(=O)[nH]c1=O ; *c1n[nH]c(=O)[nH]c1=O]

R² is C₁₋₆alkyl;
R³ is haloC₁₋₆alkyl;
R⁴ is H;
R⁵ is (haloC₁₋₆alkoxy)C₁₋₆alkyl, (halopyrrolidinyl)C₁₋₆alkyl, C₃₋₇cycloalkoxyC₁₋₆alkyl, haloC₁₋₆alkoxy, haloC₁₋₆alkylamino, haloC₃₋₇cycloalkoxy or haloC₃₋₇cycloalkyl;
T is O;
L is pyrimidinylene, or pyridylene being unsubstituted or substituted by halogen;
or a pharmaceutically acceptable salt thereof.

A further embodiment of present invention is (xiii′) a compound of formula (I) or (I-1), according to (xii′), wherein
R¹ is

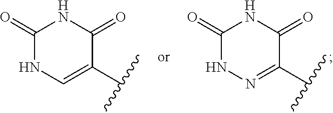 [SMILES: *c1c[nH]c(=O)[nH]c1=O ; *c1n[nH]c(=O)[nH]c1=O]

R² is methyl;
R³ is difluoromethyl;
R⁴ is H;
R⁵ is (3,3-difluoropyrrolidin-1-yl)methyl, [2,2,2-trifluoro-1-methyl-ethyl]amino, 2,2,2-trifluoroethoxy, 2,2,2-trifluoroethoxymethyl, 2,2-difluoroethoxy, 3,3-difluorocyclobutoxy, 3,3-difluorocyclobutyl or cyclopropoxymethyl;
T is O;
L is pyrimidinylene, or pyridylene being unsubstituted or substituted by fluoro;
or a pharmaceutically acceptable salt thereof.

The present invention relates to (i) a compound of formula (I),

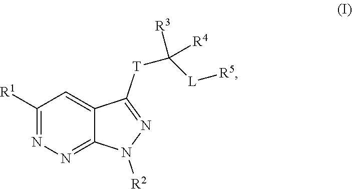

wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (cyanoC₃₋₇cycloalkyl)C₁₋₆alkoxy, (haloC₁₋₆alkylC₃₋₇cycloalkyl)C₁₋₆alkoxy, (haloC₃₋₇cycloalkyl)C₁₋₆alkoxy, C₁₋₆alkoxy, C₁₋₆alkoxyC₁₋₆alkoxy, C₃₋₇cycloalkoxyC₁₋₆alkyl, cyanoC₁₋₆alkoxy, deuteriohaloC₁₋₆alkoxy, haloC₁₋₆alkoxy or haloC₁₋₆alkyl;
T is O;
L is 1,4-benzoxazinylene, indazolylene, pyrimidinylene, phenylene being unsubstituted or substituted by halogen, or pyridylene being unsubstituted or substituted by halogen;
or a pharmaceutically acceptable salt thereof.

Another embodiment of present invention is (ii) a compound of formula (I-1) according to (i),

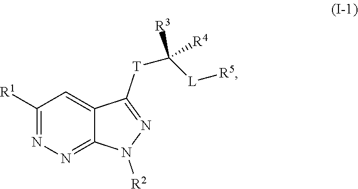

wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (cyanoC₃₋₇cycloalkyl)C₁₋₆alkoxy, (haloC₁₋₆alkylC₃₋₇cycloalkyl)C₁₋₆alkoxy, (haloC₃₋₇cycloalkyl)C₁₋₆alkoxy, C₁₋₆alkoxy, C₁₋₆alkoxyC₁₋₆alkoxy, C₃₋₇cycloalkoxyC₁₋₆alkyl, cyanoC₁₋₆alkoxy, deuteriohaloC₁₋₆alkoxy, haloC₁₋₆alkoxy or haloC₁₋₆alkyl;
T is O;
L is 1,4-benzoxazinylene, indazolylene, phenylene being unsubstituted or substituted by halogen, pyridylene being unsubstituted or substituted by halogen or pyrimidinylene;
or a pharmaceutically acceptable salt thereof.

A further embodiment of present invention is (iii) a compound of formula (I) or (I-1) according to (i) or (ii), or a pharmaceutically acceptable salt thereof, wherein R¹ is

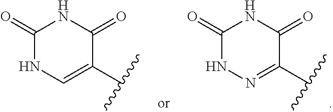 [SMILES: *c1c[nH]c(=O)[nH]c1=O ; *c1n[nH]c(=O)[nH]c1=O]

A further embodiment of present invention is (iv) compound of formula (I) or (I-1) according to any one of (i) to (iii), or a pharmaceutically acceptable salt thereof, wherein R² is methyl.

A further embodiment of present invention is (v) a compound of formula (I) or (I-1) according to any one of (i) to (iv), or a pharmaceutically acceptable salt thereof, wherein R³ is haloC₁₋₆alkyl.

A further embodiment of present invention is (vi) a compound of formula (I) or (I-1) according to any one of (i) to (v), or a pharmaceutically acceptable salt thereof, wherein R³ is difluoromethyl.

A further embodiment of present invention is (vii) a compound of formula (I) or (I-1) according to any one of (i) to (vi), wherein L is pyridylene being unsubstituted or substituted by halogen.

A further embodiment of present invention is (viii) a compound of formula (I) or (I-1) or a pharmaceutically acceptable salt thereof, according to any one of (i) to (vii), wherein L is pyridylene being unsubstituted or substituted by fluoro.

A further embodiment of present invention is (ix) a compound of formula (I) or (I-1) or a pharmaceutically acceptable salt thereof, according to any one of (i) to (viii), wherein R⁵ is C₃₋₇cycloalkoxyC₁₋₆alkyl or haloC₁₋₆alkoxy.

A further embodiment of present invention is (x) a compound of formula (I) or (I-1) or a pharmaceutically acceptable salt thereof, according to any one of (i) to (ix), wherein R⁵ is 2,2,2-trifluoroethoxy, 2,2-difluoroethoxy or cyclopropoxymethyl.

A further embodiment of present invention is (xi) a compound of formula (I) or (I-1), according to (i) or (ii), wherein
R¹ is

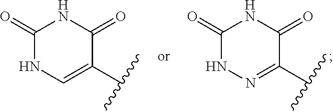 [SMILES: *c1c[nH]c(=O)[nH]c1=O ; *c1n[nH]c(=O)[nH]c1=O]

R² is C₁₋₆alkyl;
R³ is haloC₁₋₆alkyl;
R⁴ is H;
R⁵ is C₃₋₇cycloalkoxyC₁₋₆alkyl or haloC₁₋₆alkoxy;
T is O;
L is pyridylene being unsubstituted or substituted by halogen;
or a pharmaceutically acceptable salt thereof.

A further embodiment of present invention is (xii) a compound of formula (I) or (I-1), according to (xi), wherein
R¹ is

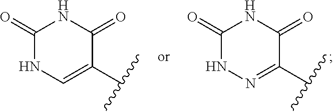 [SMILES: *c1c[nH]c(=O)[nH]c1=O ; *c1n[nH]c(=O)[nH]c1=O]

R² is methyl;
R³ is difluoromethyl;
R⁴ is H;
R⁵ is 2,2,2-trifluoroethoxy, 2,2-difluoroethoxy or cyclopropoxymethyl;
T is O;
L is pyridylene being unsubstituted or substituted by fluoro;
or a pharmaceutically acceptable salt thereof.

Another embodiment of present invention is (xiii) a compound selected from the following:
5-[3-[(1R)-2,2-difluoro-1-[3-(2,2,2-trifluoroethoxy)phenyl]ethoxy]-1-methyl-pyrazolo[3,4-c]pyridazin-5-yl]-1H-pyrimidine-2,4-dione;
5-[3-[(1R)-1-[2-(cyclopropoxymethyl)-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[4-fluoro-3-(2,2,2-trifluoroethoxy)phenyl]ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethoxy)-2-pyridyl]ethoxy]-1-(trideuteriomethyl)pyrazolo[3,4-c]pyridazin-5-yl]-1H-pyrimidine-2,4-dione;
5-[3-[(1R)-1-[4-(2,2-difluoroethoxy)-2-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione;
5-[3-[(1S)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione;
5-[3-[(1R)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1S)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1R)-2,2-difluoro-1-[2-(2,2,2-trifluoroethoxy)-4-pyridyl]ethoxy]-1-methyl-pyrazolo[3,4-c]pyridazin-5-yl]-1H-pyrimidine-2,4-dione;
5-[3-[(1S)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione;
6-[3-[(1R)-2,2-difluoro-1-[2-(2,2,2-trifluoroethoxy)-4-pyridyl]ethoxy]-1-methyl-pyrazolo[3,4-c]pyridazin-5-yl]-2H-1,2,4-triazine-3,5-dione;
6-[3-[(1R)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione;
6-[3-[(1S)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione;
5-[1-methyl-3-[(1S)-1-[6-(2,2,2-trifluoroethoxy)pyrimidin-4-yl]ethoxy]pyrazolo[3,4-c]pyridazin-5-yl]-1H-pyrimidine-2,4-dione;
5-[1-methyl-3-[(1R)-1-[6-(2,2,2-trifluoroethoxy)pyrimidin-4-yl]ethoxy]pyrazolo[3,4-c]pyridazin-5-yl]-1H-pyrimidine-2,4-dione;
5-[3-[(1R)-2,2-difluoro-1-[4-fluoro-3-(2-methoxyethoxy)phenyl]ethoxy]-1-methyl-pyrazolo[3,4-c]pyridazin-5-yl]-1H-pyrimidine-2,4-dione;
5-[3-[(1R)-1-[2-(2,2-difluoropropoxy)-4-pyridyl]-2,2-difluoro-ethoxy]-1-methyl-pyrazolo[3,4-c]pyridazin-5-yl]-1H-pyrimidine-2,4-dione;
5-[1-methyl-3-[(1R)-2,2-difluoro-1-[4-(2,2,2-trifluoro-1-methyl-ethoxy)-2-pyridyl]ethoxy]pyrazolo[3,4-c]pyridazin-5-yl]-1H-pyrimidine-2,4-dione;
5-[1-methyl-3-[(1R)-1-[4-[(2,2-difluorocyclopropyl)methoxy]-2-pyridyl]-2,2-difluoro-ethoxy]pyrazolo[3,4-c]pyridazin-5-yl]-1H-pyrimidine-2,4-dione;
5-[3-[(1R)-2,2-difluoro-1-[4-[(1-fluorocyclopropyl)methoxy]-2-pyridyl]ethoxy]-1-methyl-pyrazolo[3,4-c]pyridazin-5-yl]-1H-pyrimidine-2,4-dione;
5-[3-[(1R)-1-(4-ethoxy-2-pyridyl)-2,2-difluoro-ethoxy]-1-methyl-pyrazolo[3,4-c]pyridazin-5-yl]-1H-pyrimidine-2,4-dione; 2-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxy]acetonitrile; 1-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxymethyl]cyclopropanecarbonitrile;
5-[3-[(1R)-2,2-difluoro-1-[4-[[1-(trifluoromethyl)cyclopropyl]methoxy]-2-pyridyl]ethoxy]-1-methyl-pyrazolo[3,4-c]pyridazin-5-yl]-1H-pyrimidine-2,4-dione;
5-[3-[(1R)-2,2-difluoro-1-[1-(2,2,2-trifluoroethyl)indazol-6-yl]ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethyl)-2,3-dihydro-1,4-benzoxazin-6-yl]ethoxy]-1-methyl-pyrazolo[3,4-c]pyridazin-5-yl]-1H-pyrimidine-2,4-dione;
5-[3-[(1R)-2,2-difluoro-1-[2-[[(1S)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione;
5-[3-[(1R)-2,2-difluoro-1-[2-[[(1R)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione;
5-[3-[(1R)-2,2-difluoro-1-[2-(2-methoxyethoxy)-4-pyridyl]ethoxy]-1-methyl-pyrazolo[3,4-c]pyridazin-5-yl]-1H-pyrimidine-2,4-dione;
5-[3-[(1R)-1-(2-ethoxy-4-pyridyl)-2,2-difluoro-ethoxy]-1-methyl-pyrazolo[3,4-c]pyridazin-5-yl]-1H-pyrimidine-2,4-dione;
5-[3-[(1R)-2,2-difluoro-1-[2-(1,1,2,2,2-pentadeuterioethoxy)-4-pyridyl]ethoxy]-1-methyl-pyrazolo[3,4-c]pyridazin-5-yl]-1H-pyrimidine-2,4-dione;
5-[3-[(1R)-1-[2-(4,4-difluoro-1-piperidyl)-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(4,4-difluorocyclohexyl)amino]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(3,3-difluoropyrrolidin-1-yl)methyl]-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[2-(2,2,2-trifluoroethoxymethyl)-4-pyridyl]ethoxy]-1-methyl-pyrazolo[3,4-c]pyridazin-5-yl]-1H-pyrimidine-2,4-dione;
5-[3-[(1R)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione;
5-[3-[(1S)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione;
5-[3-[(1R)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione;
5-[3-[(1S)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione;
5-[3-[(1R)-1-[2-[(1S)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(1R)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-(3,3-difluorocyclobutyl)-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione;
5-[3-[(1S)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione;
5-[1-methyl-3-[(1R)-2,2-difluoro-1-[2-[(trans)-4-methyltetrahydrofuran-3-yl]oxy-4-pyridyl]ethoxy]pyrazolo[3,4-c]pyridazin-5-yl]-1H-pyrimidine-2,4-dione;
5-[3-[(1R)-2,2-difluoro-1-[2-1[(1-methoxycyclopropyl)methoxy]-4-pyridyl]ethoxy]-1-methyl-pyrazolo[3,4-c]pyridazin-5-yl]-1H-pyrimidine-2,4-dione;
5-[3-[(1R)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione; and
5-[3-[(1S)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione;
   or a pharmaceutically acceptable salt thereof.

Another embodiment of present invention is related to (xv′) a process for the preparation of a compound according to any one of (i) to (xii) or (i′) to (xiii′) comprising any one of the following steps:
a) Deprotection of compound of formula (X),

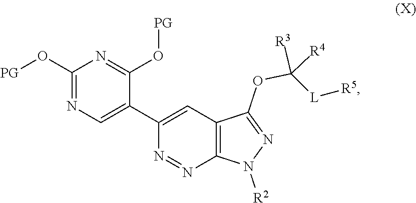

in the presence of an acid, dealkylation reagent, or metal mediated hydrogenation to afford the compound of formula (Ia),

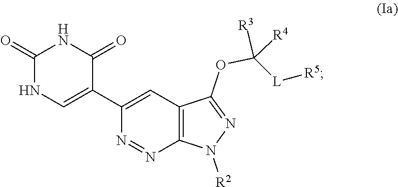

b) Deprotection of compound of formula (XIII),

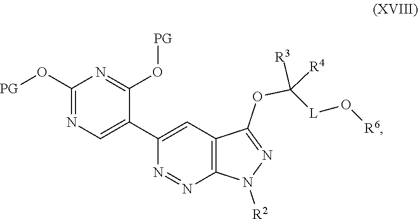

in the presence of an acid, dealkylation reagent, or metal mediated hydrogenation to afford the compound of formula (Ib),

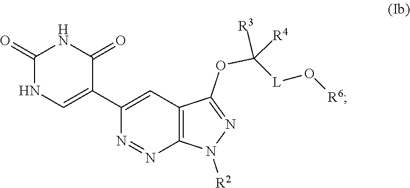

c) Stille coupling of compound of formula (XV),

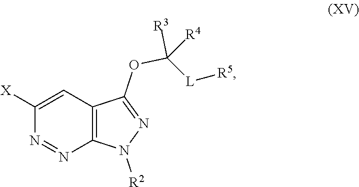

with organostannane (XVI),

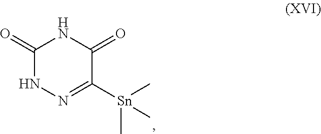

in the presence of a palladium catalyst to afford the compound of formula (Ic),

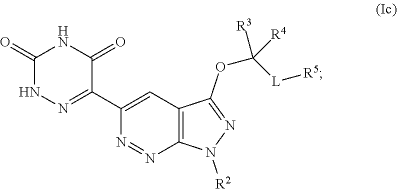

wherein
X is halogen; each PG is independently an oxygen protecting group; wherein PG is selected from methyl, tert-butyl, TBS, ethoxymethyl and benzyl; R⁶ is optionally substituted aryl, heteroaryl, heterocyclyl, arylC₁₋₆alkyl, heterocyclylC₁₋₆alkyl or heteroarylC₁₋₆alkyl;
in step a) and b), the acid is trifluoroacetic acid or aqueous hydrochloric acid; the dealkylation reagent is TMSCl and NaI; the hydrogenation is conducted with Pd/C;
in step c), the catalyst is Pd(PPh₃)₄;
R¹ to R⁵ and L are defined as in any one of (i) to (xii) or (i′) to (xiii′).

Another embodiment of present invention is (xvi′) a compound or pharmaceutically acceptable salt according to any one of (i) to (xii) or (i′) to (xiv′) for use as therapeutically active substance.

Another embodiment of present invention is (xvii′) a pharmaceutical composition comprising a compound in accordance with any one of (i) to (xii) or (i′) to (xiv′) and a pharmaceutically acceptable excipient.

Another embodiment of present invention is (xviii′) the use of a compound according to any one of (i) to (xii) or (i′) to (xiv′) for treating cancers.

Another embodiment of present invention is (xix′) the use according to (xvii), wherein the cancer is pancreatic cancer, colorectal cancer, gastric cancer, esophageal cancer, liver cancer, lung cancer, breast cancer, ovarian cancer, prostate cancer or melanoma.

Another embodiment of present invention is (xx′) the use of a compound according to any one of (i) to (xii) or (i′) to (xiv′) for inhibiting CD73.

Another embodiment of present invention is (xxi′) the use of a compound according to any one of (i) to (xii) or (i′) to (xiv′) for the preparation of a medicament for the treatment or prophylaxis of cancers, wherein the cancer is pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia.

Another embodiment of present invention is (xxii′) the use of a compound according to any one of (i) to (xii) or (i′) to (xiv′) for the preparation of a medicament as a CD73 inhibitor.

Another embodiment of present invention is (xxiii′) a compound or pharmaceutically acceptable salt according to any one of (i) to (xii) or (i′) to (xiv′), when manufactured according to a process of (xiv).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit the enzymatic activity of CD73 protein in converting AMP to adenosine. In one example, the pharmaceutically effective amount of the compound of the invention administered parenterally per dose will be in the range of about 0.01 to 100 mg/kg, alternatively about 0.1 to 50 mg/kg of patient body weight per day, with the typical initial range of compound used being 0.3 to 30 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 0.1 mg to 500 mg of the compound of the invention compounded with about 0.1 to 500 mg anhydrous lactose, about 0.1 to 500 mg sodium croscarmellose, about 0.1 to 500 mg polyvinylpyrrolidone (PVP) K30, and about 0.1 to 500 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 1 to 450 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of cancers. Another embodiment includes a pharmaceutical composition comprising a compound of formula (I) for use in the treatment of cancer.

The following embodiments illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention inhibit the enzymatic activity of CD73 in converting AMP to adenosine. Accordingly, the compounds of the invention are useful for reducing the adenosine levels in the TME. Compounds of the invention are useful for promoting immune-mediated killing of cancer cells that overexpress CD73, e.g. pancreatic cancer, colorectal cancer, gastric cancer, esophageal cancer, head and neck cancer, liver cancer, lung cancer, breast cancer, ovarian cancer, prostate cancer, melanoma, multiple myeloma, acute myeloid leukemia, or acute and chronic lymphoblastic leukemia. Alternatively, compounds of the invention are useful for promoting immune-mediated killing of cancer cells that are dependent on the adenosine pathway or in malignant solid tumor where the adenosine pathway is potentiated by dysregulation or mutation of effector pathways as EGFR-RAS-MAPK, PI3K-AKT-driven signaling, for targeted therapy in pancreatic adenocarcinoma, non-small cell lung cancer, esophageal and gastric adenocarcinoma, etc. More broadly, the compounds can be used for the treatment and prophylaxis of all cancer types which exhibit immunosuppressive TME.

Another embodiment includes a method of treating or preventing cancer in a mammal in need of such treatment, wherein the method comprises administering to said mammal a therapeutically effective amount of a compound of formula (I), a stereoisomer, tautomer or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁵, T and L are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

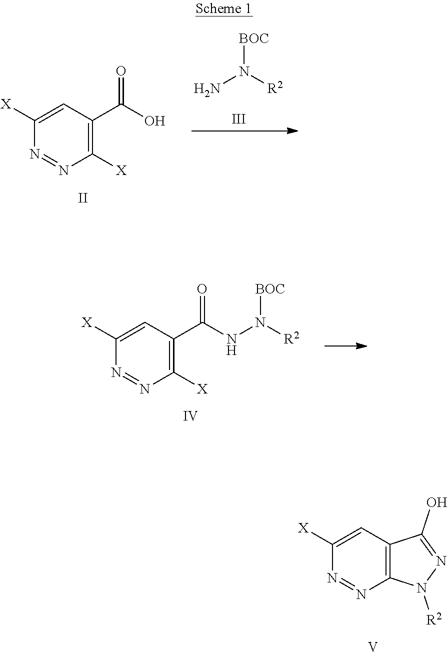

wherein each X is independently halogen.

Compound of formula (V) can be prepared according to Scheme 1. A condensation of compound of formula (II) with compound of formula (III) can generate compound of formula (IV). Treating compound of formula (IV) with an acid (e.g. trifluoroacetic acid, aqueous hydrochloric acid) can provide compound of formula (V).

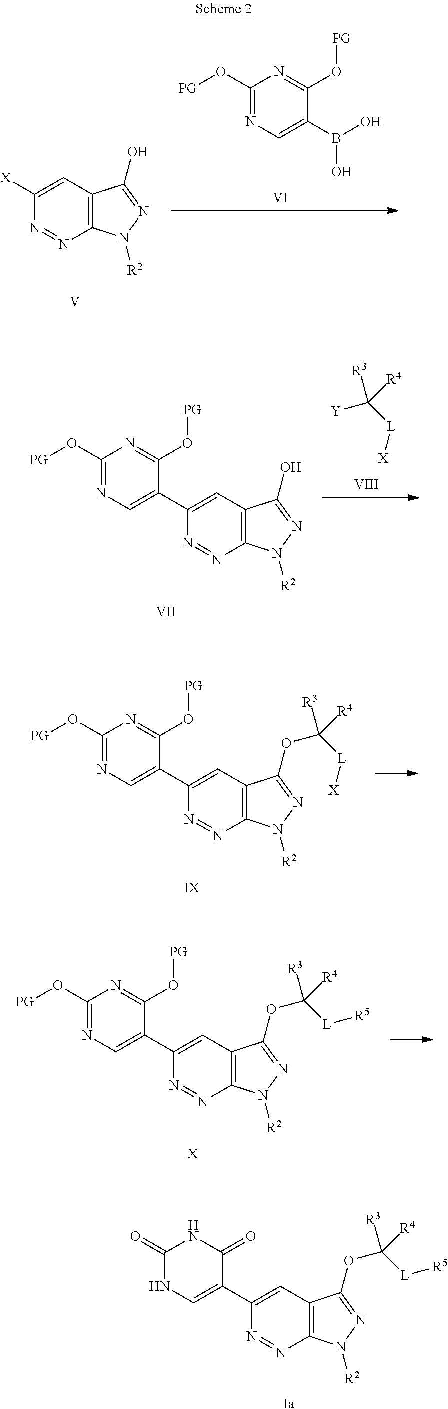

wherein each X is independently halogen; each PG is independently an oxygen protecting group, such as methyl, tert-butyl, TBS, ethoxymethyl and benzyl; Y is OH, OTf, OMs, or halogen.

The compound of formula (VIII) can be obtained according to the synthesis of Intermediate C1 to C3.

Compound of formula (Ia) can be prepared according to Scheme 2. A Suzuki-Miyaura type coupling of compound of formula (V) with heteroaryl boronic acid (VI) in the presence of a palladium catalyst (e.g. Pd(dppf)Cl₂, Pd(PPh₃)₄, cataCXium-A-Pd-G3, etc.) and bases (e.g. Na₂CO₃, K₂CO₃, Cs₂CO₃, etc.) can be conducted to provide compound of formula (VII). The compound of formula (IX) can be obtained via Mitsunobu reaction or nucleophilic substitution of compound of formula (VII) with formula (VIII). The defined R⁵ group of compound of formula (X) can be introduced via transition metal-mediated coupling reaction (e.g. Buchwald-Hartwig, Ullmann-Ma reaction or photo-chemistry). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ia).

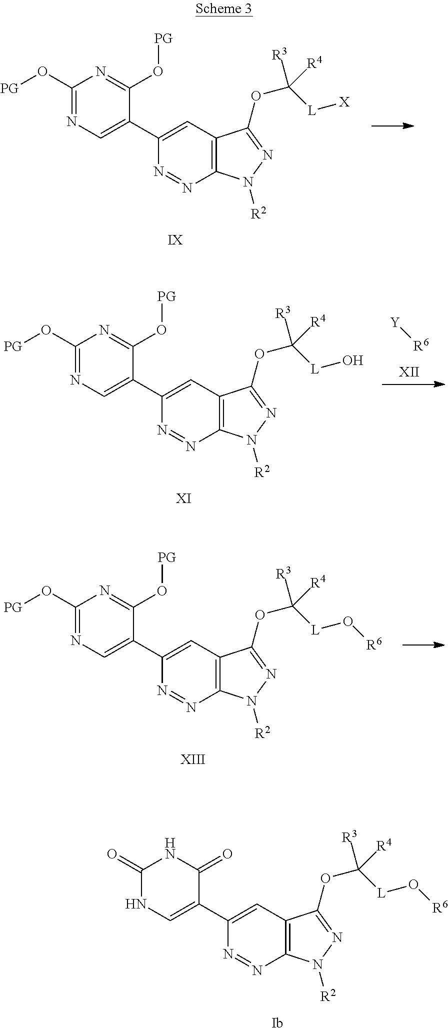

wherein R⁶ is optionally substituted aryl, heteroaryl, heterocyclyl, arylC₁₋₆alkyl, heterocyclylC₁₋₆alkyl or heteroarylC₁₋₆alkyl; X is halogen; Y is OH, OTf, OMs, or halogen.

Compound of formula (Ib) can be prepared according to Scheme 3. The compound of formula (XI) can be obtained via transition metal-mediated coupling reaction of formula (IX) with basic aqueous solution or boronic ester (e.g. B₂pin₂ etc.) followed by oxidation with H₂O₂ (aq), or m-CPBA etc. The compound of formula (XIII) can be obtained via Mitsunobu reaction or nucleophilic substitution of compound of formula (XI) with compound of formula (XII). A following deprotection step using an acid (e.g. trifluoroacetic acid, aqueous hydrochloric acid), or dealkylation reagent (e.g. TMSCl and NaI etc.), or metal (e.g. Pd/C etc.) mediated hydrogenation, can provide the compound of formula (Ib).

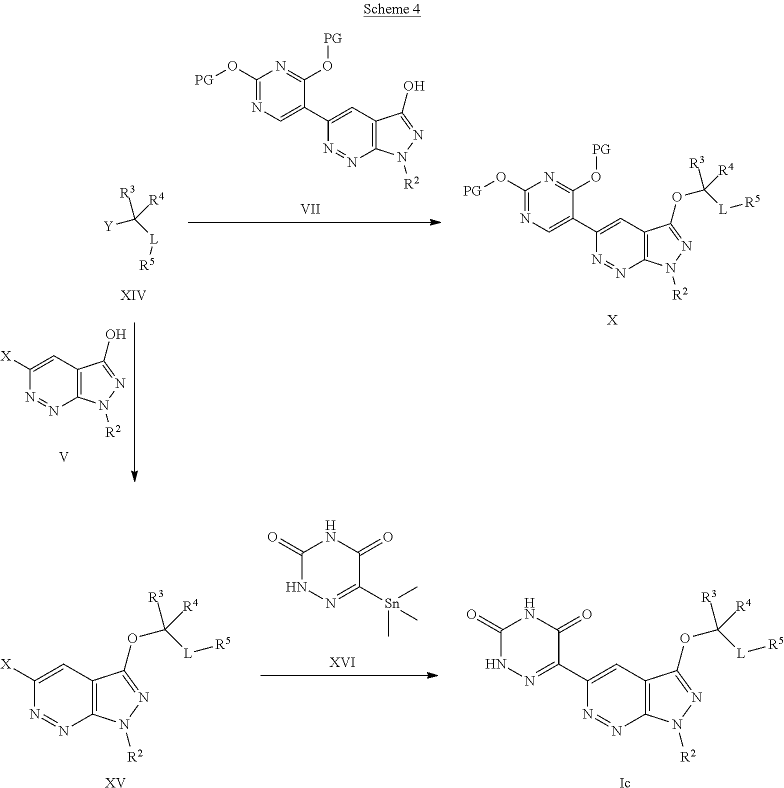

wherein X is halogen; Y is OH, OTf, OMs, or halogen.

Alternatively, the compound of formula (X) can be obtained via Mitsunobu reaction or nucleophilic substitution of compound of formula (VII) with compound of formula (XIV).

Compound of formula (Ic) can be prepared according to Scheme 4. The compound of formula (XV) can be obtained via Mitsunobu reaction or nucleophilic substitution of compound of formula (V) with formula (XIV). A Stille coupling of compound of formula (XV) with organostannane (XVI) in the presence of a palladium catalyst (e.g. Pd(PPh₃)₄, etc.) can be conducted to provide compound of formula (Ic).

Compounds of this invention can be obtained as mixtures of diastereomers or enantiomers, which can be separated by methods well known in the art, e.g. (chiral) HPLC or SFC. In another embodiment, compounds of this invention can be obtained according to above scheme by using corresponding chiral starting materials.

This invention also relates to a process for the preparation of a compound of formula (I) comprising any of the following steps:
a) Deprotection of compound of formula (X),

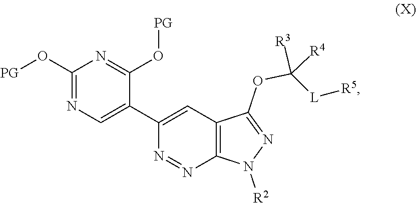

in the presence of an acid, dealkylation reagent, or metal mediated hydrogenation to afford the compound of formula (Ia),

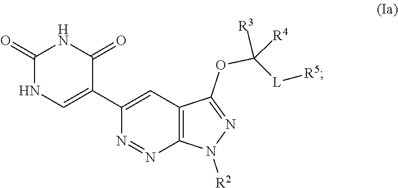

b) Deprotection of compound of formula (XIII),

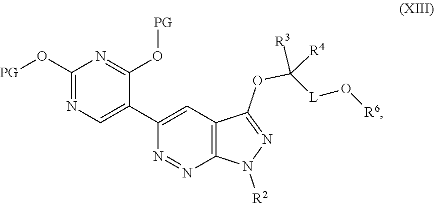

in the presence of an acid, dealkylation reagent, or metal mediated hydrogenation to afford the compound of formula (Ib),

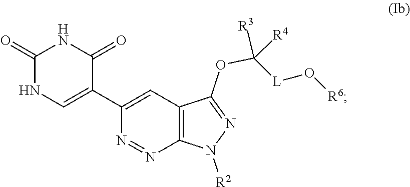

c) Stille coupling of compound of formula (XV),

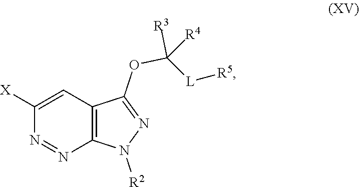

with organostannane (XVI),

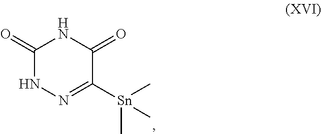

in the presence of a palladium catalyst to afford the compound of formula (Ic),

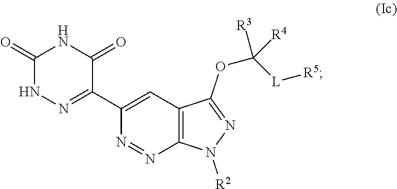

wherein:
X is halogen; each PG is independently an oxygen protecting group; wherein PG can be selected from methyl, tert-butyl, TBS, ethoxymethyl and benzyl; R⁶ is optionally substituted aryl, heteroaryl, heterocyclyl, arylC₁₋₆alkyl, heterocyclylC₁₋₆alkyl or heteroarylC₁₋₆alkyl;
in step a) and b), the acid can be, for example, trifluoroacetic acid or aqueous hydrochloric acid; the dealkylation reagent can be, for example, TMSCl and NaI; the hydrogenation can be conducted with Pd/C;
in step c), the catalyst can be, for example, Pd(PPh₃)₄;

A compound of formula (I) when manufactured according to the above process is also an object of the invention.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
4 Å MS: 4 Å molecular sieve
ACN: acetonitrile
AcOH: acetic acid
DCE: 1,2-dichloroethane
DCM: dichloromethane
DEAD: diethyl azodicarboxylate
DIAD: diisopropyl azodicarboxylate
DIPEA or DIEA: N,N-diisopropylethylamine
DME: dimethoxyethane
DMF: N,N-dimethylformamide
DMP: Dess-Martin periodinane
DtBAD: di-tert-butyl azodicarboxylate
EA or EtOAc: ethyl acetate
FA: formic acid
HATU 1-[bis(dimethylamino)methylene]-1H-1,2,3-triazolo[4,5-b]pyridinium 3-oxide hexafluorophosphate
IC₅₀: half inhibition concentration
Ir[dF(CF₃)ppy]₂(dtbbpy)PF₆: (4,4′-Di-tert-butyl-2,2′-bipyridine)bis[(2-pyridinyl)phenyl]iridium(III) Hexafluorophosphate
LCMS: liquid chromatography-mass spectrometry
m-CPBA: meta-chloroperoxybenzoic acid
MS: mass spectrometry
NBS: N-bromosuccinimide
NiCl₂dtbbpy: [4,4′-bis(1,1-dimethylethyl)-2,2′-bipyridine]nickel dichloride
Pd(dppf)Cl₂·DCM [1,1′-bis(diphenylphosphino)ferrocene]dichloropalladium(II), complex with dichloromethane
PE: petroleum ether
PPh₃: triphenylphosphine
prep-HPLC: preparative high performance liquid chromatography
prep-TLC: preparative thin layer chromatography
rt: room temperature
RT: retention time
RuCl(p-cymene)[(R,R)-Ts-DPEN]: ((R,R)-2-amino-1,2-diphenylethyl)[(4-tolyl)sulfonyl]amido](p-cymene)ruthenium(II) chloride
SFC: supercritical fluid chromatography
Selectfluor N-Chloromethyl-N-fluorotriethylenediammoniumbis (tetrafluoroborate)
STAB: Sodium triacetoxyborohydride
TFA: trifluoroacetic acid
TFE: trifluoroethyl alcohol
t-PentOK: potassium tert-pentoxide
TTMSS: Tris(trimethylsilyl)silane
TLC: thin layer chromatography
TMSCHF₂ (difluoromethyl)trimethylsilane
v/v volume ratio

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), (S,S) whelk-ol, 250×30 mm I.D., TCI Chiral MB-S 250×30 mm I.D., AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A1

### 5-chloro-1-methyl-pyrazolo[3,4-c]pyridazin-3-ol

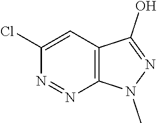

The titled compound was synthesized according to the following scheme:

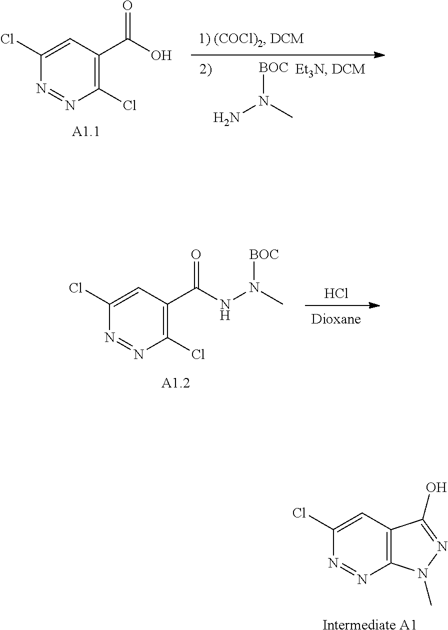

### Step (a): Preparation of tert-butyl N-[(3,6-dichloropyridazine-4-carbonyl)amino]-N-methyl-carbamate (Compound A1.2)

3,6-dichloropyridazine-4-carboxylic acid (compound A1.1, 1.0 g, 5.18 mmol) was suspended in dichloromethane (12.5 mL) and catalytic amount of DMF (5 drops). The mixture was cooled to 0° C. Oxalyl chloride (614 μL, 7.25 mmol) was slowly added and to the mixture was stirred at 0° C. for 20 min. The resultant mixture was warmed to 25° C. and stirred for another 20 min until a clear solution was obtained. The solvent and residual oxalyl chloride were removed entirely under vacuum and the residue was re-dissolved in dichloromethane (12.5 mL). tert-butyl N-amino-N-methyl-carbamate (833.3 mg, 5.7 mmol) and triethylamine (2.22 mL, 15.54 mmol) was dissolved in dichloromethane (12.5 mL) and cooled to 0° C. The above solution of acyl chloride was added dropwise under 0° C., then the reaction mixture was slowly warmed to room temperature, and stirred for 4 h. The reaction was quenched with water (20 mL), extracted with DCM (20 mL) twice. The combined organic layer was dried over Na₂SO₄, filtered and concentrated to afford compound A1.2. MS: calc'd 321.1 [(M+H)⁺], measured 321.1 [(M+H)⁺].

### Step (b) Preparation of 5-chloro-1-methyl-pyrazolo[3,4-c]pyridazin-3-ol (Intermediate A1)

The mixture of above crude compound A1.2 and HCl/dioxane (4 M, 25 mL) was stirred at 55° C. for another 10 h. The resultant precipitation was collected by filtration, the filtrated cake was washed by water (10 mL) for three times, and dried to afford intermediate A1 (762.0 mg). MS: calc'd 185.0 [(M+H)⁺], measured 185.1 [(M+H)⁺].

### Intermediate A2

### 5-chloro-1-(trideuteriomethyl)pyrazolo[3,4-c]pyridazin-3-ol

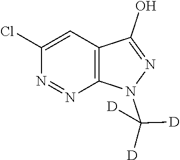 [SMILES: [2H]C([2H])([2H])n1nc(O)c2cc(Cl)nnc21]

The titled compound was synthesized according to the following scheme:

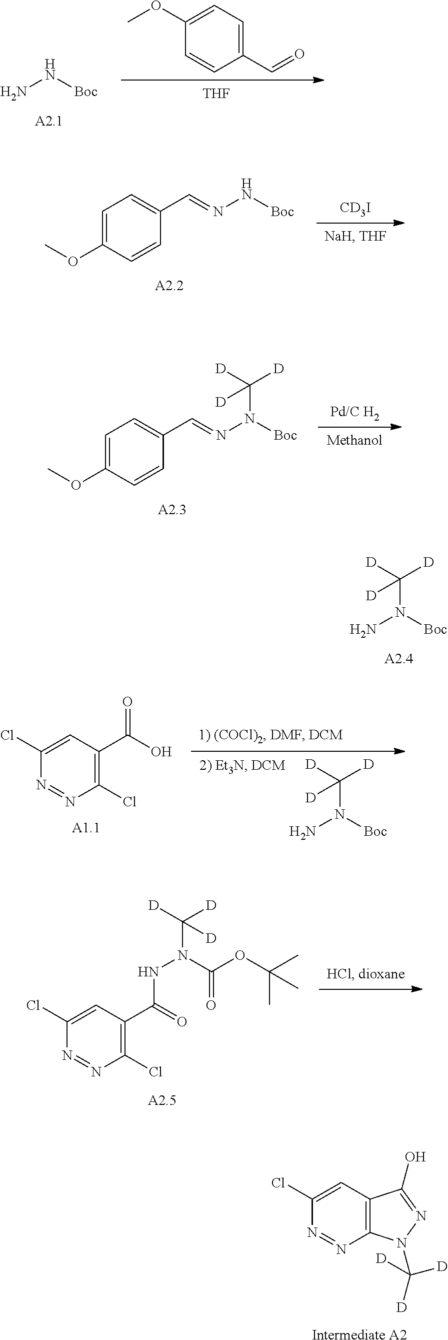

### Step (a): Preparation of tert-butyl N-[(E)-(4-methoxyphenyl)methyleneamino]-carbamate (Compound A2.2)

To a solution of tert-butyl N-aminocarbamate (compound A2.1, 20.0 g, 151 mmol) in THF (100 mL) was added dropwise p-anisaldehyde (20 mL, 166 mmol) at room temperature. The resultant mixture was stirred at room temperature for 16 h. The precipitation was collected by filtration, and dried to afford compound A2.2 (33.0 g). MS: calc'd 251.1 [(M+H)⁺]; measured 251.1 [(M+H)⁺].

### Step (b): Preparation of tert-butyl N-[(E)-(4-methoxyphenyl)methyleneamino]-N-(trideuteriomethyl)carbamate (Compound A2.3)

To a mixture of tert-butyl N-[(E)-(4-methoxyphenyl)methyleneamino]carbamate (compound A2.2, 33.0 g, 131 mmol) and trideuterio(iodo)methane (21.0 g, 145 mmol) in THF (200 ml) was added NaH (60% dispersion in mineral oil, 7.91 g, 198 mmol) at 0° C. under nitrogen atmosphere. The resultant mixture was stirred at room temperature for 2 h, then quenched with ice-water (200 mL), and extracted with EA (200 mL) for three times. The combined organic layer was washed with brine, dried over Na₂SO₄, filtered and concentrated. The residue was purified by silica gel column chromatography (eluted with 0 to 100% EA in PE) to afford compound A2.3 (35.0 g). MS: calc'd 268.2 [(M+H)⁺]; measured 268.3 [(M+H)⁺].

### Step (c): Preparation of tert-butyl N-amino-N-(trideuteriomethyl)carbamate (Compound A2.4)

A mixture of tert-butyl N-[(E)-(4-methoxyphenyl)methyleneamino]-N-(trideuteriomethyl)carbamate (compound A2.3, 35.0 g, 147 mmol) and 10% Pd/C (3.5 g) in methanol (300 mL) was hydrogenated under hydrogen atmosphere at 80° C. for 16 h. After catalyst was filtered off, the filtrate was concentrated in vacuo to afford compound A2.4 (14.3 g). ¹H NMR (400 MHz, Chloroform-d) δ 4.02 (s, 2H), 1.33 (s, 9H).

### Step (d): Preparation of tert-butyl N-[(3,6-dichloropyridazine-4-carbonyl)amino]carbamate (Compound A2.5)

To a solution of 3,6-dichloropyridazine-4-carboxylic acid (compound A1.1, 8.0 g, 41.5 mmol) in DCM (50 mL) was added oxalyl chloride (10.5 g, 82.9 mmol), followed by two drops of DMF. After being stirred at room temperature for 2 h, the reaction mixture was concentrated. And the resultant residue was added to a solution of tert-butyl N-amino-N-(trideuteriomethyl)carbamate (compound A2.4, 7.42 g, 49.7 mmol) and triethylamine (8.3 g, 83.7 mmol) in DCM (50 mL) at 0° C. After being stirred at room temperature for 2 h, the reaction was quenched with water (100 mL), and extracted with DCM (50 mL) for three times. The combined organic layer was washed with brine, dried over Na₂SO₄, filtered and concentrated. The residue was purified by silica gel column chromatography (eluted with 0 to 100% EA in PE) to afford compound A2.5 (11.0 g). MS: calc'd 324.1 [(M+H)⁺]; measured 324.1 [(M+H)⁺].

### Step (e): Preparation of 5-chloro-1-(trideuteriomethyl)pyrazolo[3,4-c]pyridazin-3-ol (Intermediate A2)

The mixture of tert-butyl N-[(3,6-dichloropyridazine-4-carbonyl)amino]carbamate (compound A2.5, 11.0 g, 33.9 mmol) and HCl (4 M in 1,4-dioxane, 60 mL) was stirred at 60° C. for 16 h. The solid was collected by filtration, which was washed by water (50 mL) for three times, and dried to afford intermediate A2 (6.2 g). MS: calc'd 188.0 [(M+H)⁺]; measured 188.0 [(M+H)⁺].

### Intermediate B1

### 5-(2,4-ditert-butoxypyrimidin-5-yl)-1-methyl-pyrazolo[3,4-c]pyridazin-3-ol

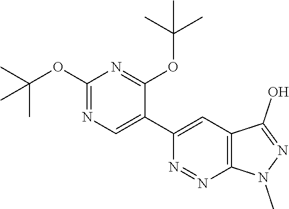 [SMILES: Cn1nc(O)c2cc(-c3cnc(OC(C)(C)C)nc3OC(C)(C)C)nnc21]

The titled compound was synthesized according to the following scheme:

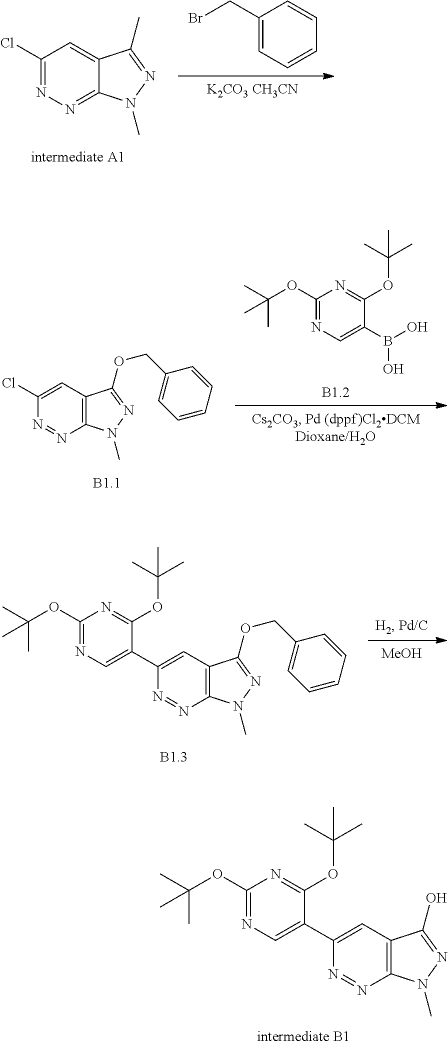

### Step (a): Preparation of 3-benzyloxy-5-chloro-1-methyl-pyrazolo[3,4-c]pyridazine (Compound B1.1)

To a solution of 5-chloro-1-methyl-pyrazolo[3,4-c]pyridazin-3-ol (intermediate A1, 5.0 g, 27.1 mmol) in CH₃CN (100 mL) was added K₂CO₃ (7.5 g, 54.2 mmol) and benzyl bromide (3.2 mL, 27.1 mmol) at room temperature. The resultant mixture was stirred at room temperature for 12 h. The reaction was quenched by slow addition of H₂O (100 mL), then extracted with EA (100 mL) for three times. The combined organic layer was washed with brine (100 mL), dried over anhydrous sodium sulfate, filtered and concentrated in vacuo. The residue was purified by silica gel column chromatography (eluted with PE/EA from 100/1 to 10/1) to afford compound B1.1 (4.2 g). MS: calc'd 275.1, 277.1 [(M+H)⁺], measured 275.2, 277.2 [(M+H)⁺]. ¹H NMR (400 MHz, DMSO-d₆) δ=8.41 (s, 1H), 7.60-7.29 (m, 5H), 5.47 (s, 2H), 4.08 (s, 3H).

### Step (b): Preparation of 3-(benzyloxy)-5-(2,4-di-tert-butoxypyrimidin-5-yl)-1-methyl-1H-pyrazolo[3,4-c]pyridazine (Compound B1.3)

To a solution of 3-benzyloxy-5-chloro-1-methyl-pyrazolo[3,4-c]pyridazine (compound B1.1, 4.20 g, 15.29 mmol) in 1,4-dioxane (100 mL) and H₂O (10 mL) was added (2,4-ditert-butoxypyrimidin-5-yl)boronic acid (compound B1.2, 4.10 g, 15.29 mmol), Cs₂CO₃ (9.96 g, 30.58 mmol) and Pd(dppf)Cl₂·DCM (0.56 g, 0.76 mmol). The resultant mixture was purged with nitrogen for three times, and then stirred at 80° C. for 3 h. After being cooled to room temperature, the reaction mixture was diluted with H₂O (100 mL), and extracted with EA (100 mL) for three times. The combined organic layer was washed with brine (100 mL), dried over anhydrous sodium sulfate, filtered and concentrated. The residue was purified by silica gel column chromatography (eluted with PE/EA from 100/1 to 10/1) to afford compound B1.3 (6.0 g). MS: calc'd 463.2 [(M+H)⁺]; measured 463.2 [(M+H)⁺]. ¹H NMR (400 MHz, DMSO-d₆) δ=8.77 (s, 1H), 8.36 (s, 1H), 7.57-7.48 (m, 2H), 7.46-7.32 (m, 3H), 5.50 (s, 2H), 4.10 (s, 3H), 1.62 (s, 9H), 1.60 (s, 9H).

### Step (c): Preparation of 5-(2,4-di-tert-butoxypyrimidin-5-yl)-1-methyl-1H-pyrazolo[3,4-c]pyridazin-3-ol (Intermediate B1)

A mixture of 3-benzyloxy-5-(2,4-ditert-butoxypyrimidin-5-yl)-1-methyl-pyrazolo[3,4-c]pyridazine (compound B1.3, 6.0 g, 12.97 mmol) and Pd/C (690.23 mg, 10% purity) in methanol (110 mL) was hydrogenated by a hydrogen balloon at room temperature for 2 h. After filtered off the catalyst, the filtrate was concentrated in vacuo to afford intermediate B1 (4.5 g). MS: calc'd 373.2 [(M+H)⁺]; measured 373.3 [(M+H)⁺]. ¹H NMR (400 MHz, DMSO-d₆) δ=11.70 (s, 1H), 8.76 (s, 1H), 8.34 (s, 1H), 4.02 (s, 3H), 1.63 (s, 9H), 1.62 (s, 9H).

### Intermediate B2

### 5-(2,4-ditert-butoxypyrimidin-5-yl)-1-(trideuteriomethyl)pyrazolo[3,4-c]pyridazin-3-ol

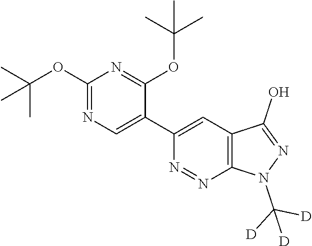 [SMILES: [2H]C([2H])([2H])n1nc(O)c2cc(-c3cnc(OC(C)(C)C)nc3OC(C)(C)C)nnc21]

Intermediate B2 was prepared in analogy to Intermediate B1, by replacing 5-chloro-1-methyl-pyrazolo[3,4-c]pyridazin-3-ol (intermediate A1) with 5-chloro-1-(trideuteriomethyl)pyrazolo[3,4-c]pyridazin-3-ol (intermediate A2) in step (a). MS: calc'd 376.2 [(M+H)⁺]; measured 376.2 [(M+H)⁺].

### Intermediate C1

### (1S)-1-(3-bromo-4-fluoro-phenyl)-2,2-difluoro-ethanol

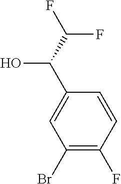 [SMILES: O[C@@H](c1ccc(F)c(Br)c1)C(F)F]

The titled compound was synthesized according to the following scheme:

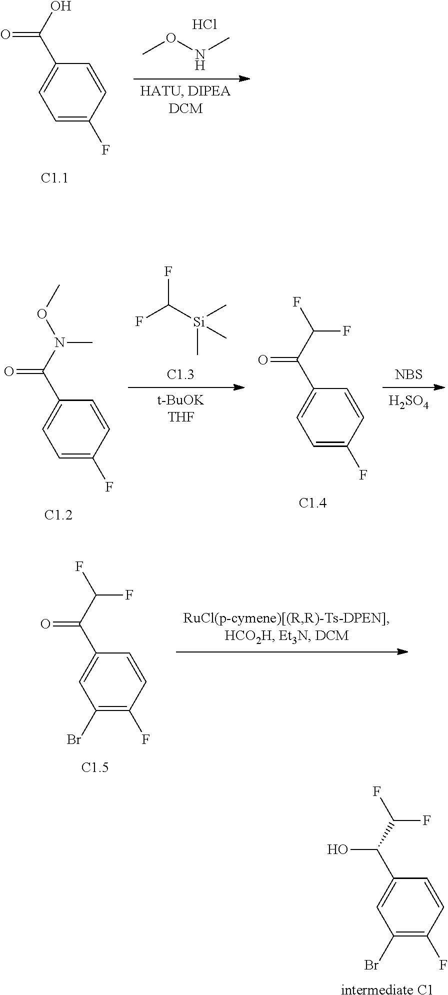

### Step (a): Preparation of 4-fluoro-N-methoxy-N-methyl-benzamide (Compound C1.2)

To a solution of 4-fluorobenzoic acid (compound C1.1, 40.0 g, 285.49 mmol) and DIEA (74.28 g, 570.98 mmol) in DCM (600 mL) was added HATU(119.41 g, 314.04 mmol). The resultant mixture was stirred for 15 min. Then the O,N-dimethylhydroxylamine HCl (30.63 g, 314.04 mmol) was added dropwise into the above mixture, which was stirred at room temperature for another 12 h. The reaction was quenched by slow addition of H₂O (500 mL), and then extracted with DCM (200 mL) for three times. The combined organic layer was washed with brine (100 mL) for three times, dried over anhydrous sodium sulfate, filtered and concentrated. The residue was purified by silica gel column chromatography (eluted with PE/EA from 100/1 to 10/1) to afford compound C1.2 (50.0 g). calc'd 184.1 [(M+H)⁺], measured 184.2 [(M+H)⁺]. ¹H NMR (400 MHz, DMSO-d₆) δ=7.69 (dd, J=5.6, 8.8 Hz, 2H), 7.28 (t, J=8.8 Hz, 2H), 3.54 (s, 3H), 2.69 (s, 3H).

### Step (b): Preparation of 2,2-difluoro-1-(4-fluorophenyl)ethanone (Compound C1.4)

A 1000 mL oven-dried three-necked round-bottom flask equipped with a magnetic stir bar was sealed with a Teflon-lined septum, evacuated, and flame dried under vacuum. The flask was allowed to cool to 25° C., after which it was backfilled with nitrogen. 4-fluoro-N-methoxy-N-methyl-benzamide (compound C1.2, 40.0 g, 218.36 mmol), difluoromethyltrimethylsilane (compound C1.3, 35.26 g, 283.87 mmol) and THF (200 mL) were quickly added. The flask was then evacuated and backfilled with nitrogen for three times. The resultant mixture was cooled to −78° C. t-BuOK (1 M in THF, 393.1 mL, 393.1 mmol) was added dropwise in 1 h at −78° C., and then the reaction mixture was stirred at the same temperature for another 4 h. The reaction was quenched by slow addition of saturated aqueous ammonium chloride (500 mL), and extracted with EA (300 mL) for three times. The combined organic layer was dried over Na₂SO₄, filtered and concentrated. The residue was purified by silica gel chromatography (eluted with PE) to afford compound C1.4 (12.0 g). ¹H NMR (400 MHz, DMSO-d₆) δ=8.19-8.02 (m, 2H), 7.53-7.36 (m, 2H), 7.16 (td, J=52.4, 2.6 Hz, 1H).

### Step (c): Preparation of 1-(3-bromo-4-fluoro-phenyl)-2,2-difluoro-ethanone (Compound C1.5)

To a solution of 2,2-difluoro-1-(4-fluorophenyl)ethanone (compound C1.4, 6.0 g, 34.46 mmol) in H₂SO₄ (60.0 mL) was added NBS (6.75 g, 37.9 mmol). The resultant reaction mixture was stirred at 60° C. for 16 h. After being cooled to room temperature, the reaction mixture was diluted with cold water (300.0 mL), and extracted with EA (150 mL) twice. The combined organic layer was dried over Na₂SO₄, filtered and concentrated. The residue was purified by silica gel chromatography (eluent with PE) to afford compound C1.5 (8.0 g). ¹H NMR (400 MHz, DMSO-d₆) δ 8.35 (dd, J=6.6, 2.1 Hz, 1H), 8.15-8.03 (m, 1H), 7.70-7.55 (m, 1H), 7.19 (td, J=52.4, 2.7 Hz, 1H).

### Step (d): Preparation of (1S)-1-(3-bromo-4-fluoro-phenyl)-2,2-difluoro-ethanol (Intermediate C1)

To a solution of 1-(3-bromo-4-fluoro-phenyl)-2,2-difluoro-ethanone (compound C1.5, 4.0 g, 15.81 mmol) in DCM (40 mL) was added triethylamine (4.41 mL, 31.62 mmol) and formic acid (1.79 mL, 47.43 mmol) at 0° C., followed by addition of a solution of RuCl(p-cymene)[(R,R)-Ts-DPEN] in DCM (2.5 mL). After being stirred at 20° C. for 16 h, the reaction mixture was concentrated, and the resultant residue was purified by silica gel chromatography (eluted with PE/EA from 10/1 to 5/1) to afford intermediate C1 (4.23 g). ¹H NMR (400 MHz, DMSO-d₆) δ 7.75 (d, J=6.8 Hz, 1H), 7.48 (t, J=6.8 Hz, 1H), 7.40 (t, J=8.7 Hz, 1H), 6.36 (d, J=5.2 Hz, 1H), 6.05 (td, J=55.5, 3.7 Hz, 1H), 4.84 (m, 1H).

### Intermediate C2

### (1S)-1-(4-bromo-2-pyridyl)-2,2-difluoro-ethanol

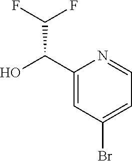 [SMILES: O[C@@H](c1cc(Br)ccn1)C(F)F]

The titled compound was synthesized according to the following scheme:

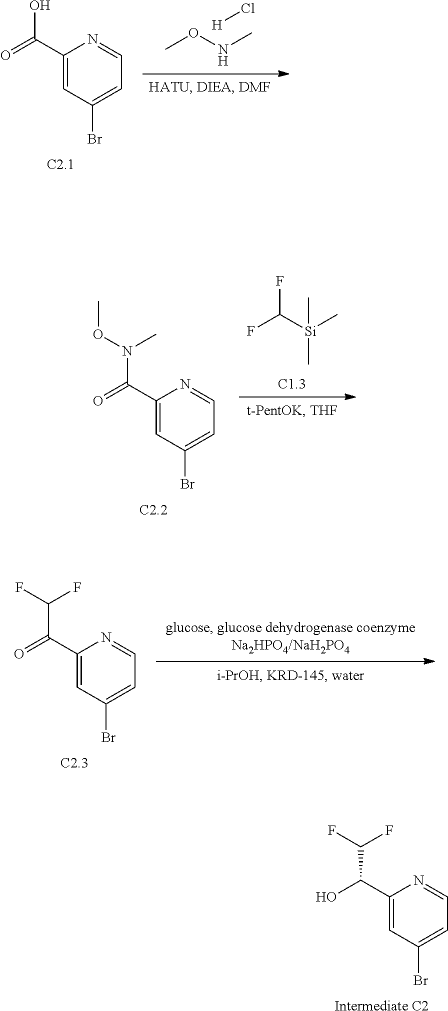

### Step (a): Preparation of 4-bromo-N-methoxy-N-methylpicolinamide (compound C2.2)

A mixture of 4-bromopyridine-2-carboxylic acid (compound C2.1, 25.0 g, 123.8 mmol), DIEA (80 g, 618.8 mmol), N,O-dimethylhydroxylamine hydrochloride (14.5 g, 148.5 mmol) and HATU (94.1 g, 247.5 mmol) in DMF (250 mL) was stirred at room temperature for 12 h. The resultant mixture was diluted with EA (2500 mL). The organic layer was washed with brine (300 mL) for five times, dried over anhydrous Na₂SO₄, filtered and the filtrate was concentrated. The residue was purified by silica gel column chromatography (eluted with PE/EA 1/1) to afford compound C2.2 (24.3 g). calc'd 245.0, 247.0 [(M+H)⁺], measured 245.0, 247.0 [(M+H)⁺].

### Step (b): Preparation of 1-(4-bromopyridin-2-yl)-2,2-difluoroethan-1-one (Compound C2.3)

A mixture of 4-bromo-N-methoxy-N-methyl-pyridine-2-carboxamide (compound C2.2, 10 g, 41.0 mmol) and TMSCF₂H (compound C1.3, 15.2 g, 122 mmol) in THF (100 mL) was stirred for 10 min at −30° C. under nitrogen atmosphere, followed by the addition of t-PentOK (1.0 M in cyclohexane, 82 mL, 82.0 mmol) dropwise at −30° C. The resultant mixture was stirred at −30° C. for 4 h under nitrogen atmosphere. The reaction mixture was diluted with EA (300 mL), washed with saturated NH₄Cl aqueous solution (100 mL) twice, dried over anhydrous Na₂SO₄, filtered and the filtrate was concentrated to afford compound C2.3 (10 g, crude). calc'd 235.9, 237.9 [(M+H)⁺], measured 254.0, 256.0 [(M+H+H₂O)⁺]. ¹H NMR (400 MHz, DMSO-d₆) δ=8.70 (d, J=5.2 Hz, 1H), 8.27 (d, J=1.9 Hz, 1H), 8.08-7.00 (m, 1H), 7.25 (t, J=53.6 Hz, 1H).

### Step (c): Preparation of (1S)-1-(4-bromo-2-pyridyl)-2,2-difluoro-ethanol (Intermediate C2)

A mixture of 1-(4-bromo-2-pyridyl)-2,2-difluoro-ethanone (compound C2.3, 8.0 g, 33.9 mmol), enzyme KRD-145 (Pharmaron, Catalog: KRD-145, 800 mg), glucose (20.8 g), glucose dehydrogenase (2.56 g), coenzyme 11 (4.8 g), Na₂HPO₄ (7.00 g), NaH₂PO₄ (3.80 g) and i-PrOH (48 mL) in water (800 mL) was stirred at 35° C. for 12 h. The mixture was extracted with ethyl acetate (1000 mL) for three times. The combined organic layer was washed with brine (500 mL), dried over anhydrous sodium sulfate, filtered and concentrated. The residue was purified by silica gel column chromatography (eluted with PE/EA 2/1) to afford intermediate C2 (3.6 g). calc'd 238.0, 240.0 [(M+H)⁺], measured 238.0, 240.0 [(M+H)⁺]. ¹H NMR (400 MHz, DMSO-d₆) δ=8.46 (d, J=5.3 Hz, 1H), 7.82-7.63 (m, 2H), 5.91-5.82, (m, 1H) 6.27 (td, J=46.4 Hz, 3.2 Hz, 1H), 4.89-4.82 (m, 3.6 Hz, 1H).

### Intermediate C3

### (1S)-1-(3-bromophenyl)-2,2-difluoro-ethanol

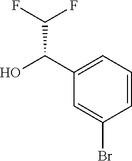 [SMILES: O[C@@H](c1cccc(Br)c1)C(F)F]

The titled compound was synthesized according to the following scheme:

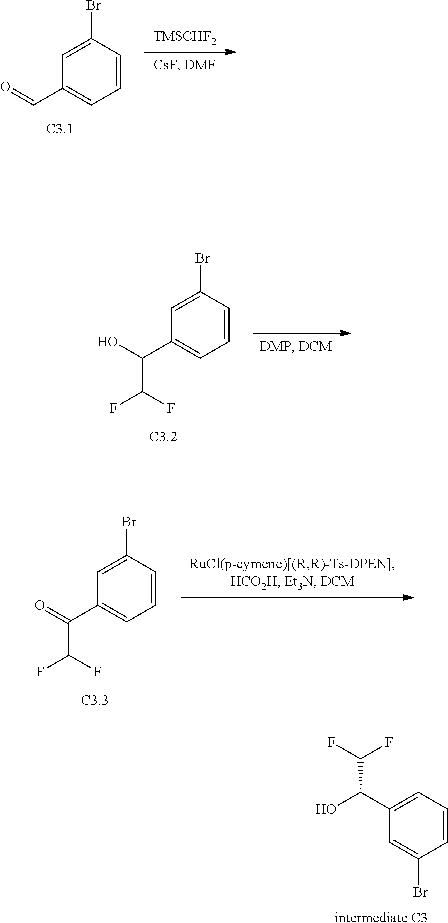

### Step (a): Preparation of 1-(3-bromophenyl)-2,2-difluoro-ethanol (Compound C3.2)

To a solution of 3-bromobenzaldehyde (compound C3.1, 7.9 g, 5.0 mL, 42.7 mmol) in DMF (40 mL) was added TMSCHF₂ (4.0 g, 4.0 mL, 24.5 mmol) and CsF (648.6 mg, 4.3 mmol). The resultant mixture was stirred at room temperature for 2 h under nitrogen. The reaction mixture was diluted with saturated aqueous NaCl (100 mL), and extracted with EA (100 mL) for three times. The combined organic layer was washed with brine, dried over Na₂SO₄, filtered and concentrated in vacuo. The residue was dissolved in THF (20 mL). To the resultant solution was added HF·3Et₃N (4.0 g, 24.5 mmol) at 0° C. After being stirred at 0° C. for 1 h, the reaction was quenched by saturated aqueous NaHCO₃ (100 mL), and extracted with EA (100 mL) for three times. The combined organic layer was washed with brine, dried over Na₂SO₄, filtered and concentrated in vacuo. The residue was purified by flash chromatography (silica gel, 120 g, 0% to 15% EA in PE) to afford compound C3.2 (7.4 g). ¹H NMR (400 MHz, CDCl₃) δ=7.63 (s, 1H), 7.54 (d, J=7.9 Hz, 1H), 7.38 (d, J=7.6 Hz, 1H), 7.31 (d, J=7.8 Hz, 1H), 5.76 (td, J=55.7, 4.6 Hz, 1H), 4.93-4.75 (m, 1H), 2.47 (brd, J=2.8 Hz, 1H).

### Step (b): Preparation of 1-(3-bromophenyl)-2,2-difluoro-ethanone (compound C3.3)

To a solution of 1-(3-bromophenyl)-2,2-difluoro-ethanol (compound C3.2, 7.4 g, 31.3 mmol) in DCM (100 mL) was added DMP (19.9 g, 46.9 mmol). The resultant mixture was stirred at room temperature for 1 h under nitrogen. The reaction mixture was diluted with saturated aqueous NaHCO₃ (100 mL) and saturated aqueous Na₂S₂O₃ (100 mL). After being stirred at room temperature for 1 h, the mixture was extracted with DCM (200 mL) twice. The combined organic layer was washed with saturated aqueous NaHCO₃ and brine, dried over Na₂SO₄, filtered and concentrated in vacuo. The residue was purified by flash chromatography (silica gel, 80 g, 0% to 15% EA in PE) to afford compound C3.3 (6.8 g). ¹H NMR (400 MHz, CDCl₃) δ=8.23 (s, 1H), 8.04 (d, J=7.9 Hz, 1H), 7.86-7.79 (m, 1H), 7.45 (t, J=7.9 Hz, 1H), 6.27 (t, J=51.2 Hz, 1H).

### Step (c): Preparation of (1S)-1-(3-bromophenyl)-2,2-difluoro-ethanol (intermediate C3)

To a solution of 1-(3-bromophenyl)-2,2-difluoro-ethanone (compound C3.3, 2.0 g, 8.5 mmol) in DCM (10 mL) was added HCO₂H (6.0 g, 5.0 mL, 130.4 mmol), and Et₃N (5.2 g, 7.2 mL, 51.7 mmol) at 0° C., followed by the addition of RuCl(p-cymene)[(R,R)-Ts-DPEN](540.8 mg, 850 μmol) in portions at 0° C. After being stirred at room temperature overnight, the reaction mixture was diluted with saturated aqueous NaHCO₃ (100 mL), and extracted with DCM (100 mL) twice. The combined organic layer was washed with brine, dried over Na₂SO₄, filtered and concentrated in vacuo. The residue was purified by flash chromatography (silica gel, 40 g, 0% to 15% EA in PE) to afford intermediate C3 (1.7 g). ¹H NMR (400 MHz, CDCl₃) δ=7.63 (s, 1H), 7.56-7.51 (m, 1H), 7.38 (d, J=7.5 Hz, 1H), 7.31 (d, J=7.8 Hz, 1H), 5.77 (td, J=55.9, 4.5 Hz, 1H), 4.90-4.76 (m, 1H), 2.47 (d, J=3.8 Hz, 1H).

### Intermediate C4

### (1S)-1-(2-bromo-4-pyridyl)-2,2-difluoro-ethanol

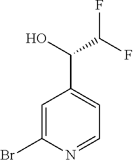 [SMILES: O[C@@H](c1ccnc(Br)c1)C(F)F]

The titled compound was synthesized according to the following scheme:

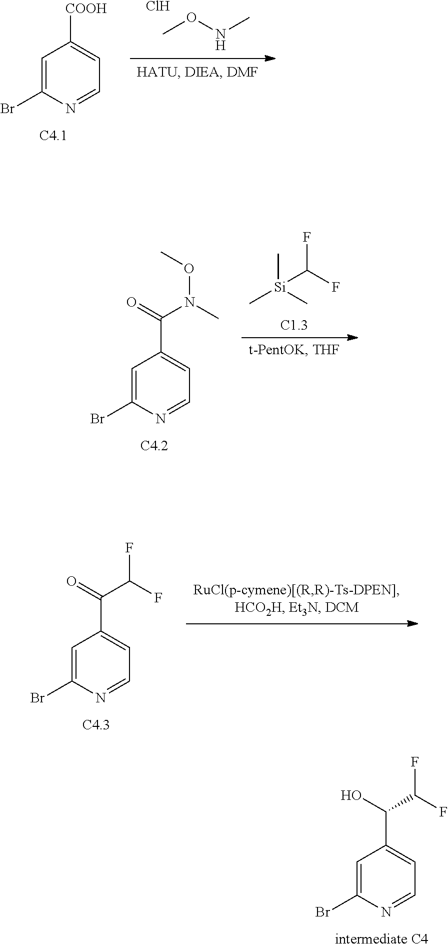

### Step (a): Preparation of 2-bromo-N-methoxy-N-methyl-pyridine-4-carboxamide (Compound C4.2)

To a solution of 2-bromoisonicotinic acid (compound C4.1, 25.0 g, 124 mmol) and DIEA (48.0 g, 371 mmol) in DMF (150 mL) was added HATU (29.1 g, 123 mmol), the resultant mixture was stirred at room temperature for 30 min, and then N,O-dimethylhydroxylamine hydrochloride (12.0 g, 123 mmol) was added. The reaction mixture was stirred at room temperature overnight. The reaction mixture was diluted with water (600 mL), and extracted with EA (600 mL) for three times. Then organic layer was washed with brine (300 mL) for three times, dried over anhydrous Na₂SO₄, filtered and concentrated. The residue was purified by flash chromatography (silica gel, 330 g, 0% to 100% EA in PE) to afford compound C4.2 (19.5 g). MS: calc'd 245.0, 247.0 [(M+H)⁺]; measured 245.0, 247.0 [(M+H)⁺].

### Step (b): Preparation of 1-(2-bromo-4-pyridyl)-2,2-difluoro-ethanone (compound C4.3)

To a solution of 2-bromo-N-methoxy-N-methyl-pyridine-4-carboxamide (compound C4.2, 19.2 g, 78.3 mmol), difluoromethyl)trimethylsilane (9.71 g, 78.3 mmol) in THF (200 mL) was added t-PentOK (2 M in THF, 50.5 ml, 101 mmol) at −60° C. under nitrogen atmosphere. After stirred at −60° C. for 4 h, the reaction mixture was quenched with saturated aqueous ammonium chloride (100 mL), and extracted with EA (300 mL) for three times. Then organic layer was washed with brine (150 mL) for three times, dried over anhydrous Na₂SO₄, filtered and concentrated. The residue was purified by flash chromatography (silica gel, 330 g, 0% to 100% EA in PE) to afford compound C4.3 (12.0 g). MS: calc'd 236.0, 238.0 [(M+H)⁺]; measured 254.0, 256.0 [(M+H+H₂O)⁺].

### Step (c): Preparation of (1S)-1-(2-bromo-4-pyridyl)-2,2-difluoro-ethanol (intermediate C4)

A mixture of 1-(2-bromo-4-pyridyl)-2,2-difluoro-ethanone (compound C4.3, 12.0 g, 50.8 mmol), RuCl[(R,R)-Tsdpen](P-cymene) (321 mg, 0.48 mmol), triethylamine (50.0 g, 0.494 mol) and formic acid (56.5 g, 1.23 mol) was stirred at 40° C. overnight under nitrogen atmosphere. The reaction mixture was diluted with water (400 mL), and extracted with EA (500 mL) for three times. Then organic layer was washed with brine (300 mL) for three times, dried over anhydrous Na₂SO₄, filtered and concentrated. The residue was purified by flash chromatography (silica gel, 120 g, 0% to 100% EA in PE) to afford intermediate C4 (10.8 g). MS: calc'd 238.0, 240.0 [(M+H)⁺]; measured 238.0, 240.0 [(M+H)⁺].

### Intermediate D1

### 3-[(1R)-1-(3-bromo-4-fluoro-phenyl)-2,2-difluoro-ethoxy]-5-(2,4-ditert-butoxypyrimidin-5-yl)-1-methyl-pyrazolo[3,4-c]pyridazine

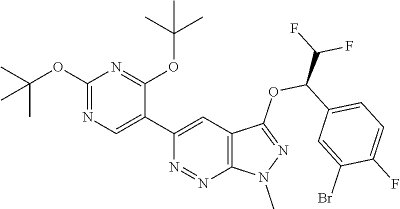 [SMILES: Cn1nc(O[C@H](c2ccc(F)c(Br)c2)C(F)F)c2cc(-c3cnc(OC(C)(C)C)nc3OC(C)(C)C)nnc21]

The titled compound was synthesized according to the following scheme:

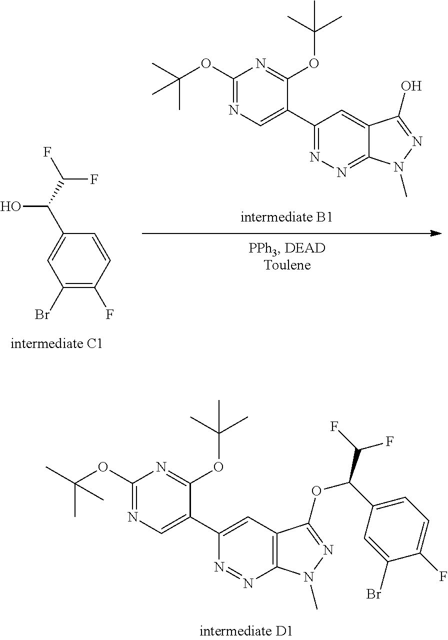

### Step (a): Preparation of 3-[(1R)-1-(3-bromo-4-fluoro-phenyl)-2,2-difluoro-ethoxy]-5-(2,4-ditert-butoxypyrimidin-5-yl)-1-methyl-pyrazolo[3,4-c]pyridazine (intermediate D1)

To a suspension of 5-(2,4-ditert-butoxypyrimidin-5-yl)-1-methyl-pyrazolo[3,4-c]pyridazin-3-ol (intermediate B1, 3.0 g, 8.06 mmol) in toluene (60 mL) was added (1S)-1-(3-bromo-4-fluoro-phenyl)-2,2-difluoro-ethanol (intermediate C1, 2.88 g, 11.28 mmol) and triphenylphosphane (4.23 g, 16.11 mmol). Then diethyl azodicarboxylate (2.93 mL, 16.11 mmol) was added into the mixture dropwise. The reaction mixture was stirred at 60° C. for 2 h. After being cooled to room temperature, the reaction mixture was diluted with water (100 mL), and extracted with EA (100 mL) twice. The combined organic layer was washed with brine, dried over Na₂SO₄, filtered and concentrated. The residue was purified by silica gel chromatography (eluted with PE/EA from 10/1 to 5/1) to afford intermediate D1 (5.0 g). calc'd 609.1, 611.1 [(M+H)⁺], measured 609.4, 611.4 [(M+H)⁺].

### Intermediate D2

### 3-[(1R)-1-(4-bromo-2-pyridyl)-2,2-difluoro-ethoxy]-5-(2,4-ditert-butoxypyrimidin-5-yl)-1-methyl-pyrazolo[3,4-c]pyridazine

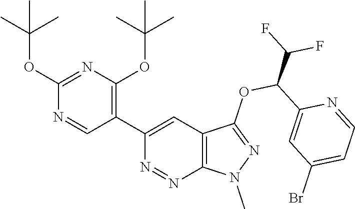 [SMILES: Cn1nc(O[C@H](c2cc(Br)ccn2)C(F)F)c2cc(-c3cnc(OC(C)(C)C)nc3OC(C)(C)C)nnc21]

Intermediate D2 was prepared in analogy to Intermediate D1, by replacing (1S)-1-(3-bromo-4-fluoro-phenyl)-2,2-difluoro-ethanol (intermediate C1) with (1S)-1-(4-bromo-2-pyridyl)-2,2-difluoro-ethanol (intermediate C2) in step (a). calc'd 592.1, 594.1 [(M+H)⁺], measured 592.1, 594.1 [(M+H)⁺].

### Intermediate D3

### 3-[(1R)-1-(3-bromophenyl)-2,2-difluoro-ethoxy]-5-(2,4-ditert-butoxypyrimidin-5-yl)-1-methyl-pyrazolo[3,4-c]pyridazine

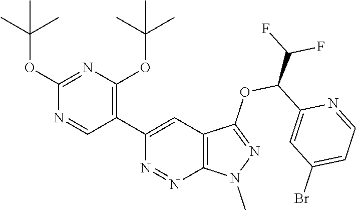 [SMILES: Cn1nc(O[C@H](c2cc(Br)ccn2)C(F)F)c2cc(-c3cnc(OC(C)(C)C)nc3OC(C)(C)C)nnc21]

Intermediate D3 was prepared in analogy to Intermediate D1, by replacing (1S)-1-(3-bromo-4-fluoro-phenyl)-2,2-difluoro-ethanol (intermediate C1) with (1S)-1-(3-bromophenyl)-2,2-difluoro-ethanol (intermediate C3) in step (a). MS: calc'd 591.1 [(M+H)⁺]; measured 477.0 [(M+3H-2tBu)⁺]. ¹H NMR (400 MHz, CDCl₃) δ=9.09 (s, 1H), 8.35 (s, 1H), 7.71 (s, 1H), 7.56 (brd, J=8.1 Hz, 1H), 7.49 (d, J=7.6 Hz, 1H), 7.36-7.30 (m, 1H), 6.35-5.96 (m, 2H), 4.16 (s, 3H), 1.76 (s, 9H), 1.74 (s, 9H).

### Intermediate D4

### 3-[(1R)-1-(4-bromo-2-pyridyl)-2,2-difluoro-ethoxy]-5-(2,4-ditert-butoxypyrimidin-5-yl)-1-(trideuteriomethyl)pyrazolo[3,4-c]pyridazine

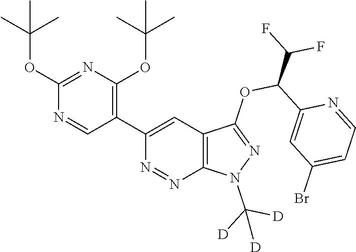 [SMILES: [2H]C([2H])([2H])n1nc(O[C@H](c2cc(Br)ccn2)C(F)F)c2cc(-c3cnc(OC(C)(C)C)nc3OC(C)(C)C)nnc21]

Intermediate D4 was prepared in analogy to Intermediate D1, by replacing (1S)-1-(3-bromo-4-fluoro-phenyl)-2,2-difluoro-ethanol (intermediate C1) and 5-(2,4-ditert-butoxypyrimidin-5-yl)-1-methyl-pyrazolo[3,4-c]pyridazin-3-ol (intermediate B1) with (1S)-1-(4-bromo-2-pyridyl)-2,2-difluoro-ethanol (intermediate C2) and 5-(2,4-ditert-butoxypyrimidin-5-yl)-1-(trideuteriomethyl)pyrazolo[3,4-c]pyridazin-3-ol (intermediate B2) in step (a). MS: calc'd 595.2, 597.2 [(M+H)⁺]; measured 595.2, 597.2 [(M+H)⁺].

### Intermediate D5

### 3-[(1R)-1-(2-bromo-4-pyridyl)-2,2-difluoro-ethoxy]-5-(2,4-ditert-butoxypyrimidin-5-yl)-1-methyl-pyrazolo[3,4-c]pyridazine

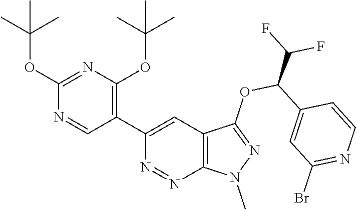 [SMILES: Cn1nc(O[C@H](c2ccnc(Br)c2)C(F)F)c2cc(-c3cnc(OC(C)(C)C)nc3OC(C)(C)C)nnc21]

Intermediate D5 was prepared in analogy to Intermediate D1, by replacing (1S)-1-(3-bromo-4-fluoro-phenyl)-2,2-difluoro-ethanol (intermediate C1) with (1S)-1-(2-bromo-4-pyridyl)-2,2-difluoro-ethanol (intermediate C4) in step (a). MS: calc'd 592.1, 594.1 [(M+H)⁺]; measured 592.2, 594.2 [(M+H)⁺].

### Intermediate E1

### 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol

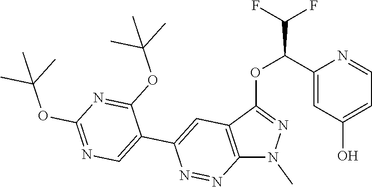 [SMILES: Cn1nc(O[C@H](c2cc(O)ccn2)C(F)F)c2cc(-c3cnc(OC(C)(C)C)nc3OC(C)(C)C)nnc21]

The titled compound was synthesized according to the following scheme:

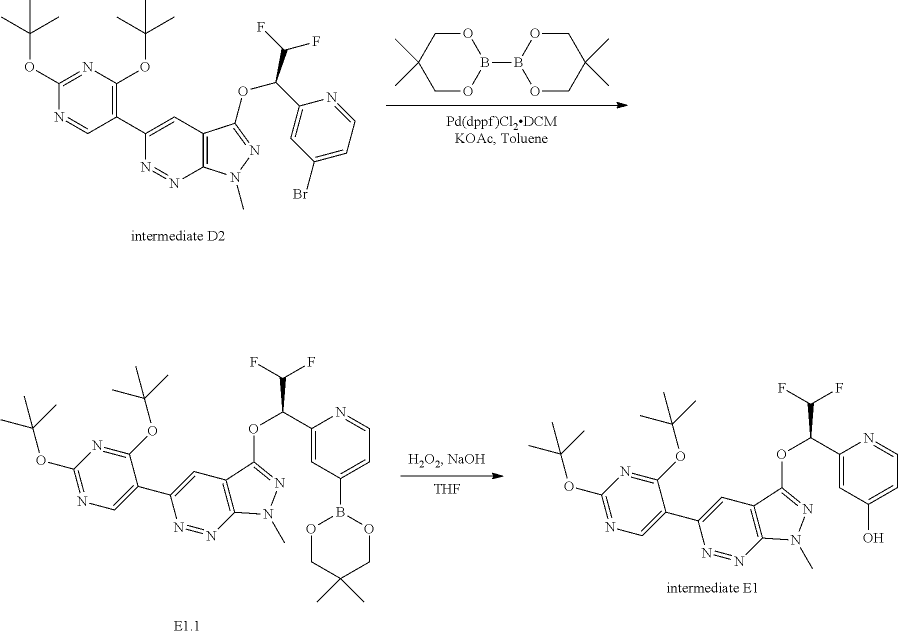

### Step (a): Preparation of 5-(2,4-ditert-butoxypyrimidin-5-yl)-3-[(1R)-1-[4-(5,5-dimethyl-1,3,2-dioxaborinan-2-yl)-2-pyridyl]-2,2-difluoro-ethoxy]-1-methyl-pyrazolo[3,4-c]pyridazine (compound E1.1)

A mixture of 3-[(1R)-1-(4-bromo-2-pyridyl)-2,2-difluoro-ethoxy]-5-(2,4-ditert-butoxypyrimidin-5-yl)-1-methyl-pyrazolo[3,4-c]pyridazine (intermediate D2, 6.0 g, 10.1 mmol), bis (neopentyl glycolato) diboron (11.44 g, 50.6 mmol), potassium acetate (7.0 g, 50.6 mmol) and 1,1′-bis(diphenylphosphino)ferrocene-palladium(II)dichloride dichloromethane complex (826 mg, 1.01 mmol) in toluene (50 mL) was stirred at 100° C. for 16 h under nitrogen. After being cooled to room temperature, the reaction mixture was diluted with water (200 mL), extracted with EA (200 mL) for three times. The combined organic layer was washed with brine, dried over Na₂SO₄, filtered and concentrated. The residue was purified by flash chromatography (eluted with MeOH in DCM from 0 to 10%) to afford compound E1.1(10.98 g). MS: calc'd 626.3 [(M+H)⁺]; measured 626.4 [(M+H)⁺].

### Step (b): Preparation of 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol (intermediate E1)

To a solution of 5-(2,4-ditert-butoxypyrimidin-5-yl)-3-[(1R)-1-[4-(5,5-dimethyl-1,3,2-dioxaborinan-2-yl)-2-pyridyl]-2,2-difluoro-ethoxy]-1-methyl-pyrazolo[3,4-c]pyridazine (compound E1.1, 10.98 g, 17.6 mmol) in THF (183 mL) was added 30% hydrogen peroxide (20.3 g, 18.3 mL) and 3 N NaOH(aq.) (18.3 mL, 54.9 mmol) dropwise at 0° C. After being stirred at room temperature for 16 h, the reaction mixture was adjusted to pH 6 with 1 N HCl (aq.), and then extracted with DCM (300 mL) for three times. The combined organic layer was washed with brine, dried over Na₂SO₄, filtered and concentrated. The residue was purified by silica gel column chromatography (eluted with MeOH/DCM from 0 to 10%) to afford intermediate E1 (4.32 g). MS: calc'd 530.2 [(M+H)⁺]; measured 530.2 [(M+H)⁺].

### Intermediate E2

### 2-[(1R)-1-[5-(2,4-ditert-butoxypyrimidin-5-yl)-1-(trideuteriomethyl)pyrazolo[3,4-c]pyridazin-3-yl]oxy-2,2-difluoro-ethyl]pyridin-4-ol

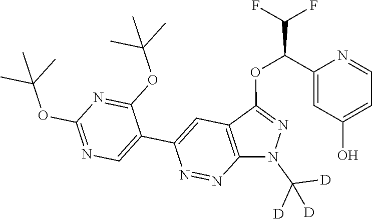 [SMILES: [2H]C([2H])([2H])n1nc(O[C@H](c2cc(O)ccn2)C(F)F)c2cc(-c3cnc(OC(C)(C)C)nc3OC(C)(C)C)nnc21]

Intermediate E2 was prepared in analogy to Intermediate E1, by replacing 3-[(1R)-1-(4-bromo-2-pyridyl)-2,2-difluoro-ethoxy]-5-(2,4-ditert-butoxypyrimidin-5-yl)-1-methyl-pyrazolo[3,4-c]pyridazine (intermediate D2) with 3-[(1R)-1-(4-bromo-2-pyridyl)-2,2-difluoro-ethoxy]-5-(2,4-ditert-butoxypyrimidin-5-yl)-1-(trideuteriomethyl)pyrazolo[3,4-c]pyridazine (intermediate D4) in step (a). MS: calc'd 533.2 [(M+H)⁺]; measured 533.2 [(M+H)⁺].

### Intermediate E3

### 5-[(1R)-1-[5-(2,4-ditert-butoxypyrimidin-5-yl)-1-methyl-pyrazolo[3,4-c]pyridazin-3-yl]oxy-2,2-difluoro-ethyl]-2-fluoro-phenol

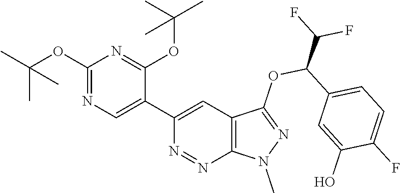 [SMILES: Cn1nc(O[C@H](c2ccc(F)c(O)c2)C(F)F)c2cc(-c3cnc(OC(C)(C)C)nc3OC(C)(C)C)nnc21]

The titled compound was synthesized according to the following scheme:

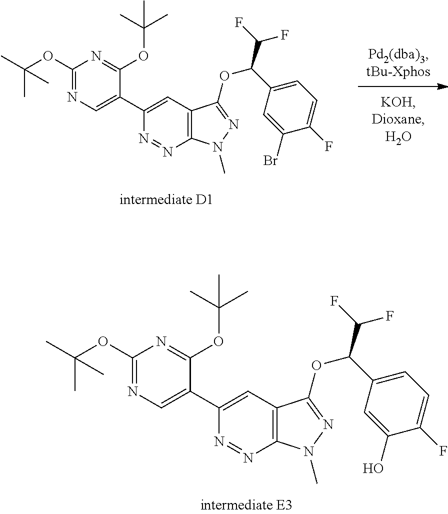 [SMILES: Cn1nc(O[C@H](c2ccc(F)c(Br)c2)C(F)F)c2cc(-c3cnc(OC(C)(C)C)nc3OC(C)(C)C)nnc21 ; Cn1nc(O[C@H](c2ccc(F)c(O)c2)C(F)F)c2cc(-c3cnc(OC(C)(C)C)nc3OC(C)(C)C)nnc21]

### Step (a): Preparation of 5-[(1R)-1-[5-(2,4-ditert-butoxypyrimidin-5-yl)-1-methyl-pyrazolo[3,4-c]pyridazin-3-yl]oxy-2,2-difluoro-ethyl]-2-fluoro-phenol (intermediate E3)

To the mixture of 3-[(1R)-1-(3-bromo-4-fluoro-phenyl)-2,2-difluoro-ethoxy]-5-(2,4-ditert-butoxypyrimidin-5-yl)-1-methyl-pyrazolo[3,4-c]pyridazine (intermediate D1, 100 mg, 164.1 μmol) and potassium hydroxide (92.1 mg, 1.6 mmol) in 1,4-dioxane (1 mL) and water (0.5 mL) were added 2-di-tert-butylphosphino-2′,4′,6′-triisopropylbiphenyl (14.0 mg, 32.8 μmol) and tris(dibenzylideneacetone)dipalladium (30.1 mg, 32.8 μmol). The mixture was stirred at 100° C. under nitrogen for 1.5 h. After being cooled to room temperature, the reaction mixture was acidified with 1N HCl (aq.) to pH about 6, and then extracted with EA (10 mL) for three times. The combined organic layer was dried over Na₂SO₄ and concentrated. The residue was purified by silica gel chromatography (eluted with EA/PE from 0 to 4/1) to afford intermediate E3 (80.0 mg). calc'd 547.2 [(M+H)⁺], measured 547.3 [(M+H)⁺].

### Example 1

### 5-[3-[(1R)-2,2-difluoro-1-[3-(2,2,2-trifluoroethoxy)phenyl]ethoxy]-1-methyl-pyrazolo[3,4-c]pyridazin-5-yl]-1H-pyrimidine-2,4-dione

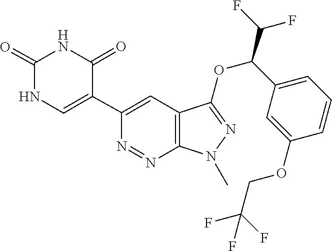 [SMILES: Cn1nc(O[C@H](c2cccc(OCC(F)(F)F)c2)C(F)F)c2cc(-c3c[nH]c(=O)[nH]c3=O)nnc21]

The titled compound was synthesized according to the following scheme:

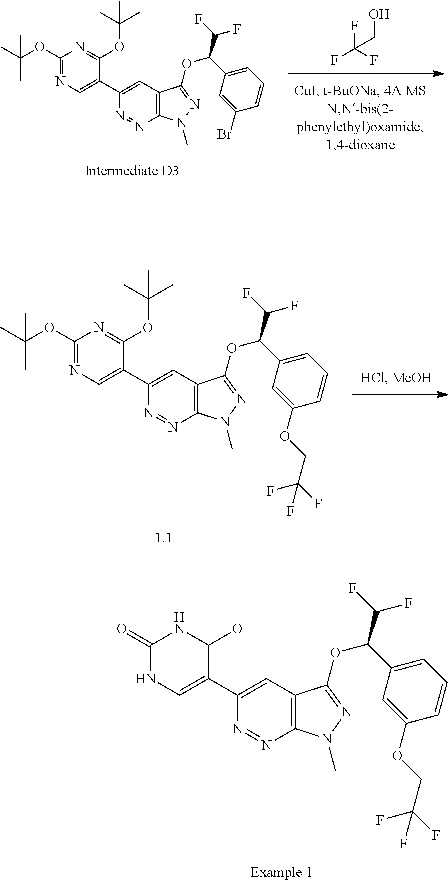 [SMILES: Cn1nc(O[C@H](c2cccc(Br)c2)C(F)F)c2cc(-c3cnc(OC(C)(C)C)nc3OC(C)(C)C)nnc21 ; OCC(F)(F)F ; Cn1nc(O[C@H](c2cccc(OCC(F)(F)F)c2)C(F)F)c2cc(-c3cnc(OC(C)(C)C)nc3OC(C)(C)C)nnc21 ; Cn1nc(O[C@H](c2cccc(OCC(F)(F)F)c2)C(F)F)c2cc(C3=CNC(=O)NC3O)nnc21]

### Step (a): Preparation of 5-(2,4-ditert-butoxypyrimidin-5-yl)-3-[(1R)-2,2-difluoro-1-[3-(2,2,2-trifluoroethoxy)phenyl]ethoxy]-1-methyl-pyrazolo[3,4-c]pyridazine (compound 1.1)

To a solution of 3-[(1R)-1-(3-bromophenyl)-2,2-difluoro-ethoxy]-5-(2,4-ditert-butoxypyrimidin-5-yl)-1-methyl-pyrazolo[3,4-c]pyridazine (intermediate D3) (200.0 mg, 338.2 μmol) in 1,4-dioxane (1 mL) was added 4 Å MS (300 mg), 2,2,2-trifluoroethanol (125.2 mg, 90.0 μL, 1.3 mmol), N,N′-bis(2-phenylethyl)oxamide (11.0 mg, 37.2 μmol), t-BuONa (81.2 mg, 845.4 μmol) and CuI (6.4 mg, 33.8 μmol). The resultant mixture was stirred at 90° C. for 14 h. After being cooled to room temperature, the reaction mixture was diluted with H₂O (10 mL), and extracted with EA (10 mL) for three times. The combined organic layer was washed with brine, dried over Na₂SO₄, filtered and concentrated in vacuo. The residue was purified by flash chromatography (silica gel, 24 g, 0% to 25% EA in PE) to afford compound 1.1 (183.7 mg). MS: calc'd 611.2 [(M+H)⁺]; measured 499.1 [(M+3H-2tBu)⁺].

### Step (b): Preparation of 5-[3-[(1R)-2,2-difluoro-1-[3-(2,2,2-trifluoroethoxy)phenyl]ethoxy]-1-methyl-pyrazolo[3,4-c]pyridazin-5-yl]-1H-pyrimidine-2,4-dione (Example 1)

To the mixture of 5-(2,4-ditert-butoxypyrimidin-5-yl)-3-[(1R)-2,2-difluoro-1-[3-(2,2,2-trifluoroethoxy)phenyl]ethoxy]-1-methyl-pyrazolo[3,4-c]pyridazine (compound 1.1, 183.7 mg, 300.9 μmol) in methanol (1.5 mL) was added HCl (2.0 M in MeOH, 0.4 mL, 0.8 mmol). The mixture was stirred at 22° C. for 2 hr. The reaction mixture was concentrated to afford a crude product, which was purified by pre-HPLC to afford Example 1 (20.4 mg). MS: calc'd 499.1 [(M+H)⁺]; measured 499.1 [(M+H)⁺]. ¹H NMR (400 MHz, DMSO-d₆) δ=11.88-10.91 (m, 2H), 8.63 (s, 1H), 8.29 (s, 1H), 7.33 (t, J=7.9 Hz, 1H), 7.26-7.18 (m, 2H), 7.03 (dd, J=8.2, 2.1 Hz, 1H), 6.52 (td, J=54.0, 4.0 Hz, 1H), 6.11 (td, J=10.6, 3.0 Hz, 1H), 4.70 (q, J=8.8 Hz, 2H), 3.95 (s, 3H).

### Example 2

### 5-[3-[(1R)-1-[2-(cyclopropoxymethyl)-4-pyridyl]-2,2-difluoro-ethoxy]-1-methyl-pyrazolo[3,4-c]pyridazin-5-yl]-1H-pyrimidine-2,4-dione

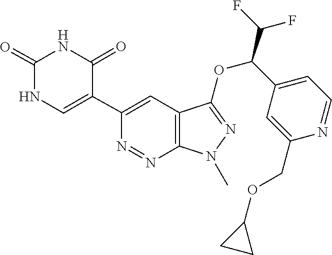 [SMILES: Cn1nc(O[C@H](c2ccnc(COC3CC3)c2)C(F)F)c2cc(-c3c[nH]c(=O)[nH]c3=O)nnc21]

The titled compound was synthesized according to the following scheme:

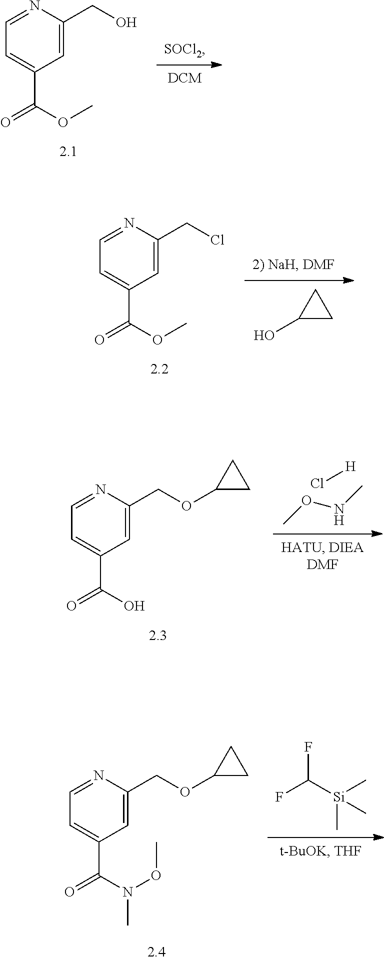

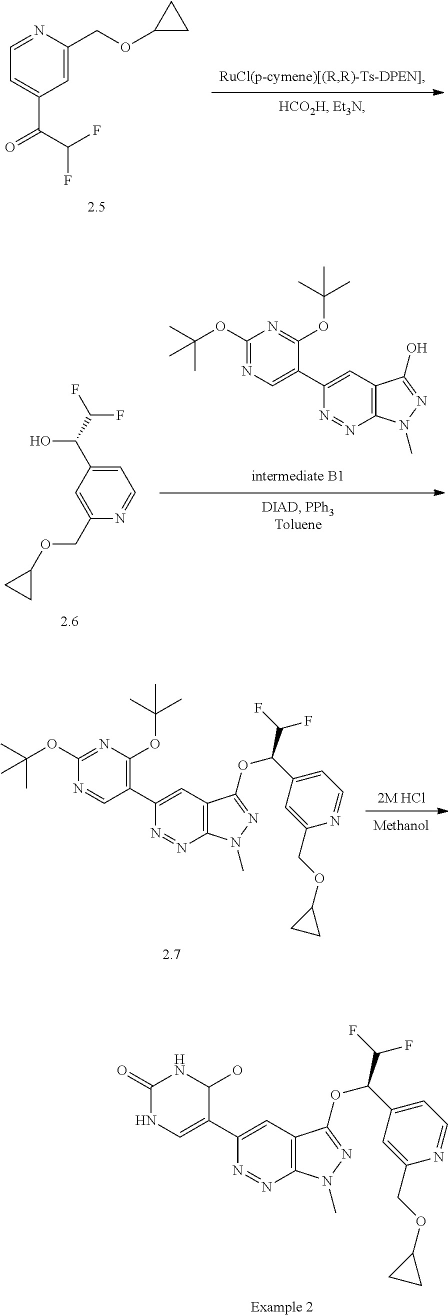

### Step (a): Preparation of methyl 2-(chloromethyl)pyridine-4-carboxylate (compound 2.2)

To a solution of 2-methylolisonicotinic acid methyl ester (compound 2.1, 910 mg, 5.44 mmol) in DCM (20 mL) was added dropwise thionyl chloride (471.02 μL, 6.53 mmol) at 0° C. The resultant mixture was stirred at 0° C. for 0.5 hr. The reaction mixture was concentrated to afford compound 2.2 (1.0 g) which was used for next step directly.

### Step (b): Preparation of 2-(cyclopropoxymethyl)pyridine-4-carboxylic acid (Compound 2.3)

NaH (60% dispersion in mineral oil, 474.11 mg, 11.85 mmol) was added to the solution of cyclopropanol (469.37 mg, 8.08 mmol) in DMF (10 mL) at 0° C. After the reaction mixture was stirred at the same temperature for 0.5 h, 2-(chloromethyl)pyridine-4-carboxylate (compound 2.2, 1000 mg, 5.39 mmol) was added, and the resultant mixture was stirred at room temperature for another 12 h. The reaction was acidified with 2N HCl to pH about 6, and then extracted with EA (30 mL) for three times. The combined organic layer was washed with brine, dried over Na₂SO₄, filtered and concentrated. The residue was purified by flash chromatography (silica gel, 12 g, eluted with 0% to 8% Methanol in DCM) to afford compound 2.3 (360 mg). MS: calc'd 194.1 [(M+H)⁺]; measured 194.1 [(M+H)⁺].

### Step (c): Preparation of 2-(cyclopropoxymethyl)-N-methoxy-N-methyl-pyridine-4-carboxamide (compound 2.4)

To a solution of 2-(cyclopropoxymethyl)pyridine-4-carboxylic acid (compound 2.3, 360 mg, 1.86 mmol) and DIEA (963.28 mg, 1.3 mL, 7.45 mmol) in dichloromethane (8 mL) was added HATU (779.35 mg, 2.05 mmol). After the reaction mixture was stirred at room temperature for 0.5 h, N-methoxymethanamine; hydrochloride (218.1 mg, 2.24 mmol) was added, and the resultant mixture was stirred at 20° C. for 16 hr, then quenched with water (5 mL), and extracted with DCM (10 mL) for three times. The combined organic layer was dried over Na₂SO₄ and concentrated. The residue was purified by flash chromatography (silica gel, 12 g, eluted with 0% to 50% EA in PE) to afford compound 2.4 (300 mg). MS: calc'd 237.1 [(M+H)⁺]; measured 237.1 [(M+H)⁺].

### Step (d): Preparation of 1-[2-(cyclopropoxymethyl)-4-pyridyl]-2,2-difluoro-ethanone (Compound 2.5)

To a solution of 2-(cyclopropoxymethyl)-N-methoxy-N-methyl-pyridine-4-carboxamide (compound 2.4, 250 mg, 1.06 mmol) and difluoromethyltrimethylsilane (262.83 mg, 2.12 mmol) in THF (2.5 mL) was added potassium tert-butoxide (296.83 mg, 2.65 mmol) at −10° C. The resultant mixture was stirred at the same temperature for another 1 hr. The reaction mixture was quenched with saturated aqueous NH₄Cl (2 mL), extracted with EA (10 mL) for three times. The combined organic layer was dried over Na₂SO₄, filtered and concentrated. The residue was purified by flash chromatography (silica gel, eluted with 10% to 70% EA in PE) to afford compound 2.5 (150 mg). MS: calc'd 228.1 [(M+H)⁺]; measured 246.1 [(M+H₂O+H)⁺].

### Step (e): Preparation of (1S)-1-[2-(cyclopropoxymethyl)-4-pyridyl]-2,2-difluoro-ethanol (Compound 2.6)

To a solution of 1-[2-(cyclopropoxymethyl)-4-pyridyl]-2,2-difluoro-ethanone (compound 2.5, 150 mg, 660.18 μmol) in DCM (4 mL) was added formic acid (911.65 mg, 759.7 μL, 19.81 mmol) and triethylamine (801.65 mg, 1.1 mL, 7.92 mmol). Then RuCl(p-cymene)[(R,R)-Ts-DPEN](84.0 mg, 132.04 μmol) was added and the mixture was stirred at 20° C. for 1 hr. The resultant mixture was concentrated, and the residue was purified by flash chromatography (silica gel, eluted with 0% to 40% EA in PE) to afford compound 2.6 (145 mg). MS: calc'd 230.1 [(M+H)⁺]; measured 230.0 [(M+H)⁺].

### Step (f): Preparation of 3-[(1R)-1-[2-(cyclopropoxymethyl)-4-pyridyl]-2,2-difluoro-ethoxy]-5-(2,4-ditert-butoxypyrimidin-5-yl)-1-methyl-pyrazolo[3,4-c]pyridazine (compound 2.7)

To a suspension of (1S)-1-[2-(cyclopropoxymethyl)-4-pyridyl]-2,2-difluoro-ethanol (compound 2.6, 145 mg, 632.58 μmol), 5-(2,4-ditert-butoxypyrimidin-5-yl)-1-methyl-pyrazolo[3,4-c]pyridazin-3-ol (intermediate B1, 190 mg, 510.18 μmol) and triphenylphosphine (160.58 mg, 612.21 μmol) in toluene (3.8 mL) was added dropwise DIAD (106.62 mg, 612.21 μmol). The resultant mixture was stirred at 60° C. for 0.5 h under nitrogen. After being cooled to room temperature, the reaction mixture was diluted with water (50 mL), extracted with EA (30 mL) for three times. The combined organic layer was washed with brine, dried over Na₂SO₄, filtered and concentrated. The residue was purified by flash chromatography (silica gel, 40 g, eluted with 0% to 45% EA in PE) to afford compound 2.7 (60 mg). MS: calc'd 584.3 [(M+H)⁺]; measured 584.5 [(M+H)⁺].

### Step (g): Preparation of 5-[3-[(1R)-1-[2-(cyclopropoxymethyl)-4-pyridyl]-2,2-difluoro-ethoxy]-1-methyl-pyrazolo[3,4-c]pyridazin-5-yl]-1H-pyrimidine-2,4-dione (Example 2)

To the mixture of 3-[(1R)-1-[2-(cyclopropoxymethyl)-4-pyridyl]-2,2-difluoro-ethoxy]-5-(2,4-ditert-butoxypyrimidin-5-yl)-1-methyl-pyrazolo[3,4-c]pyridazine (compound 2.7, 60 mg, 102.8 μmol) in methanol (1 mL) was added 2 M HCl (154.21 μL, 308.42 μmol). The reaction mixture was stirred at room temperature for 1 h, then concentrated to afford a crude product, which was purified by pre-HPLC to afford Example 2 (37.9 mg). MS: calc'd 472.1 [(M+H)⁺]; measured 472.1 [(M+H)⁺]. ¹H NMR (400 MHz, DMSO-d₆) δ=11.55 (d, J=1.6 Hz, 1H), 11.47 (br d, J=6.1 Hz, 1H), 8.73 (s, 1H), 8.61 (d, J=5.3 Hz, 1H), 8.38 (d, J=6.3 Hz, 1H), 7.69 (s, 1H), 7.60 (br d, J=5.0 Hz, 1H), 6.66 (td, J=53.7, 2.5 Hz, 1H), 6.34 (td, J=11.9, 2.1 Hz, 1H), 4.64 (s, 2H), 4.01 (s, 3H), 3.46-3.38 (m, 1H), 0.58-0.41 (m, 4H).

### Example 3

### 5-[3-[(1R)-2,2-difluoro-1-[4-fluoro-3-(2,2,2-trifluoroethoxy)phenyl]ethoxy]-1-methyl-pyrazolo[3,4-c]pyridazin-5-yl]-1H-pyrimidine-2,4-dione

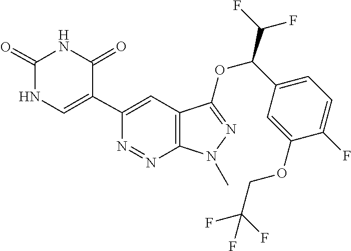 [SMILES: Cn1nc(O[C@H](c2ccc(F)c(OCC(F)(F)F)c2)C(F)F)c2cc(-c3c[nH]c(=O)[nH]c3=O)nnc21]

The titled compound was synthesized according to the following scheme:

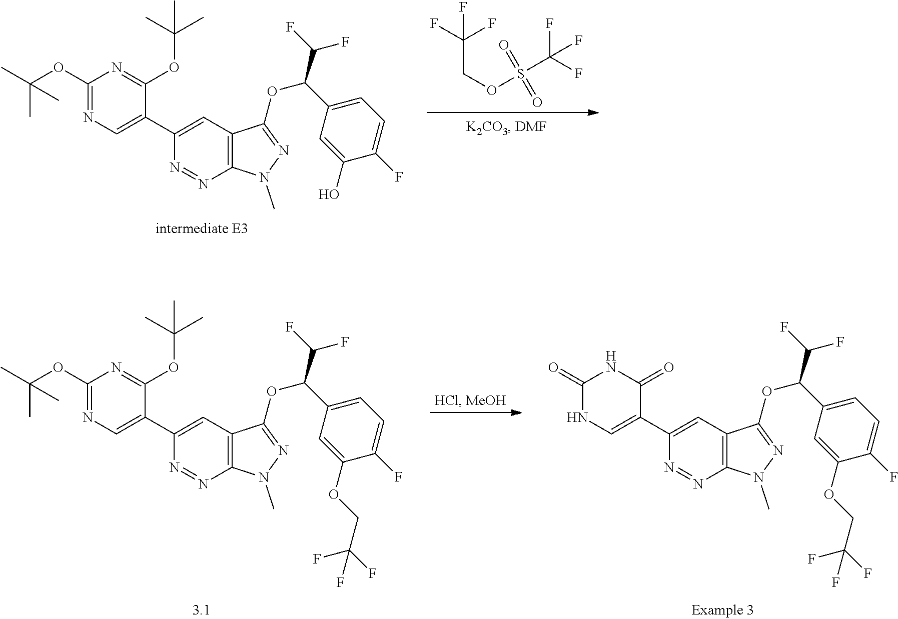 [SMILES: Cn1nc(O[C@H](c2ccc(F)c(O)c2)C(F)F)c2cc(-c3cnc(OC(C)(C)C)nc3OC(C)(C)C)nnc21 ; O=S(=O)(OCC(F)(F)F)C(F)(F)F ; Cn1nc(O[C@H](c2ccc(F)c(OCC(F)(F)F)c2)C(F)F)c2cc(-c3cnc(OC(C)(C)C)nc3OC(C)(C)C)nnc21 ; Cn1nc(O[C@H](c2ccc(F)c(OCC(F)(F)F)c2)C(F)F)c2cc(-c3c[nH]c(=O)[nH]c3=O)nnc21]

### Step (a): Preparation of 5-(2,4-ditert-butoxypyrimidin-5-yl)-3-[(1R)-2,2-difluoro-1-[4-fluoro-3-(2,2,2-trifluoroethoxy)phenyl]ethoxy]-1-methyl-pyrazolo[3,4-c]pyridazine (Compound 3.1)

To the mixture of 5-[(1R)-1-[5-(2,4-ditert-butoxypyrimidin-5-yl)-1-methyl-pyrazolo[3,4-c]pyridazin-3-yl]oxy-2,2-difluoro-ethyl]-2-fluoro-phenol (intermediate E3, 80 mg, 146.4 μmol) and 2,2,2-trifluoroethyl trifluoromethanesulfonate (40.8 mg, 175.7 μmol) in DMF (2 mL) was added potassium carbonate (40.5 mg, 292.8 μmol). The mixture was stirred at room temperature for 16 h. The reaction was quenched with H₂O (3 mL), and was extracted with EA (10 mL) for three times. The combined organic layer was washed with H₂O (5 mL) three times, dried over Na₂SO₄ and concentrated. The residue was purified by silica gel chromatography (eluted with PE/EA from 0/1 to 3/1) to afford compound 3.1 (72.0 mg). calc'd 629.2 [(M+H)⁺], measured 629.4 [(M+H)⁺].

### Step (b): Preparation of 5-[3-[(1R)-2,2-difluoro-1-[4-fluoro-3-(2,2,2-trifluoroethoxy)phenyl]ethoxy]-1-methyl-pyrazolo[3,4-c]pyridazin-5-yl]-1H-pyrimidine-2,4-dione (Example 3)

To a solution of 5-(2,4-ditert-butoxypyrimidin-5-yl)-3-[(1R)-2,2-difluoro-1-[4-fluoro-3-(2,2,2-trifluoroethoxy)phenyl]ethoxy]-1-methyl-pyrazolo[3,4-c]pyridazine (compound 3.1, 72 mg, 114.6 μmol) in methanol (0.5 mL) was added 2 N HCl (aq.) (171.8 μL, 343.6 μmol). The reaction mixture was stirred at room temperature for 1 h, then concentrated to afford a crude product, which was purified by pre-HPLC to afford Example 3 (25.7 mg). calc'd 517.1 [(M+H)⁺], measured 517.0 [(M+H)⁺]. ¹H NMR (400 MHz, DMSO-d₆) δ=11.54 (s, 1H), 11.45 (br d, J=5.4 Hz, 1H), 8.70 (s, 1H), 8.37 (d, J=6.1 Hz, 1H), 7.57 (d, J=7.9 Hz, 1H), 7.39-7.28 (m, 2H), 6.59 (td, J=54.4, 3.5 Hz, 1H), 6.16 (dt, J=3.4, 11.1 Hz, 1H), 4.93-4.81 (m, 2H), 4.03 (s, 3H).

### Example 4

### 5-[3-[(1R)-2,2-difluoro-1-[4-(2,2,2-trifluoroethoxy)-2-pyridyl]ethoxy]-1-methyl-pyrazolo[3,4-c]pyridazin-5-yl]-1H-pyrimidine-2,4-dione

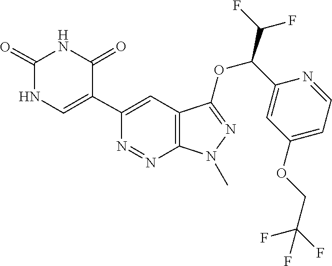 [SMILES: Cn1nc(O[C@H](c2cc(OCC(F)(F)F)ccn2)C(F)F)c2cc(-c3c[nH]c(=O)[nH]c3=O)nnc21]

The titled compound was synthesized according to the following scheme:

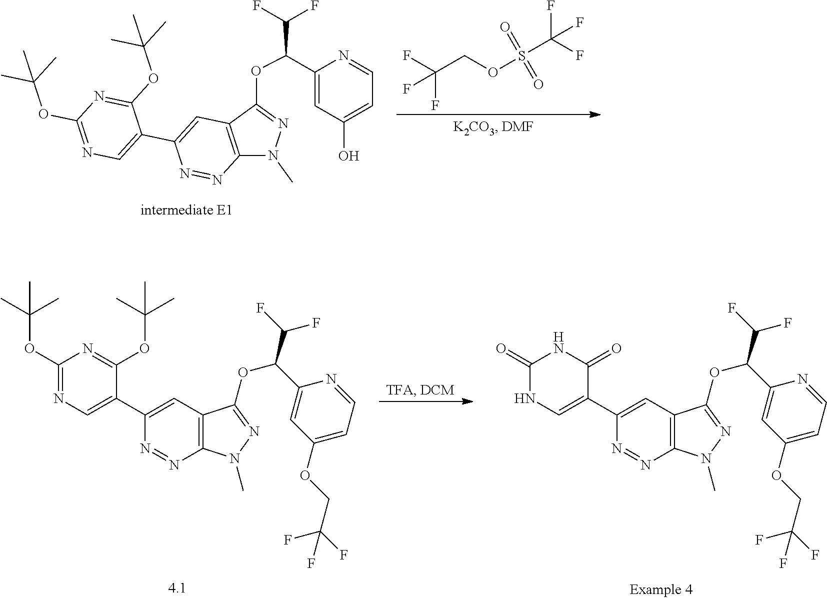

### Step (a): Preparation of 5-(2,4-ditert-butoxypyrimidin-5-yl)-3-[(1R)-2,2-difluoro-1-[4-(2,2,2-trifluoroethoxy)-2-pyridyl]ethoxy]-1-methyl-pyrazolo[3,4-c]pyridazine (compound 4.1)

A mixture of 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol (intermediate E1, 400 mg, 0.76 mmol), 2,2,2-trifluoroethyl trifluoromethanesulfonate (193 mg, 0.83 mmol), potassium carbonate (209 mg, 1.51 mmol) in DMF (10 mL) was stirred at room temperature for 2 h. The reaction mixture was diluted with water (100 mL), extracted with EA (100 mL) twice. The combined organic layer was washed with brine, dried over Na₂SO₄, filtered and concentrated. The residue was purified by silica gel column chromatography (eluted with EA in PE from 0 to 40%) to afford to afford compound 4.1 (360 mg). MS: calc'd 612.2 [(M+H)⁺]; measured 612.3 [(M+H)⁺].

### Step (b): Preparation of 5-[3-[(1R)-2,2-difluoro-1-[4-(2,2,2-trifluoroethoxy)-2-pyridyl]ethoxy]-1-methyl-pyrazolo[3,4-c]pyridazin-5-yl]-1H-pyrimidine-2,4-dione (Example 4)

To a solution of 5-(2,4-ditert-butoxypyrimidin-5-yl)-3-[(1R)-2,2-difluoro-1-[4-(2,2,2-trifluoroethoxy)-2-pyridyl]ethoxy]-1-methyl-pyrazolo[3,4-c]pyridazine (compound 4.1, 176 mg, 0.29 mmol) in DCM (6 mL) was added TFA (0.2 mL). The reaction mixture was stirred at room temperature for 1 h, then concentrated to afford a crude product, which was purified by pre-HPLC to afford Example 4 (37.9 mg). MS: calc'd 500.1 [(M+H)⁺]; measured 500.1 [(M+H)⁺]. ¹H NMR (400 MHz, DMSO-d₆) δ=11.54 (s, 1H), 11.45 (brd, J=4.8 Hz, 1H), 8.70 (d, J=2.0 Hz, 1H), 8.53 (d, J=5.8 Hz, 1H), 8.37 (d, J=6.3 Hz, 1H), 7.36 (s, 1H), 7.16 (dd, J=5.8, 2.5 Hz, 1H), 6.69 (td, J=54.3, 3.8 Hz, 1H), 6.17 (ddd, J=14.4, 8.1, 3.5 Hz, 1H), 4.93 (q, J=8.8 Hz, 2H), 4.02 (s, 3H).

### Example 5

### 5-[3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazin-5-yl]-1H-pyrimidine-2,4-dione

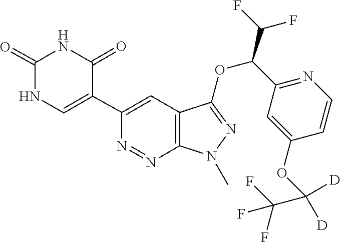 [SMILES: [2H]C([2H])(Oc1ccnc([C@@H](Oc2nn(C)c3nnc(-c4c[nH]c(=O)[nH]c4=O)cc23)C(F)F)c1)C(F)(F)F]

The titled compound was synthesized according to the following scheme:

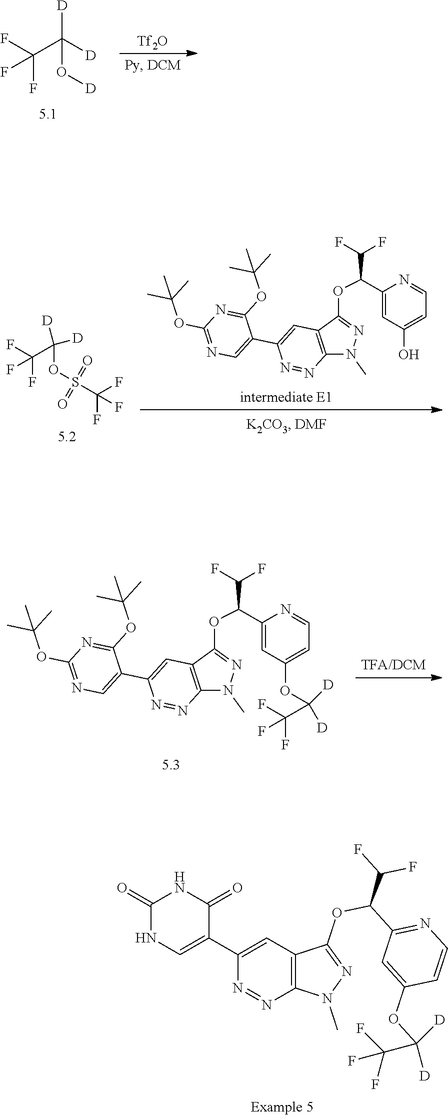

### Step (a): Preparation of (1,1-dideuterio-2,2,2-trifluoro-ethyl) trifluoromethanesulfonate (Compound 5.2)

To a solution of 1,1-dideuterio-1-deuteriooxy-2,2,2-trifluoro-ethane (compound 5.1, 1.19 g, 11.6 mmol) and pyridine (0.98 mL, 12.1 mmol) in DCM (20 mL) was added dropwise trifluoromethanesulfonic anhydride (3.26 g, 11.6 mmol) at 0° C. The resultant mixture was stirred at room temperature for 1 h, then washed with water (20 mL) twice, dried over anhydrous Na₂SO₄ and filtered. The filtrate (a solution of compound 5.2 in DCM) was used as is in the next step directly.

### Step (b): Preparation of 5-(2,4-ditert-butoxypyrimidin-5-yl)-3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazine (compound 5.3)

A mixture of 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol (intermediate E1, 160 mg, 0.300 mmol), (1,1-dideuterio-2,2,2-trifluoro-ethyl) trifluoromethanesulfonate (compound 5.2, 4 mL, ~0.6 M solution in DCM) and potassium carbonate (83.5 mg, 0.600 mmol) in DMF (10 mL) was stirred at room temperature for 16 h. The reaction mixture was diluted with water (100 mL), extracted with EA (100 mL) twice. The combined organic layer was washed with brine, dried over Na₂SO₄, filtered and concentrated. The residue was purified by silica gel column chromatography (eluted with 0 to 40% EA in PE) to afford compound 5.3 (180 mg). MS: calc'd 614.2 [(M+H)⁺]; measured 614.3 [(M+H)⁺].

### Step (c): Preparation of 5-[3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazin-5-yl]-1H-pyrimidine-2,4-dione (Example 5)

To a solution of 5-(2,4-ditert-butoxypyrimidin-5-yl)-3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazine (compound 5.3, 180 mg, 0.29 mmol) in DCM (6 mL) was added TFA (0.2 mL). The reaction mixture was stirred at room temperature for 0.5 h, then concentrated to afford a crude product, which was purified by pre-HPLC to afford Example 5 (35 mg). MS: calc'd 502.1 [(M+H)⁺]; measured 502.1 [(M+H)⁺]. ¹H NMR (400 MHz, DMSO-d₆) δ=11.55 (s, 1H), 11.45 (brd, J=4.8 Hz, 1H), 8.71 (d, J=2.0 Hz, 1H), 8.54 (d, J=5.8 Hz, 1H), 8.37 (dd, J=6.1, 1.4 Hz, 1H), 7.36 (s, 1H), 7.16 (dd, J=5.8, 2.5 Hz, 1H), 6.70 (td, J=53.8, 3.8 Hz, 1H), 6.18 (ddd, J=14.4, 8.1, 3.5 Hz, 1H), 4.02 (s, 3H).

### Example 6

### 5-[3-[(1R)-2,2-difluoro-1-[4-(2,2,2-trifluoroethoxy)-2-pyridyl]ethoxy]-1-(trideuteriomethyl)pyrazolo[3,4-c]pyridazin-5-yl]-1H-pyrimidine-2,4-dione

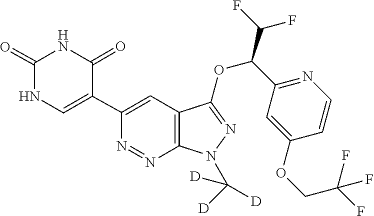 [SMILES: [2H]C([2H])([2H])n1nc(O[C@H](c2cc(OCC(F)(F)F)ccn2)C(F)F)c2cc(-c3c[nH]c(=O)[nH]c3=O)nnc21]

Example 6 was prepared in analogy to Example 4, by replacing 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol (intermediate E1) with 2-[(1R)-1-[5-(2,4-ditert-butoxypyrimidin-5-yl)-1-(trideuteriomethyl)pyrazolo[3,4-c]pyridazin-3-yl]oxy-2,2-difluoro-ethyl]pyridin-4-ol (intermediate E2) in step (a). calc'd 503.1 [(M+H)⁺]; measured 503.1 [(M+H)⁺]. ¹H NMR (400 MHz, DMSO-d₆) δ=11.07 (br s, 2H), 8.71 (s, 1H), 8.54 (d, J=5.5 Hz, 1H), 8.38 (s, 1H), 7.36 (d, J=2.3 Hz, 1H), 7.17 (dd, J=5.6, 2.4 Hz, 1H), 6.70 (td, J=54.3, 4.3 Hz, 1H), 6.18 (ddd, J=14.5, 7.9, 3.1 Hz, 1H), 4.93 (q, J=8.7 Hz, 2H).

### Example 7

### 5-[3-[(1R)-1-[4-(2,2-difluoroethoxy)-2-pyridyl]-2,2-difluoro-ethoxy]-1-methyl-pyrazolo[3,4-c]pyridazin-5-yl]-1H-pyrimidine-2,4-dione

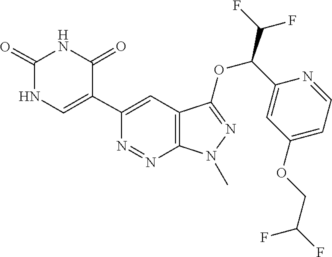 [SMILES: Cn1nc(O[C@H](c2cc(OCC(F)F)ccn2)C(F)F)c2cc(-c3c[nH]c(=O)[nH]c3=O)nnc21]

Example 7 was prepared in analogy to Example 4, by replacing 2,2,2-trifluoroethyl trifluoromethanesulfonate with 2-bromo-1,1-difluoroethane in step (a). MS: calc'd 482.1 [(M+H)⁺]; measured 482.2 [(M+H)⁺]. ¹H NMR (400 MHz, DMSO-d₆) δ=11.55 (s, 1H), 11.45 (brd, J=4.8 Hz, 1H), 8.71 (s, 1H), 8.50 (d, J=5.5 Hz, 1H), 8.37 (d, J=6.3 Hz, 1H), 7.30 (d, J=2.5 Hz, 1H), 7.12 (dd, J=5.8, 2.5 Hz, 1H), 6.70 (td, J=53.5, 3.8 Hz, 1H), 6.39 (td, J=54.3, 3.8 Hz, 1H), 6.17 (ddd, J=14.2, 7.8, 3.3 Hz, 1H), 4.46 (td, J=14.7, 3.1 Hz, 2H), 4.03 (s, 3H).

### Example 8A and Example 8B

### 5-[3-[(1R)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione and 5-[3-[(1S)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione

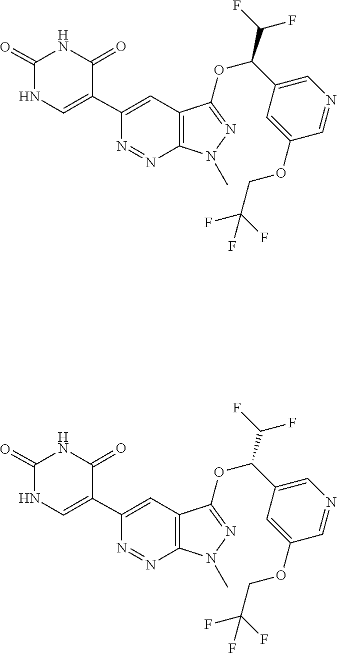 [SMILES: Cn1nc(O[C@H](c2cncc(OCC(F)(F)F)c2)C(F)F)c2cc(-c3c[nH]c(=O)[nH]c3=O)nnc21 ; Cn1nc(O[C@@H](c2cncc(OCC(F)(F)F)c2)C(F)F)c2cc(-c3c[nH]c(=O)[nH]c3=O)nnc21]

The titled compound was synthesized according to the following scheme:

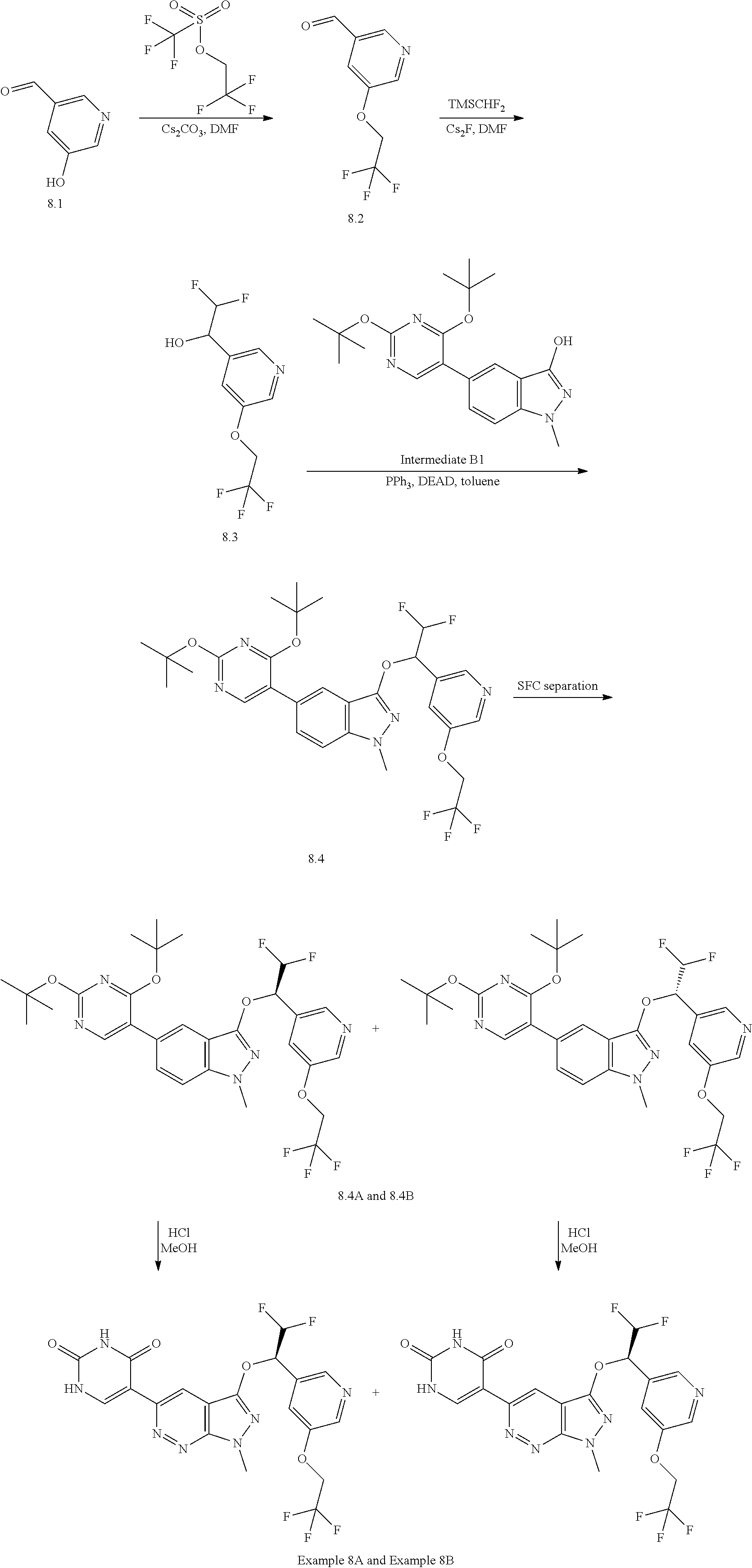

### Step (a): Preparation of 5-(2,2,2-trifluoroethoxy)pyridine-3-carbaldehyde (Compound 8.2)

To a solution of 5-hydroxypyridine-3-carbaldehyde (compound 8.1, 1 g, 8.1 mmol) and 2,2,2-trifluoroethyl trifluoromethanesulfonate (2.3 g, 1.4 mL, 9.8 mmol) in DMF (30 mL) was added cesium carbonate (4.0 g, 974.9 μL, 12.2 mmol). The resultant mixture was stirred at 20° C. for 16 h, then quenched with H₂O (100 mL) and extracted with EA (20 mL) for three times. The combined organic layer was washed with H₂O (30 mL) for three times, dried over Na₂SO₄, filtered and concentrated. The residue was purified by silica gel chromatography (eluted with PE/EA from 0/1 to 1/3) to afford compound 8.2 (1.76 g). calc'd 206.0 [(M+H)⁺], measured 206.0 [(M+H)⁺].

### Step (b): Preparation of 2,2-difluoro-1-[5-(2,2,2-trifluoroethoxy)-3-pyridyl]ethanol (Compound 8.3)

To a solution of 5-(2,2,2-trifluoroethoxy)pyridine-3-carbaldehyde (compound 8.2, 1 g, 4.87 mmol) and (difluoromethyl)trimethyl-silane (1.2 g, 1.4 mL, 9.8 mmol) in DMF (8 mL) was added cesium fluoride (148.1 mg, 975.0 μmol). The resultant mixture was stirred at room temperature for 16 h under nitrogen. The reaction was quenched with H₂O (50 mL), and extracted with EA (50 mL) for three times. The combined organic layer was washed with H₂O (30 mL) for three times, dried over Na₂SO₄, filtered and concentrated. The residue was purified by silica gel chromatography (eluted with DCM/methanol from 0/1 to 30/1) to afford compound 8.3 (1.76 g). calc'd 258.0 [(M+H)⁺], measured 257.9 [(M+H)⁺].

### Step (c): Preparation of 5-(2,4-ditert-butoxypyrimidin-5-yl)-3-[2,2-difluoro-1-[5-(2,2,2-trifluoroethoxy)-3-pyridyl]ethoxy]-1-methyl-pyrazolo[3,4-c]pyridazine (compound 8.4)

To the suspension of 2,2-difluoro-1-[5-(2,2,2-trifluoroethoxy)-3-pyridyl]ethanol (compound 8.3, 298.3 mg, 1.2 mmol), 5-(2,4-ditert-butoxypyrimidin-5-yl)-1-methyl-pyrazolo[3,4-c]pyridazin-3-ol (intermediate B1, 360.0 mg, 966.7 μmol) and triphenylphosphine (316.9 mg, 1.2 mmol) in toluene (8 mL) was added DEAD (210.4 mg, 190.3 μL, 1.2 mmol) dropwise at room temperature. The resultant mixture was stirred at 60° C. for 1 h under nitrogen. After being cooled to room temperature, the reaction mixture was diluted with water (50 mL), extracted with EA (30 mL) for three times. The combined organic layer was washed with brine, dried over Na₂SO₄, filtered and concentrated. The residue was purified by silica gel chromatography (eluted with PE/EA from 0/1 to 1/2) to afford compound 8.4 (160 mg). calc'd 612.2 [(M+H)⁺], measured 612.2 [(M+H)⁺].

### Step (d): Preparation of 5-(2,4-ditert-butoxypyrimidin-5-yl)-3-[(1R)-2,2-difluoro-1-[5-(2,2,2-trifluoroethoxy)-3-pyridyl]ethoxy]-1-methyl-pyrazolo[3,4-c]pyridazine and 5-(2,4-ditert-butoxypyrimidin-5-yl)-3-[(1S)-2,2-difluoro-1-[5-(2,2,2-trifluoroethoxy)-3-pyridyl]ethoxy]-1-methyl-pyrazolo[3,4-c]pyridazine (Compound 8.4A and Compound 8.4B)

Compound 8.4 (700.0 mg) was resolved by SFC to give two single isomers: compound 8.4A (faster eluting, 60.0 mg) MS: calc'd 612.2 [(M+H)⁺]; measured 612.2 [(M+H)⁺].; and compound 8.4B (slower eluting, 50.0 mg) MS: calc'd 612.2 [(M+H)⁺]; measured 612.2 [(M+H)⁺]. SFC conditions: Column: (S,S) whelk-ol, 250×30 mm I.D., 5 μm, mobile phase: A for CO₂ and B for ethanol (0.1% NH₃H₂O), gradient: B 30%, flow rate: 80 mL/min, back pressure: 100 bar, Column temperature: 35° C.

### Step (e):preparation of 5-[3-[(1R)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione and 5-[3-[(1S)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione (Example 8A and Example 8B)

To a solution of compound 8.4A (60 mg, 98.1 μmol) in methanol (500 μL) was added 2 N HCl (aq.) (245.3 μL, 490.6 μmol). The reaction mixture was stirred at room temperature for 1 h, then concentrated to afford a crude product, which was purified by pre-HPLC to afford Example 8A (16.0 mg). calc'd 500.1 [(M+H)⁺], measured 500.0 [(M+H)⁺]. ¹H NMR (400 MHz, DMSO-d₆) δ=11.54 (d, J=1.4 Hz, 1H), 11.45 (br d, J=6.1 Hz, 1H), 8.71 (s, 1H), 8.53 (s, 1H), 8.46 (d, J=2.8 Hz, 1H), 8.36 (d, J=6.1 Hz, 1H), 7.79 (br s, 1H), 6.68 (td, J=54.03, 3.13 Hz, 1H), 6.29 (dt, J=3.2, 11.4 Hz, 1H), 4.92 (q, J=8.8 Hz, 2H), 4.03 (s, 3H).

To a solution of compound 8.4B (50 mg, 81.8 μmol) in methanol (400 μL) was added 2 N HCl (aq.) (204.4 μL, 409.0 μmol). The reaction mixture was stirred at room temperature for 1 h, then concentrated to afford a crude product, which was purified by pre-HPLC to afford Example 8B (23.0 mg). calc'd 500.1 [(M+H)⁺], measured 500.0 [(M+H)⁺]. ¹H NMR (400 MHz, DMSO-d₆) δ=11.54 (d, J=1.5 Hz, 1H), 11.46 (br d, J=5.5 Hz, 1H), 8.71 (s, 1H), 8.54 (s, 1H), 8.48 (d, J=2.6 Hz, 1H), 8.37 (d, J=6.1 Hz, 1H), 7.81 (d, J=1.8 Hz, 1H), 6.68 (td, J=54.16, 3.00 Hz, 1H), 6.30 (dt, J=3.2, 11.3 Hz, 1H), 4.92 (q, J=8.8 Hz, 2H), 4.03 (s, 3H).

### Example 9A and Example 9B

### 5-[3-[(1R)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione and 5-[3-[(1S)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione

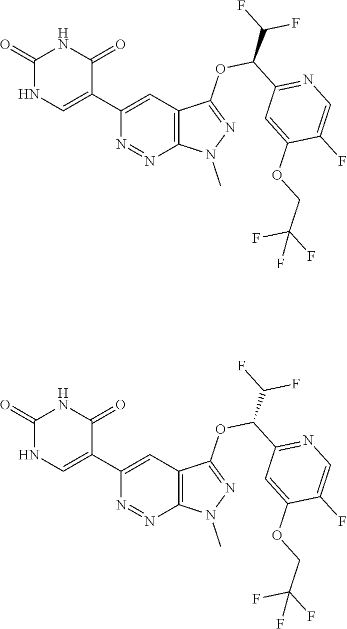 [SMILES: Cn1nc(O[C@H](c2cc(OCC(F)(F)F)c(F)cn2)C(F)F)c2cc(-c3c[nH]c(=O)[nH]c3=O)nnc21 ; Cn1nc(O[C@@H](c2cc(OCC(F)(F)F)c(F)cn2)C(F)F)c2cc(-c3c[nH]c(=O)[nH]c3=O)nnc21]

The titled compound was synthesized according to the following scheme:

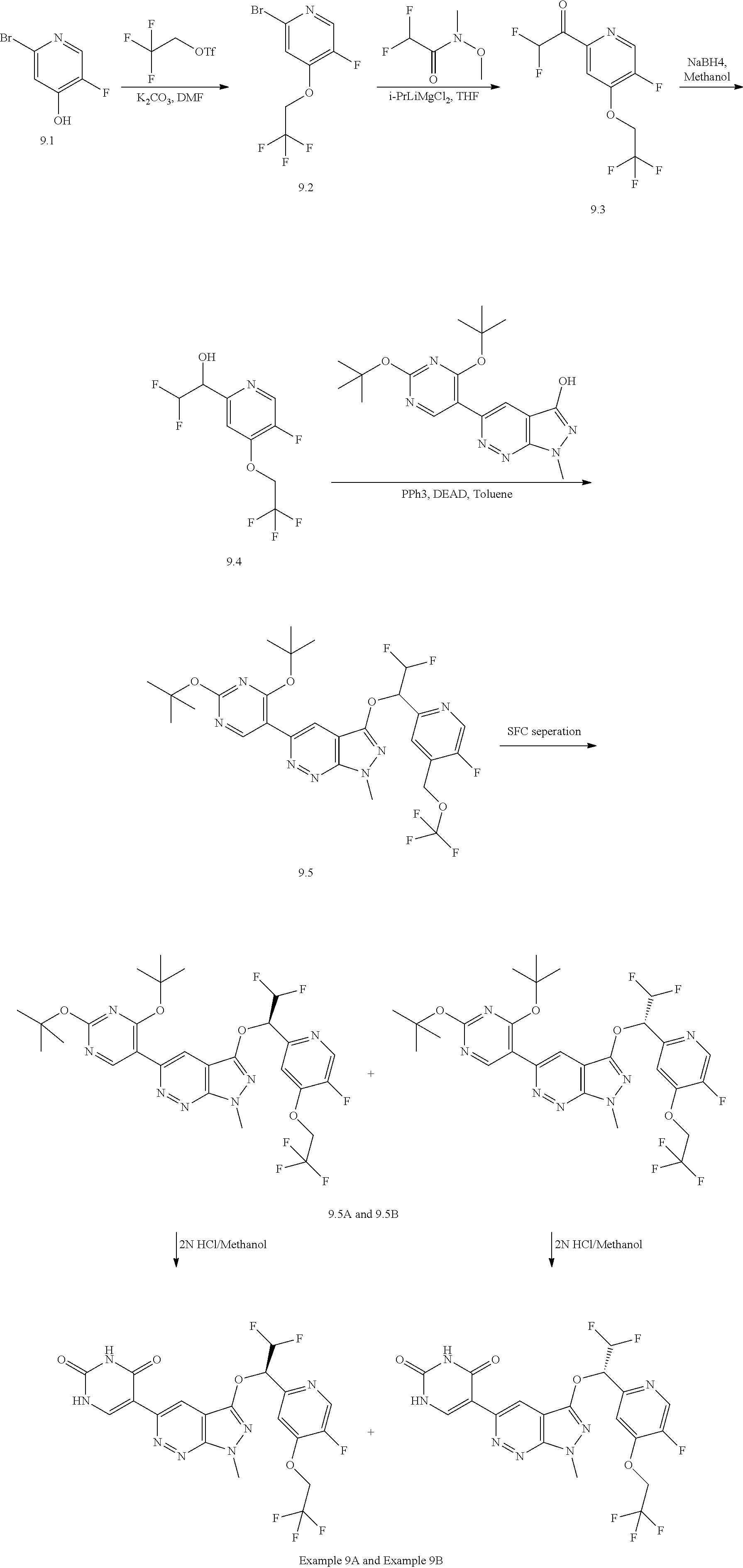

### Step (a): Preparation of 2-bromo-5-fluoro-4-(2,2,2-trifluoroethoxy)pyridine (Compound 9.2)

A mixture of 2,2,2-trifluoroethyl trifluoromethanesulfonate (1.81 g, 7.81 mmol), 2-bromo-5-fluoro-pyridin-4-ol (compound 9.1, 0.50 g, 2.6 mmol) and potassium carbonate (0.72 g, 5.21 mmol) in DMF (10 mL) was stirred at room temperature for 3 h. The reaction was quenched with water (100 mL), and extracted with EA (150 mL) for three times. The combined organic layer was washed with brine, dried over Na₂SO₄, filtered and concentrated. The residue was purified by silica gel column chromatography (eluted with 5 to 20% EA in PE) to afford compound 9.2 (617 mg). MS: calc'd 273.9, 275.9 [(M+H)⁺]; measured 274.0, 274.0 [(M+H)⁺]. ¹H NMR (400 MHz, Chloroform-d) δ 8.23 (d, J=2.6 Hz, 1H), 7.11 (d, J=6.0 Hz, 1H), 4.52 (q, J=7.7 Hz, 2H).

### Step (b): Preparation of 2,2-difluoro-1-[5-fluoro-4-(2,2,2-trifluoroethoxy)-2-pyridyl]ethanone (compound 9.3)

A solution of (CH₃)₂CHMgCl·LiCl (1.3 M in THF, 5.03 mL, 6.54 mmol) was added dropwise to a solution of 2-bromo-5-fluoro-4-(2,2,2-trifluoroethoxy)pyridine (compound 9.2, 597 mg, 2.18 mmol) in dry THF (8 mL) at room temperature. After the reaction mixture was stirred for 2 h at room temperature, a solution of 2,2-difluoro-N-methoxy-N-methylacetamide (909.19 mg, 6.54 mmol, 3.0 eq) in dry THF (4 mL) as added dropwise. The resultant mixture was stirred at room temperature for another 2 h, then quenched by saturated aqueous ammonium chloride (100 mL), and extracted with EA (200 mL) for three times. The combined organic layer was dried over Na₂SO₄, filtered and concentrated. The residue was purified by silica gel column chromatography (eluted with 5 to 20% EA in PE) to afford compound 9.3 (503 mg). MS: calc'd 274.0 [(M+H)⁺]; measured 292.0 [(M+H+18)⁺].

### Step (c): Preparation of 2,2-difluoro-1-[5-fluoro-4-(2,2,2-trifluoroethoxy)-2-pyridyl]ethanol (compound 9.4)

To a solution of 2,2-difluoro-1-[5-fluoro-4-(2,2,2-trifluoroethoxy)-2-pyridyl]ethanone (compound 9.3, 200.0 mg, 0.73 mmol) in MeOH (2.5 mL) was added NaBH₄ (55.5 mg, 1.46 mmol). The resultant mixture was stirred at 0° C. for 2 h, then diluted with saturated aqueous NaHCO₃ (20 mL), and extracted with EA (20 mL) for three times. The combined organic layer was washed with brine, dried over Na₂SO₄, filtered and concentrated in vacuo. The residue was purified by flash chromatography (silica gel, 20 g, 0% to 100% EA in PE) to afford compound 9.4 (170.0 mg). MS: calc'd 276.0 [(M+H)⁺]; measured 276.0 [(M+H)⁺].

### Step (d): Preparation of 5-[3-[(1R)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione and 5-[3-[(1S)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione (Example 9A and 9B)

Example 9A and Example 9B was prepared in analogy to Example 8A and Example 8B, by replacing 2,2-difluoro-1-[5-(2,2,2-trifluoroethoxy)-3-pyridyl]ethanol (compound 8.3) with 2,2-difluoro-1-[5-fluoro-4-(2,2,2-trifluoroethoxy)-2-pyridyl]ethanol (compound 9.4) in step (c).

Example 9A MS: calc'd 518.1 [(M+H)⁺]; measured 518.1 [(M+H)⁺]. ¹H NMR (400 MHz, DMSO-d₆) δ=11.54 (d, J=1.6 Hz, 1H), 11.46 (brd, J=6.1 Hz, 1H), 8.71 (s, 1H), 8.61 (d, J=2.8 Hz, 1H), 8.38 (d, J=6.1 Hz, 1H), 7.69 (d, J=6.8 Hz, 1H), 6.68 (dt, J=4.4, 53.4 Hz, 1H), 6.15 (ddd, J=3.6, 8.9, 13.1 Hz, 1H), 5.10-5.01 (m, 2H), 4.03 (s, 3H).

Example 9B MS: calc'd 518.1 [(M+H)⁺]; measured 518.1 [(M+H)⁺]. ¹H NMR (400 MHz, DMSO-d₆) δ=11.54 (d, J=1.6 Hz, 1H), 11.46 (brd, J=5.3 Hz, 1H), 8.71 (s, 1H), 8.61 (d, J=2.9 Hz, 1H), 8.38 (d, J=6.1 Hz, 1H), 7.69 (d, J=6.9 Hz, 1H), 6.68 (dt, J=3.4, 53.5 Hz, 1H), 6.21-6.09 (m, 1H), 5.05-5.01 (m, 2H), 4.03 (s, 3H).

### Example 10

### 5-[3-[(1R)-2,2-difluoro-1-[2-(2,2,2-trifluoroethoxy)-4-pyridyl]ethoxy]-1-methyl-pyrazolo[3,4-c]pyridazin-5-yl]-1H-pyrimidine-2,4-dione

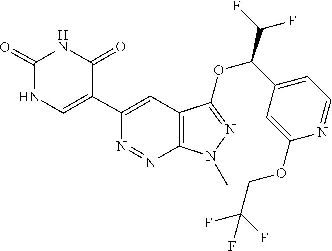 [SMILES: Cn1nc(O[C@H](c2ccnc(OCC(F)(F)F)c2)C(F)F)c2cc(-c3c[nH]c(=O)[nH]c3=O)nnc21]

The titled compound was synthesized according to the following scheme:

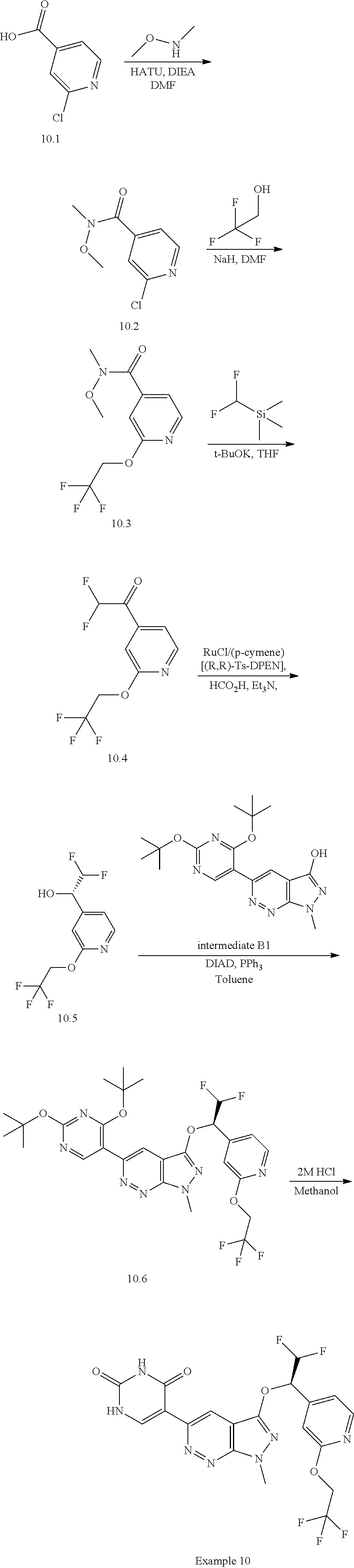

### Step (a): Preparation of 2-chloro-N-methoxy-N-methyl-pyridine-4-carboxamide (Compound 10.2)

To a suspension of 2-chloropyridine-4-carboxylic acid (compound 10.1, 10 g, 63.47 mmol) and Et₃N (17.69 mL, 126.94 mmol) in DCM (100 mL) was added HATU (26.55 g, 69.82 mmol). After the reaction mixture was stirred at room temperature for 30 min, N,O-dimethylhydroxylamine hydrochloride (6.81 g, 69.82 mmol) was added, and the resultant mixture was stirred at room temperature for another 1 h, then quenched with saturated aqueous NaHCO₃ (300 mL), and extracted with DCM (100 mL) for three times. The combined organic layer was dried over Na₂SO₄ and concentrated. The residue was purified by flash chromatography (silica gel, 220 g, eluted with 10% to 40% EA in PE) to afford compound 10.2 (12.3 g). MS: calc'd 201.1 [(M+H)⁺]; measured 201.1 [(M+H)⁺].

### Step (b): Preparation of N-methoxy-N-methyl-2-(2,2,2-trifluoroethoxy)pyridine-4-carboxamide (compound 10.3)

To a solution of 2,2,2-trifluoroethanol (1.39 g, 13.9 mmol) in DMF (20 mL) was added NaH (60% dispersion in mineral oil, 598.2 mg, 14.95 mmol) at 0° C. After the reaction mixture was stirred at the same temperature for 0.5 h, 2-chloro-N-methoxy-N-methyl-pyridine-4-carboxamide (compound 10.2, 2 g, 9.97 mmol) was added, and the resultant mixture was stirred at 60° C. for 0.5 h. After being cooled to room temperature, the reaction mixture was diluted with saturated aqueous NaCl (50 mL), and extracted with EA (30 mL) for three times. The combined organic layer was washed with brine, dried over Na₂SO₄, filtered and concentrated. The residue was purified by flash chromatography (silica gel, 45 g, eluted with 10% to 35% EA in PE) to afford compound 10.3 (982.8 mg). MS: calc'd 265.1 [(M+H)⁺]; measured 265.1 [(M+H)⁺].

### Step (c): Preparation of 2,2-difluoro-1-[2-(2,2,2-trifluoroethoxy)-4-pyridyl]ethanone (Compound 10.4)

To a solution N-methoxy-N-methyl-2-(2,2,2-trifluoroethoxy)pyridine-4-carboxamide (compound 10.3, 708.3 mg, 2.68 mmol) and difluoromethyltrimethylsilane (613.9 mg, 4.94 mmol) in THF (10 mL) was added potassium tert-butoxide (330.9 mg, 2.95 mmol) at room temperature. The resultant mixture was stirred at the same temperature for another 2 h. The reaction mixture was quenched with saturated aqueous NaCl (50 mL), and extracted with EA (20 mL) for three times. The combined organic layer was dried over Na₂SO₄, filtered and concentrated. The residue was purified by flash chromatography (silica gel, 24 g, eluted with 0% to 30% EA in PE) to afford compound 10.4 (397.2 mg). MS: calc'd 256.0 [(M+H)⁺]; measured 256.0 [(M+H)⁺].

### Step (d): Preparation of (1S)-2,2-difluoro-1-[2-(2,2,2-trifluoroethoxy)-4-pyridyl]ethanol (compound 10.5)

To a solution of 2,2-difluoro-1-[2-(2,2,2-trifluoroethoxy)-4-pyridyl]ethanone (compound 10.4, 4.79 g, 18.77 mmol) in DCM (20 mL) was added formic acid (7 mL, 182.49 mmol) and triethylamine (10 mL, 71.75 mmol) at −20° C. Then RuCl(p-cymene)[(R,R)-Ts-DPEN](477.8 mg, 750.9 μmol) was added, and the mixture was stirred at room temperature for 3 hr. The reaction mixture was diluted with saturated aqueous NaHCO₃ (200 mL), and extracted with DCM (100 mL) twice. The combined organic layer was washed with brine, dried over Na₂SO₄, filtered and concentrated. The residue was purified by flash chromatography (silica gel, 45 g, eluted with 10% to 35% EA in PE) to afford compound 10.5 (4.3 g). MS: calc'd 258.0 [(M+H)⁺]; measured 258.0 [(M+H)⁺].

### Step (e): Preparation of 5-[3-[(1R)-2,2-difluoro-1-[2-(2,2,2-trifluoroethoxy)-4-pyridyl]ethoxy]-1-methyl-pyrazolo[3,4-c]pyridazin-5-yl]-1H-pyrimidine-2,4-dione

(Example 10) Example 10 was prepared in analogy to Example 2, by replacing (1S)-1-[2-(cyclopropoxymethyl)-4-pyridyl]-2,2-difluoro-ethanol (compound 2.6) with (1S)-2,2-difluoro-1-[2-(2,2,2-trifluoroethoxy)-4-pyridyl]ethanol (compound 10.5) in step (f). MS: calc'd 500.1 [(M+H)⁺]; measured 500.0 [(M+H)⁺]. ¹H NMR (400 MHz, DMSO-d₆) δ=11.55 (d, J=1.6 Hz, 1H), 11.49-11.40 (m, 1H), 8.72 (s, 1H), 8.37 (d, J=6.1 Hz, 1H), 8.26 (d, J=5.5 Hz, 1H), 7.37-7.31 (m, 1H), 7.21 (s, 1H), 6.64 (dt, J=2.9, 53.7 Hz, 1H), 6.30 (dt, J=2.5, 11.3 Hz, 1H), 5.07-4.94 (m, 2H), 4.02 (s, 3H).

### Example 11A and Example 11B

### 5-[3-[(1S)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione and 5-[3-[(1R)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione

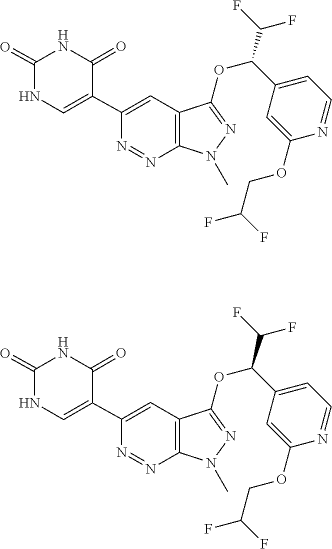 [SMILES: Cn1nc(O[C@@H](c2ccnc(OCC(F)F)c2)C(F)F)c2cc(-c3c[nH]c(=O)[nH]c3=O)nnc21 ; Cn1nc(O[C@H](c2ccnc(OCC(F)F)c2)C(F)F)c2cc(-c3c[nH]c(=O)[nH]c3=O)nnc21]

The titled compound was synthesized according to the following scheme:

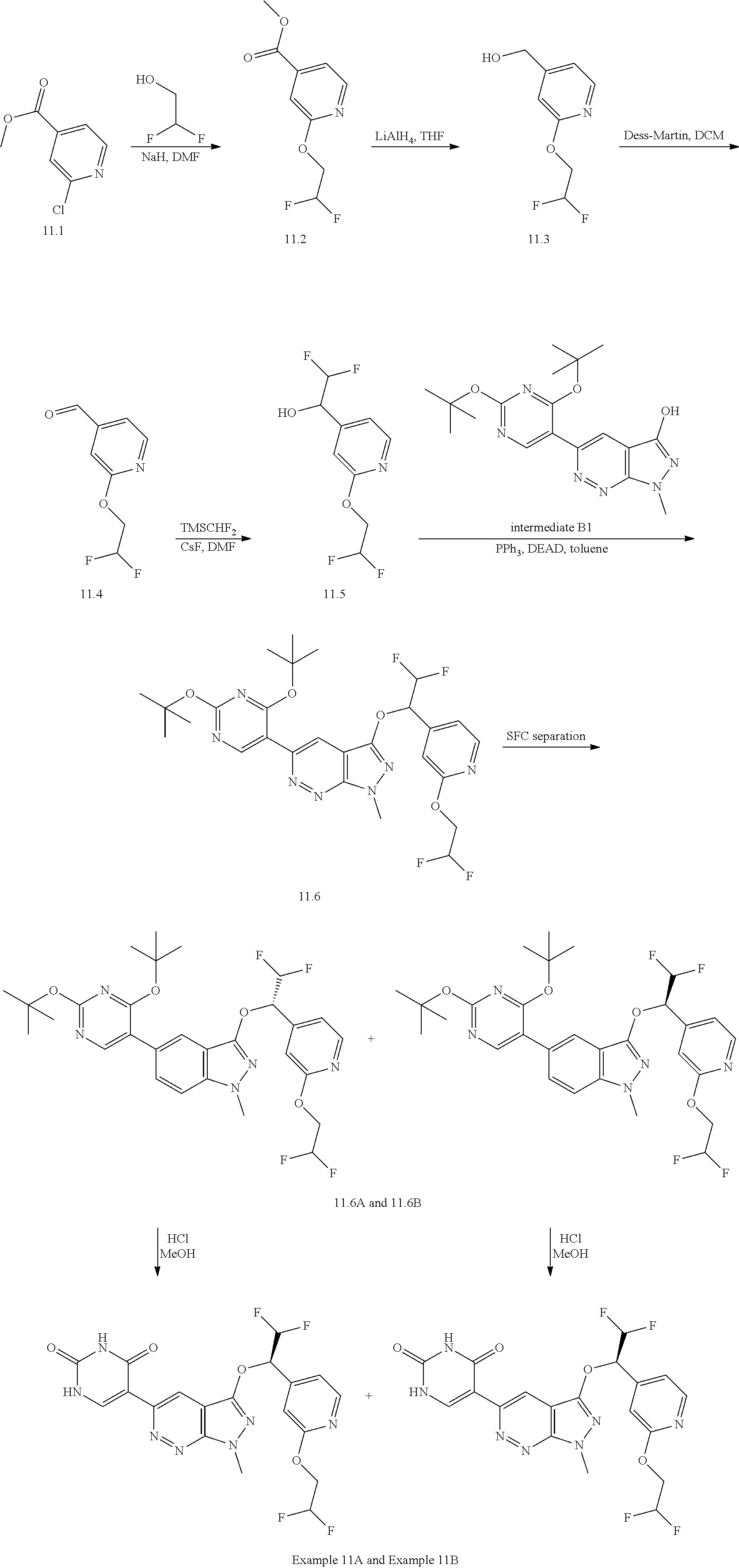

### Step (a): Preparation of methyl 2-(2,2-difluoroethoxy)pyridine-4-carboxylate (Compound 11.2)

To a solution of 2,2-difluoroethanol (3.7 g, 44.9 mmol) in DMF (35 mL) was added sodium hydride (60% dispersion in mineral oil, 2.61 g, 65.3 mmol) at 0° C. After the reaction mixture was stirred at 0° C. for 30 min, a solution of methyl 2-chloropyridine-4-carboxylate (compound 11.1, 7 g, 40.8 mmol) in DMF (10 mL) was added, and the resultant mixture was stirred at 20° C. for 16 h, then quenched with H₂O (200 mL) and extracted with EA (60 mL) for three times. The combined organic layer was washed with brine, dried over Na₂SO₄ and concentrated. The residue was purified by silica gel chromatography (eluted with PE/EA from 0/1 to 20/1) to afford compound 11.2 (5.8 g). calc'd 218.1 [(M+H)⁺], measured 218.0 [(M+H)⁺].

### Step (b): Preparation [2-(2,2-difluoroethoxy)-4-pyridyl]methanol (compound 11.3)

To a solution of methyl 2-(2,2-difluoroethoxy)pyridine-4-carboxylate (compound 11.2, 5.8 g, 26.7 mmol) in 20 mL of THF was added a suspension of LiAlH₄ (709.5 mg, 18.7 mmol) in tetrahydrofuran (50 mL) dropwise at 0° C. The resultant mixture was stirred at the same temperature for 30 min, then diluted with ethyl acetate (60 mL), and 40 g of Na₂SO₄ was added while the mixture was stirred vigorously, followed by addition of 1 mL of H₂O. The resultant mixture was filtered through a celite, washed with ethyl acetate (60 mL) twice, and the filtrate was concentrated. The residue was purified by silica gel chromatography (eluted with PE/EA from 10/1 to 1/1) to afford compound 11.3 (3.5 g). calc'd 190.1 [(M+H)⁺], measured 190.0 [(M+H)⁺].

### Step (c): Preparation of 2-(2,2-difluoroethoxy)pyridine-4-carbaldehyde (compound 11.4)

To a solution of [2-(2,2-difluoroethoxy)-4-pyridyl]methanol(compound 11.3, 3.5 g, 18.5 mmol) in DCM (40 mL) was added DMP (15.7 g, 37.0 mmol) in portions. The resultant mixture was stirred at 0° C. for 1 h. And then the reaction was quenched with saturated aqueous NaHCO₃ and saturated aqueous Na₂S₂O₃ (150 mL each). After being stirred at room temperature for 0.5 h, the mixture was extracted with DCM (150 mL) for three times. The combined organic layer was concentrated. The residue was purified by silica gel chromatography (eluted with PE/EA from 0/1 to 50/1) to afford compound 11.4 (3.3 g). calc'd 188.0 [(M+H)⁺], measured 188.0 [(M+H)⁺].

### Step (d): Preparation of 1-[2-(2,2-difluoroethoxy)-4-pyridyl]-2,2-difluoro-ethanol (Compound 11.5)

To a solution of 2-(2,2-difluoroethoxy)pyridine-4-carbaldehyde (compound 11.4, 3.3 g, 17.4 mmol) and difluoromethyl-trimethyl-silane (4.3 g, 34.8 mmol) in DMF (10 mL) was added TBAF (1 M in THF, 8.7 mL, 8.7 mmol) at 0° C. After stirred at 20° C. for 1 h, the reaction was quenched with H₂O (40 mL), and extracted with EA (60 mL) three times. The combined organic layer was washed with H₂O (50 mL) for seven times, dried over Na₂SO₄, filtered and concentrated. The residue was purified by silica gel chromatography (eluted with PE/EA from 0/1 to 2/1) to afford compound 11.5 (2 g). calc'd 240.1 [(M+H)⁺], measured 239.9 [(M+H)⁺].

### Step (c): Preparation of 5-[3-[(1S)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione and 5-[3-[(1R)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione (Example 11A and Example 11B)

Example 11A and Example 11B was prepared in analogy to Example 8A and Example 8B, by replacing 2,2-difluoro-1-[5-(2,2,2-trifluoroethoxy)-3-pyridyl]ethanol (compound 8.3) with 1-[2-(2,2-difluoroethoxy)-4-pyridyl]-2,2-difluoro-ethanol (compound 11.5) in step (c).

Example 11A calc'd 482.1 [(M+H)⁺], measured 482.0 [(M+H)⁺]. ¹H NMR (400 MHz, DMSO-d₆) δ=11.55 (s, 1H), 11.43-11.52 (m, 1H), 8.72 (s, 1H), 8.37 (d, J=6.25 Hz, 1H), 8.24 (d, J=5.38 Hz, 1H), 7.28 (d, J=5.25 Hz, 1H), 7.14 (s, 1H), 6.64 (td, J=53.8, 2.8 Hz, 1H), 6.38 (tt, J=54.7, 3.5 Hz, 1H), 6.25-6.33 (m, 1H), 4.56 (td, J=15.01, 3.50 Hz, 2H), 4.02 (s, 3H).

Example 11B calc'd 482.1 [(M+H)⁺], measured 482.0 [(M+H)⁺]. ¹H NMR (400 MHz, DMSO-d₆) δ=11.55 (d, J=1.63 Hz, 1H), 11.42-11.49 (m, 1H), 8.72 (s, 1H),) 8.37 (d, J=6.13 Hz, 1H), 8.23 (d, J=5.38 Hz, 1H), 7.28 (dd, J=5.32, 1.06 Hz, 1H), 7.14 (s, 1H), 6.63 (td, J=53.8, 2.8 Hz, 1H), 6.38 (tt, J=54.7, 3.4 Hz, 1H), 6.25-6.33 (m, 1H), 4.56 (td, J=15.04, 3.44 Hz, 2H), 4.02 (s, 3H).

### Example 12

### 6-[3-[(1R)-2,2-difluoro-1-[2-(2,2,2-trifluoroethoxy)-4-pyridyl]ethoxy]-1-methyl-pyrazolo[3,4-c]pyridazin-5-yl]-2H-1,2,4-triazine-3,5-dione

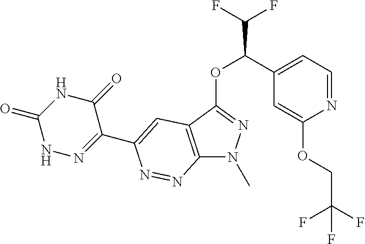 [SMILES: Cn1nc(O[C@H](c2ccnc(OCC(F)(F)F)c2)C(F)F)c2cc(-c3n[nH]c(=O)[nH]c3=O)nnc21]

The titled compound was synthesized according to the following scheme:

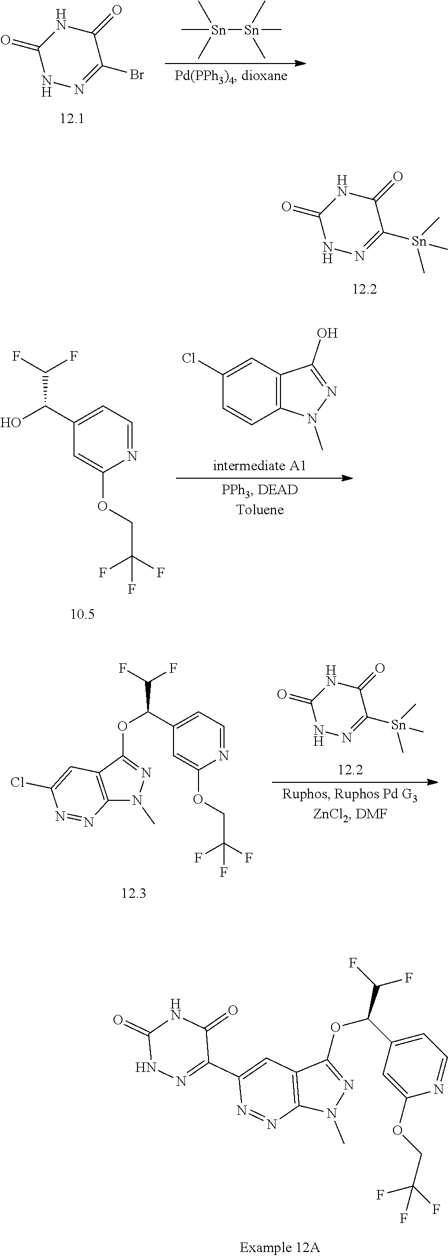

### Step (a): Preparation of 6-trimethylstannyl-2H-1,2,4-triazine-3,5-dione (compound 12.2)

To a solution of 5-bromo-6-azauracil (compound 12.1, 600 mg, 3.13 mmol) in 1,4-dioxane (10 mL) was added 1,1,1,2,2,2-hexamethyldistannane (1024 mg, 3.13 mmol) and Pd(PPh₃)₄ (180 mg, 0.16 mmol). The resultant mixture was stirred for 6 h at 100° C. under nitrogen atmosphere, After being cooled to room temperature, the reaction mixture was diluted with water (50 mL), extracted with EA (100 mL) for three times. The combined organic layer was washed with brine, dried over Na₂SO₄, filtered and concentrated. The residue was purified by flash chromatography (silica gel, eluted with 0% to 60% EA in PE) to afford compound 12.2 (706 mg). ¹H NMR (400 MHz, DMSO-d₆) δ=12.44 (s, 1H), 11.67 (s, 1H), 0.26 (s, 9H).

### Step (b): Preparation of 5-chloro-3-[(1R)-2,2-difluoro-1-[2-(2,2,2-trifluoroethoxy)-4-pyridyl]ethoxy]-1-methyl-pyrazolo[3,4-c]pyridazine (compound 12.3)

To a suspension of (1S)-2,2-difluoro-1-[2-(2,2,2-trifluoroethoxy)-4-pyridyl]ethanol (compound 10.5, 10.3 g, 40.2 mmol) and 5-chloro-1-methyl-pyrazolo[3,4-c]pyridazin-3-ol (intermediate A1, 3.9 g, 21.1 mmol) and triphenylphosphine (6.7 g, 25.4 mmol) in toluene (78 mL) was added DEAD (4.4 g, 4.0 mL, 25.4 mmol) dropwise at 60° C. After being stirred at 60° C. for 0.5 h under nitrogen, the reaction mixture was concentrated, and the residue was purified by silica gel chromatography (eluted with PE/EA from 0/1 to 7/1) to afford compound 12.3 (8.6 g). calc'd 424.1 [(M+H)⁺], measured 424.0 [(M+H)⁺].

### Step (c): Preparation of 6-[3-[(1R)-2,2-difluoro-1-[2-(2,2,2-trifluoroethoxy)-4-pyridyl]ethoxy]-1-methyl-pyrazolo[3,4-c]pyridazin-5-yl]-2H-1,2,4-triazine-3,5-dione (Example 12)

To a degassed vial were added 5-chloro-3-[(1R)-2,2-difluoro-1-[2-(2,2,2-trifluoroethoxy)-4-pyridyl]ethoxy]-1-methyl-pyrazolo[3,4-c]pyridazine (compound 12.3, 4.6 g, 10.86 mmol), 6-trimethylstannyl-2H-1,2,4-triazine-3,5-dione (compound 12.2, 6.0 g, 21.7 mmol), dicyclohexyl[2-(2,6-diisopropoxyphenyl)phenyl]phosphane (253.3 mg, 542.8 μmol), methanesulfonato(2-dicyclohexylphosphino-2′,6′-di-i-propoxy-1,1′-biphenyl)(2′-amino-1,1-biphenyl-2-yl)palladium(ii) (453.9 mg, 542.8 μmol) and anhydrous zinc chloride (2.2 g, 16.3 mmol) in DMF (46 mL). The resultant mixture was stirred at 100° C. for 16 h under nitrogen. After being cooled to room temperature, the reaction mixture was diluted with saturated aqueous KF (50 mL), and stirred for 30 min, and then filtered through a celite. The filtrate was extracted with EA (500 mL) for three times. The combined organic layer was washed with H₂O (300 mL) for three times, dried over Na₂SO₄, filtered and concentrated. The residue was purified by silica gel chromatography (eluted with DCM/MeOH from 0/1 to 12/1), which was further purified by Prep-HPLC to afford example 12 (2.6 g). calc'd 501.1 [(M+H)⁺], measured 501.0 [(M+H)⁺]. ¹H NMR (400 MHz, DMSO-d₆) δ=11.42 (br s, 2H), 8.53 (s, 1H), 8.26 (d, J=5.38 Hz, 1H), 7.34 (dd, J=5.25, 0.88 Hz, 1H), 7.22 (s, 1H), 6.65 (td, J=53.66, 2.75 Hz, 1H), 6.28-6.40 (m, 1H), 4.89-5.08 (m, 2H), 4.06 (s, 3H).

### Example 13A and Example 13B

### 6-[3-[(1R)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione and 6-[3-[(1S)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione

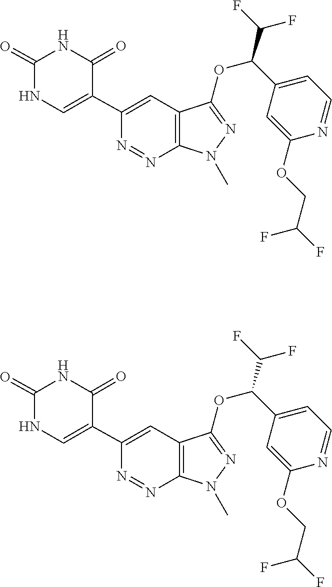 [SMILES: Cn1nc(O[C@H](c2ccnc(OCC(F)F)c2)C(F)F)c2cc(-c3c[nH]c(=O)[nH]c3=O)nnc21 ; Cn1nc(O[C@@H](c2ccnc(OCC(F)F)c2)C(F)F)c2cc(-c3c[nH]c(=O)[nH]c3=O)nnc21]

The titled compounds were synthesized according to the following scheme:

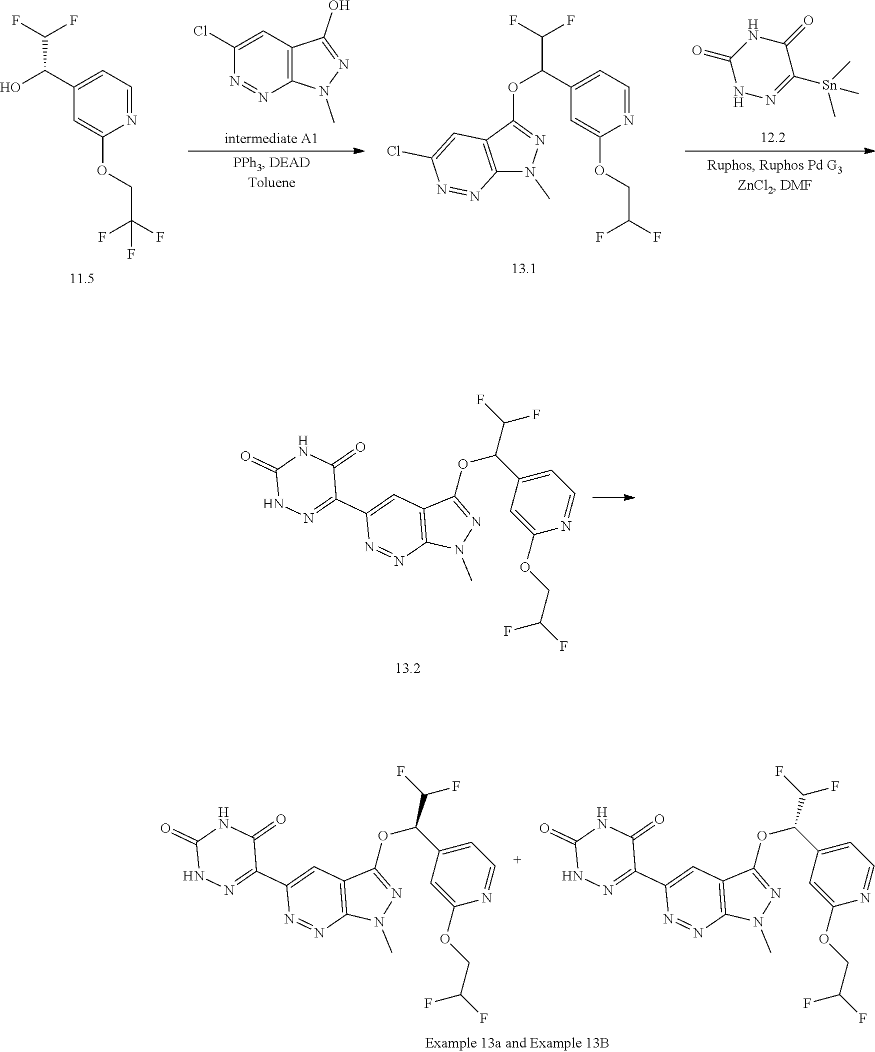

### Step (a): Preparation of 5-chloro-3-[1-[2-(2,2-difluoroethoxy)-4-pyridyl]-2,2-difluoro-ethoxy]-1-methyl-pyrazolo[3,4-c]pyridazine (compound 13.1)

To a solution of 1-[2-(2,2-difluoroethoxy)-4-pyridyl]-2,2-difluoro-ethanol (compound 11.5, 615.5 mg, 2.6 mmol) and 5-chloro-1-methyl-pyrazolo[3,4-c]pyridazin-3-ol (intermediate A1, 250 mg, 1.4 mmol), triphenylphosphine (426.3 mg, 1.6 mmol) in toluene (5 mL) was added DEAD (283.1 mg, 255.9 μL, 1.6 mmol) dropwise at 60° C. After being stirred at 60° C. for 0.5 h under nitrogen, the reaction mixture was concentrated, and the residue was purified by silica gel chromatography (eluted with PE/EA from 0/1 to 5/1) to afford compound 13.1 (220 mg). calc'd 406.1 [(M+H)⁺], measured 405.9 [(M+H)⁺].

### Step (b): Preparation of 6-[3-[1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione (compound 13.2)

To a degassed vial were added 5-chloro-3-[1-[2-(2,2-difluoroethoxy)-4-pyridyl]-2,2-difluoro-ethoxy]-1-methyl-pyrazolo[3,4-c]pyridazine (compound 13.1, 220 mg, 542.2 μmol), 6-trimethylstannyl-2H-1,2,4-triazine-3,5-dione (compound 12.2, 299.2 mg, 1.1 mmol), dicyclohexyl[2-(2,6-diisopropoxyphenyl)phenyl]phosphane (50.6 mg, 108.5 μmol), methanesulfonato(2-dicyclohexylphosphino-2′,6′-di-i-propoxy-1,1′-biphenyl)(2′-amino-1,1′-biphenyl-2-yl)palladium(ii) (90.7 mg, 108.5 μmol) and zinc chloride (110.9 mg, 813.4 μmol) in DMF(2.5 mL). After stirred at 100° C. for 16 h under nitrogen, the mixture was concentrated. The residue was purified by Prep-HPLC to afford compound 13.2 (100 mg). calc'd 483.1 [(M+H)⁺], measured 483.0 [(M+H)⁺].

### Step (c): Preparation of 6-[3-[(1R)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione and 6-[3-[(1S)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione (Example 13A and Example 13B)

Compound 13.2 (100.0 mg) was resolved by SFC to give two single isomers: Example 13A (faster eluting, 30.8 mg) MS: calc'd 483.1 [(M+H)⁺], measured 483.0 [(M+H)⁺]. ¹H NMR (400 MHz, DMSO-d₆) δ=11.79 (br s, 2H), 8.53 (s, 1H), 8.24 (d, J=5.19 Hz, 1H), 7.29 (dd, J=5.34, 1.07 Hz, 1H), 7.15 (s, 1H), 6.64 (td, J=53.56, 2.75 Hz, 1H), 6.38 (tt, J=54.6, 3.7 Hz, 1H), 6.29-6.37 (m, 1H), 4.51-4.61 (m, 2H), 4.07 (s, 3H); and Example 13B (slower eluting, 33.2 mg) calc'd 483.1 [(M+H)⁺], measured 483.0 [(M+H)⁺]. ¹H NMR (400 MHz, DMSO-d₆) δ=12.18 (br s, 2H), 8.53 (s, 1H), 8.24 (d, J=5.19 Hz, 1H), 7.29 (d, J=5.34 Hz, 1H), 7.15 (s, 1H), 6.64 (td, J=53.71, 2.75 Hz, 1H), 6.38 (tt, J=54.9, 3.5 Hz, 1H), 6.29-6.36 (m, 1H), 4.47-4.65 (m, 2H), 4.07 (s, 3H). SFC condition: Column: (S,S) whelk-ol, 250×30 mm I.D., 5 μm, mobile phase: A for CO₂ and B for Methanol (0.1% NH₃H₂O), gradient: B 30%, flow rate: 80 mL/min, back pressure: 100 bar, Column temperature: 35° C.

### Example 14A and 14B

### 5-[1-methyl-3-[(1S)-1-[6-(2,2,2-trifluoroethoxy)pyrimidin-4-yl]ethoxy]pyrazolo[3,4-c]pyridazin-5-yl]-1H-pyrimidine-2,4-dione and 5-[1-methyl-3-[(1R)-1-[6-(2,2,2-trifluoroethoxy)pyrimidin-4-yl]ethoxy]pyrazolo[3,4-c]pyridazin-5-yl]-1H-pyrimidine-2,4-dione

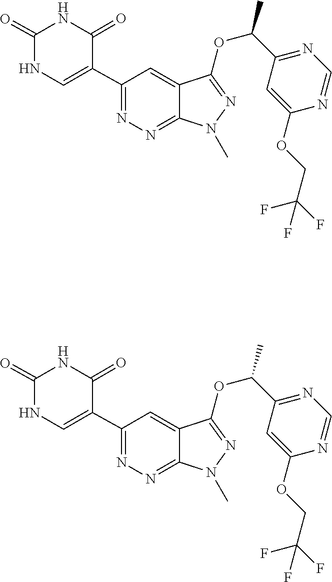 [SMILES: C[C@H](Oc1nn(C)c2nnc(-c3c[nH]c(=O)[nH]c3=O)cc12)c1cc(OCC(F)(F)F)ncn1 ; C[C@@H](Oc1nn(C)c2nnc(-c3c[nH]c(=O)[nH]c3=O)cc12)c1cc(OCC(F)(F)F)ncn1]

The titled compound was synthesized according to the following scheme:

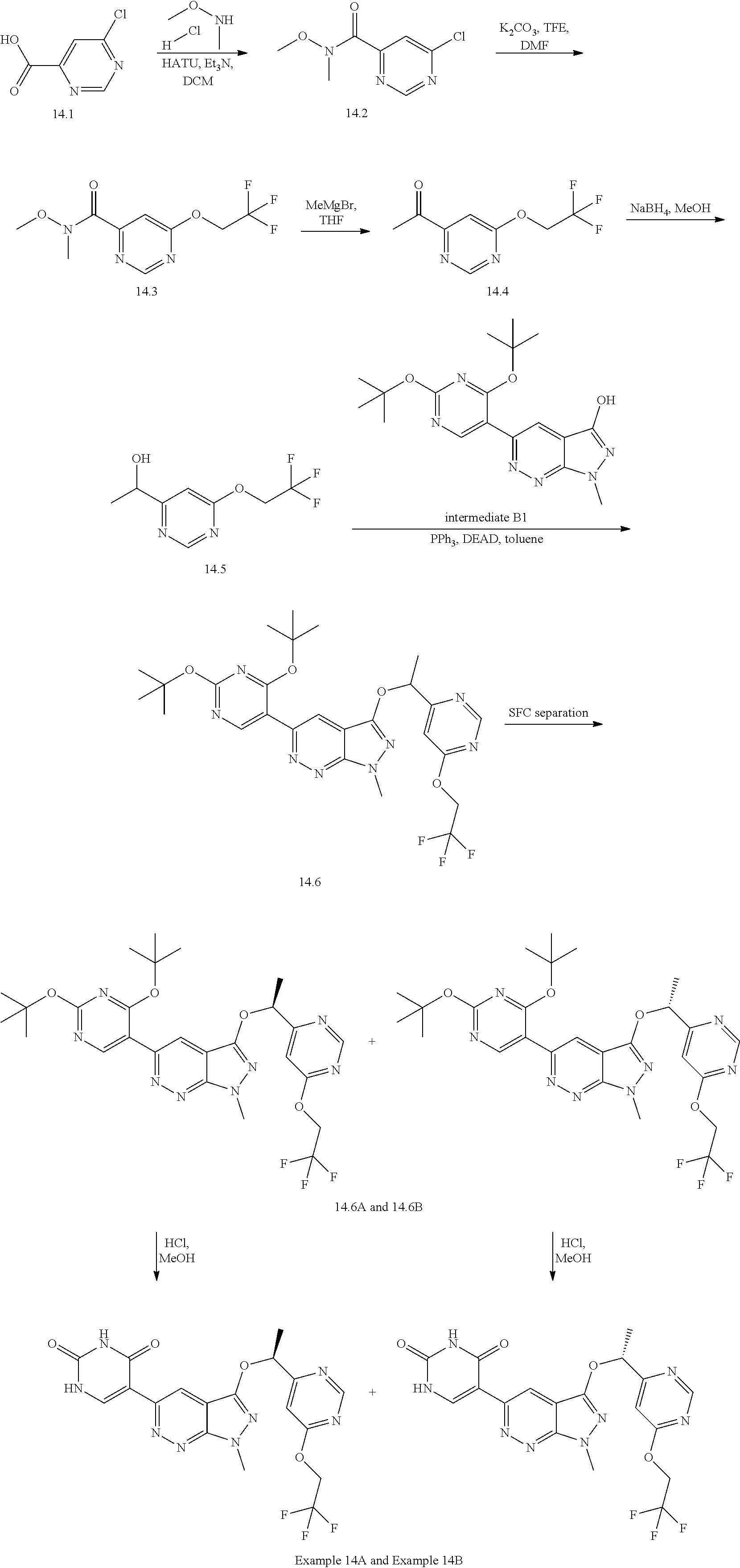

### Step (a): Preparation of 6-chloro-N-methoxy-N-methyl-pyrimidine-4-carboxamide (Compound 14.2)

To a solution of 6-chloro-4-pyrimidinecarboxylic acid (compound 14.1, 3.0 g, 18.9 mmol) in DCM (60 mL) was added N,O-dimethylhydroxylamine hydrochloride (2.0 g, 20.8 mmol), HATU (7.9 g, 20.8 mmol) and Et₃N (3.6 g, 5.0 mL, 35.9 mmol). The resultant mixture was stirred at room temperature under nitrogen for 2 hr. The reaction mixture was diluted with saturated aqueous NaHCO₃ (200 mL), and extracted with DCM (200 mL) for three times. The combined organic layer was washed with brine, dried over Na₂SO₄, filtered and concentrated in vacuo. The residue was purified by flash chromatography (silica gel, 120 g, 0% to 50% EA in PE) to afford compound 14.2 (2.4 g). MS: calc'd 202.0 [(M+H)⁺]; measured 202.0 [(M+H)⁺].

### Step (b): Preparation of N-methoxy-N-methyl-6-(2,2,2-trifluoroethoxy)pyrimidine-4-carboxamide (compound 14.3)

To a solution of 6-chloro-N-methoxy-N-methyl-pyrimidine-4-carboxamide (compound 14.2, 1.7 g, 8.5 mmol) in DMF (15 mL) was added TFE (2.8 g, 2.0 mL, 27.8 mmol) and K₂CO₃ (2.3 g, 17.0 mmol). The resultant mixture was stirred at 50° C. for 2 hr. After being cooled to room temperature, the reaction mixture was diluted with H₂O (100 mL), and extracted with EA (50 mL) for three times. The combined organic layer was washed with brine, dried over Na₂SO₄, filtered and concentrated in vacuo. The residue was purified by flash chromatography (silica gel, 45 g, 0% to 30% EA in PE) to afford compound 14.3 (1.9 g). MS: calc'd 266.1 [(M+H)⁺]; measured 265.9 [(M+H)⁺].

### Step (c): Preparation of 1-[6-(2,2,2-trifluoroethoxy)pyrimidin-4-yl]ethanone (Compound 14.4)

To a solution of N-methoxy-N-methyl-6-(2,2,2-trifluoroethoxy)pyrimidine-4-carboxamide (compound 14.3, 971.4 mg, 3.7 mmol) in THF (15 mL) was added MeMgBr (1.0 M in THF, 5.0 mL, 5.0 mmol). The resultant mixture was stirred at −78° C. for 1 hr. The reaction mixture was diluted with saturated aqueous NH₄Cl (20 mL), and extracted with EA (20 mL) for three times. The combined organic layer was washed with brine, dried over Na₂SO₄, filtered and concentrated in vacuo. The residue was purified by flash chromatography (silica gel, 24 g, 0% to 25% EA in PE) to afford compound 14.4 (476.6 mg). MS: calc'd 221.1 [(M+H)⁺]; measured 221.0 [(M+H)⁺].

### Step (d): Preparation of 1-[6-(2,2,2-trifluoroethoxy)pyrimidin-4-yl]ethanol (Compound 14.5)

To a solution of 1-[6-(2,2,2-trifluoroethoxy)pyrimidin-4-yl]ethanone (compound 14.4, 230.0 mg, 1.0 mmol) in MeOH (2.5 mL) was added NaBH₄ (79.1 mg, 2.1 mmol). The resultant mixture was stirred at 0° C. for 30 min, then diluted with saturated aqueous NaHCO₃ (20 mL), and extracted with EA (20 mL) for three times. The combined organic layer was washed with brine, dried over Na₂SO₄, filtered and concentrated in vacuo. The residue was purified by flash chromatography (silica gel, 12 g, 0% to 35% EA in PE) to afford compound 14.5 (225.0 mg). MS: calc'd 223.1 [(M+H)⁺]; measured 223.0 [(M+H)⁺].

### Step (e): Preparation of 5-(2,4-ditert-butoxypyrimidin-5-yl)-1-methyl-3-[1-[6-(2,2,2-trifluoroethoxy)pyrimidin-4-yl]ethoxy]pyrazolo[3,4-c]pyridazine (compound 14.6)

To a solution of 5-(2,4-ditert-butoxypyrimidin-5-yl)-1-methyl-pyrazolo[3,4-c]pyridazin-3-ol (Intermediate B, 500.0 mg, 1.3 mmol) in toluene (10 mL) was added 1-[6-(2,2,2-trifluoroethoxy)pyrimidin-4-yl]ethanol (compound 14.5, 387.8 mg, 1.8 mmol), PPh₃ (493.0 mg, 1.9 mmol) and DEAD (330.0 mg, 300 μL, 1.9 mmol). The resultant mixture was stirred at 60° C. for 1 hr. After being cooled to room temperature, the reaction mixture was diluted with H₂O (50 mL), and extracted with EA (50 mL) for three times. The combined organic layer was washed with brine, dried over Na₂SO₄, filtered and concentrated in vacuo. The residue was purified by flash chromatography (silica gel, 0% to 35% EA in PE) to afford compound 14.6 (700.0 mg). MS: calc'd 577.2 [(M+H)⁺]; measured 577.3 [(M+H)⁺].

### Step (f): Preparation of 5-(2,4-ditert-butoxypyrimidin-5-yl)-1-methyl-3-[(1S)-1-[6-(2,2,2-trifluoroethoxy)pyrimidin-4-yl]ethoxy]pyrazolo[3,4-c]pyridazine and 5-(2,4-ditert-butoxypyrimidin-5-yl)-1-methyl-3-[(1R)-1-[6-(2,2,2-trifluoroethoxy)pyrimidin-4-yl]ethoxy]pyrazolo[3,4-c]pyridazine (compound 14.6A and 14.6B)

Compound 14.6 (700.0 mg) was resolved by SFC to give two single isomers: compound 14.6A (slower eluting, 307.1 mg) MS: calc'd 577.2 [(M+H)⁺]; measured 577.3 [(M+H)⁺]; and compound 14.6B (faster eluting, 258.4 mg) MS: calc'd 577.2 [(M+H)⁺]; measured 577.3 [(M+H)⁺]. SFC conditions: Column: TCI Chiral MB-S 250×30 mm I.D. 5 μm, mobile phase: A for CO₂ and B for i-PrOH (0.1% NH3·H2O), gradient: B 7%, flow rate: 80 mL/min, back pressure: 100 bar, Column temperature: 35° C.

### Step (g): Preparation of 5-[1-methyl-3-[(1S)-1-[6-(2,2,2-trifluoroethoxy)pyrimidin-4-yl]ethoxy]pyrazolo[3,4-c]pyridazin-5-yl]-1H-pyrimidine-2,4-dione and 5-[1-methyl-3-[(1R)-1-[6-(2,2,2-trifluoroethoxy)pyrimidin-4-yl]ethoxy]pyrazolo[3,4-c]pyridazin-5-yl]-1H-pyrimidine-2,4-dione (Example 14A and Example 14B)

To a solution of compound 14.6A (307.1 mg, 532.6 μmol) in methanol (5 mL) was added HCl (2.0 M in MeOH, 600 μL, 1.2 mmol). The mixture was stirred at room temperature for 1 hr, then concentrated to afford a crude product, which was purified by pre-HPLC to afford Example 14A (199.7 mg). MS: calc'd 465.1 [(M+H)⁺]; measured 465.0 [(M+H)⁺]. ¹H NMR (400 MHz, DMSO-d₆) δ=11.53 (d, J=1.4 Hz, 1H), 11.49-11.41 (m, 1H), 8.87 (d, J=0.9 Hz, 1H), 8.68 (s, 1H), 8.34 (d, J=6.1 Hz, 1H), 7.24 (s, 1H), 5.93 (q, J=6.6 Hz, 1H), 5.19-4.96 (m, 2H), 4.00 (s, 3H), 1.72 (d, J=6.6 Hz, 3H).

To a solution of compound 14B (258.4 mg, 448.2 μmol) in methanol (4 mL) was added HCl (2.0 M in MeOH, 500 μL, 1.0 mmol). The mixture was stirred at room temperature for 1 hr, then concentrated to afford a crude product, which was purified by pre-HPLC to afford Example 14B (153.0 mg). MS: calc'd 465.1 [(M+H)⁺]; measured 465.0 [(M+H)⁺]. ¹H NMR (400 MHz, DMSO-d₆) δ=11.52 (d, J=1.5 Hz, 1H), 11.46-11.38 (m, 1H), 8.86 (d, J=1.1 Hz, 1H), 8.68 (s, 1H), 8.34 (d, J=6.3 Hz, 1H), 7.24 (s, 1H), 5.93 (q, J=6.5 Hz, 1H), 5.16-5.00 (m, 2H), 4.00 (s, 3H), 1.71 (d, J=6.6 Hz, 3H).

### Example 15

### 5-[3-[(1R)-2,2-difluoro-1-[4-fluoro-3-(2-methoxyethoxy)phenyl]ethoxy]-1-methyl-pyrazolo[3,4-c]pyridazin-5-yl]-1H-pyrimidine-2,4-dione

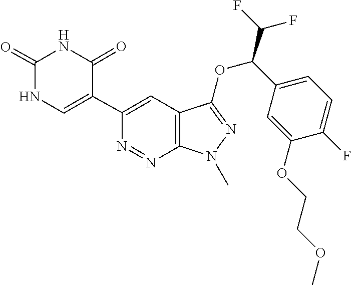 [SMILES: COCCOc1cc([C@@H](Oc2nn(C)c3nnc(-c4c[nH]c(=O)[nH]c4=O)cc23)C(F)F)ccc1F]

The titled compound was synthesized according to the following scheme:

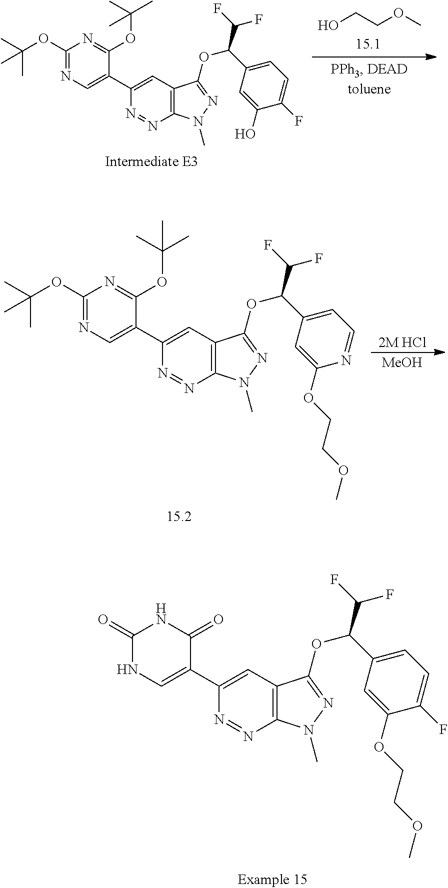

### Step (a): Preparation of 5-(2,4-ditert-butoxypyrimidin-5-yl)-1-methyl-3-[(1R)-2,2-difluoro-1-[4-fluoro-3-(2-methoxyethoxy)phenyl]ethoxy]pyrazolo[3,4-c]pyridazine (Compound 15.2)

To a solution of 5-[(1R)-1-[5-(2,4-ditert-butoxypyrimidin-5-yl)-1-methyl-pyrazolo[3,4-c]pyridazin-3-yl]oxy-2,2-difluoro-ethyl]-2-fluoro-phenol (intermediate E3, 80.0 mg, 146.4 μmol), 2-methoxyethanol (compound 15.1, 16.7 mg, 219.6 μmol) and PPh₃ (57.4 mg, 219.6 μmol) in toluene (0.5 mL) was added DEAD (38.3 mg, 219.6 μmol). The resultant mixture was stirred at 60° C. for 4 hr. After being cooled to room temperature, the reaction mixture concentrated and the residue was purified by flash chromatography (silica gel, 24 g, eluted with 0% to 50% EA in PE) to afford compound 15.2 (69.0 mg). MS: calc'd 493.1[(M+3H-2tBu)⁺], measured 493.1 [(M+3H-2tBu)⁺].

### Step (b): Preparation of 5-[3-[(1R)-2,2-difluoro-1-[4-fluoro-3-(2-methoxyethoxy)phenyl]ethoxy]-1-methyl-pyrazolo[3,4-c]pyridazin-5-yl]-1H-pyrimidine-2,4-dione (Example 15)

To the mixture of 5-(2,4-ditert-butoxypyrimidin-5-yl)-3-[(1R)-2,2-difluoro-1-[4-fluoro-3-(2-methoxyethoxy)phenyl]ethoxy]-1-methyl-pyrazolo[3,4-c]pyridazine (69.0 mg, 114.1 6 μmol) in methanol (0.5 mL) was added a solution of HCl (2.0 M in MeOH, 0.5 mL). The reaction mixture was stirred at room temperature for 1 h, then concentrated to afford a crude product, which was purified by pre-HPLC to afford Example 15 (19.0 mg). MS: calc'd 493.1 [(M+H)⁺]; measured 493.1 [(M+H)⁺]. ¹H NMR (400 MHz, DMSO-d₆) δ=11.54 (d, J=1.5 Hz, 1H), 11.45 (dd, J=1.4, 6.1 Hz, 1H), 8.70 (s, 1H), 8.36 (m, J=6.1 Hz, 1H), 7.43 (dd, J=1.8, 8.3 Hz, 1H), 7.26 (dd, J=8.5, 11.3 Hz, 1H), 7.22-7.14 (m, 1H), 6.60 (dt, J=3.5, 54.3 Hz, 1H), 6.15 (dt, J=3.5, 11.3 Hz, 1H), 4.26-4.12 (m, 2H), 4.03 (s, 3H), 3.66 (t, J=4.5 Hz, 2H), 3.28 (s, 3H).

### Example 16

### 5-[3-[(1R)-1-[2-(2,2-difluoropropoxy)-4-pyridyl]-2,2-difluoro-ethoxy]-1-methyl-pyrazolo[3,4-c]pyridazin-5-yl]-1H-pyrimidine-2,4-dione

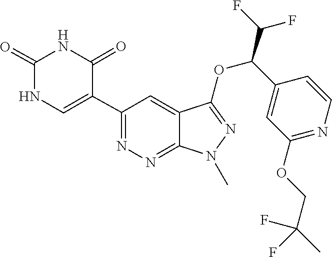 [SMILES: Cn1nc(O[C@H](c2ccnc(OCC(C)(F)F)c2)C(F)F)c2cc(-c3c[nH]c(=O)[nH]c3=O)nnc21]

Example 16 was prepared in analogy to Example 10, by replacing 2,2,2-trifluoroethanol with 2,2-difluoropropan-1-ol in step (b). calc'd 496.1 [(M+H)⁺], measured 496.0 [(M+H)⁺]. ¹H NMR (400 MHz, DMSO-d₆) δ=11.55 (s, 1H), 11.45 (brd, J=6.0 Hz, 1H), 8.72 (s, 1H), 8.37 (d, J=6.3 Hz, 1H), 8.23 (d, J=5.3 Hz, 1H), 7.27 (d, J=5.3 Hz, 1H), 7.15 (s, 1H), 6.63 (td, J=54.0, 2.9 Hz, 1H), 6.35-6.23 (m, 1H), 4.55 (td, J=13.1, 3.7 Hz, 2H), 4.02 (s, 3H), 1.72 (t, J=19.3 Hz, 3H).

### Example 17

### 5-[1-methyl-3-[(1R)-2,2-difluoro-1-[4-(2,2,2-trifluoro-1-methyl-ethoxy)-2-pyridyl]ethoxy]pyrazolo[3,4-c]pyridazin-5-yl]-1H-pyrimidine-2,4-dione

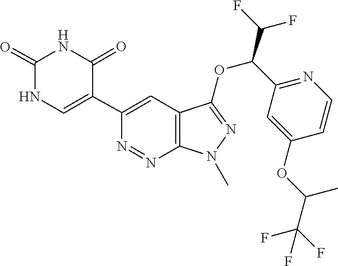 [SMILES: CC(Oc1ccnc([C@@H](Oc2nn(C)c3nnc(-c4c[nH]c(=O)[nH]c4=O)cc23)C(F)F)c1)C(F)(F)F]

Example 17 was prepared in analogy to Example 5, by replacing 1,1-dideuterio-1-deuteriooxy-2,2,2-trifluoro-ethane (compound 5.1) with 1,1,1-trifluoropropan-2-ol in step (a). MS: calc'd 514.1 [(M+H)⁺]; measured 514.1 [(M+H)⁺]. ¹H NMR (400 MHz, DMSO-d₆) δ=11.54 (d, J=1.6 Hz, 1H), 11.45 (dd, J=6.0, 1.4 Hz, 1H), 8.71 (d, J=1.1 Hz, 1H), 8.52 (d, J=5.9 Hz, 1H), 8.37 (d, J=6.1 Hz, 1H), 7.38 (d, J=5.4, 2.5 Hz, 1H), 7.21 (dt, J=5.8, 2.1 Hz, 1H), 6.87-6.52 (m, 1H), 6.24-6.08 (m, 1H), 5.60-5.45 (m, 1H), 4.02 (s, 3H), 1.41 (dd, J=12.6, 6.3 Hz, 3H).

### Example 18

### 5-[1-methyl-3-[(1R)-1-[4-[(2,2-difluorocyclopropyl)methoxy]-2-pyridyl]-2,2-difluoro-ethoxy]pyrazolo[3,4-c]pyridazin-5-yl]-1H-pyrimidine-2,4-dione

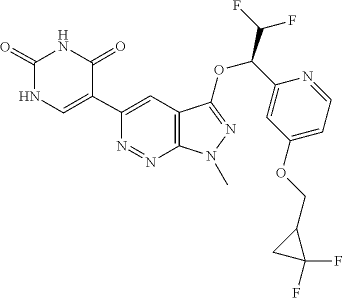 [SMILES: Cn1nc(O[C@H](c2cc(OCC3CC3(F)F)ccn2)C(F)F)c2cc(-c3c[nH]c(=O)[nH]c3=O)nnc21]

The titled compound was synthesized according to the following scheme:

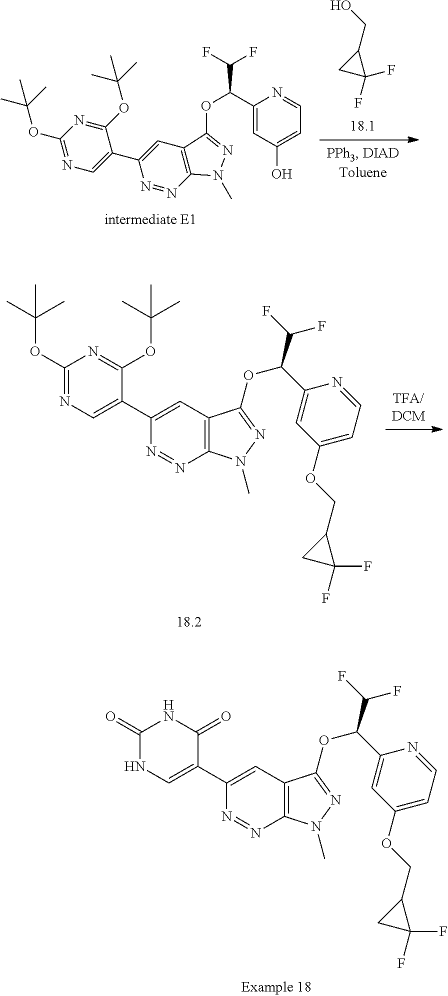 [SMILES: Cn1nc(O[C@H](c2cc(O)ccn2)C(F)F)c2cc(-c3cnc(OC(C)(C)C)nc3OC(C)(C)C)nnc21 ; OCC1CC1(F)F ; Cn1nc(O[C@H](c2cc(OCC3CC3(F)F)ccn2)C(F)F)c2cc(-c3cnc(OC(C)(C)C)nc3OC(C)(C)C)nnc21 ; Cn1nc(O[C@H](c2cc(OCC3CC3(F)F)ccn2)C(F)F)c2cc(-c3c[nH]c(=O)[nH]c3=O)nnc21]

### Step (a): Preparation of 5-(2,4-ditert-butoxypyrimidin-5-yl)-3-[(1R)-1-[4-[(2,2-difluorocyclopropyl)methoxy]-2-pyridyl]-2,2-difluoro-ethoxy]-1-methyl-pyrazolo[3,4-c]pyridazine (compound 18.2)

To a solution of 2-[(1R)-1-[5-(2,4-ditert-butoxypyrimidin-5-yl)-1-methyl-pyrazolo[3,4-c]pyridazin-3-yl]oxy-2,2-difluoro-ethyl]pyridin-4-ol (intermediate E1, 150 mg, 283.4 μmol) in toluene (5 mL) was added 2,2-difluorocyclopropylmethanol (compound 18.1, 61.2 mg, 566.5 μmol) and triphenylphosphine (149 mg, 566.5 μmol) at room temperature. Then diisopropyl azodicarboxylate (115 mg, 566.5 μmol) was added dropwise at room temperature. The resultant mixture was stirred at 100° C. for 2 h. After being cooled to room temperature, the reaction mixture was diluted with water (50 mL), and extracted with EA (30 mL) for three times. The combined organic layer was concentrated. The residue was purified by silica gel column chromatography (eluted with 0 to 70% EA in PE) to afford compound 18.2 (120 mg). MS: calc'd 620.3 [(M+H)⁺]; measured 620.3 [(M+H)⁺].

### Step (b): Preparation of 5-[1-methyl-3-[(1R)-1-[4-[(2,2-difluorocyclopropyl)methoxy]-2-pyridyl]-2,2-difluoro-ethoxy]pyrazolo[3,4-c]pyridazin-5-yl]-1H-pyrimidine-2,4-dione (Example 18)

To a solution of 5-(2,4-ditert-butoxypyrimidin-5-yl)-3-[(1R)-1-[4-[(2,2-difluorocyclopropyl)methoxy]-2-pyridyl]-2,2-difluoro-ethoxy]-1-methyl-pyrazolo[3,4-c]pyridazine (compound 18.2, 115 mg, 185.7 μmol) in DCM (10 mL) was added TFA (0.5 mL). The reaction mixture was stirred at room temperature for 1 h, then concentrated to afford a crude product, which was purified by pre-HPLC to afford Example 18 (47.1 mg). MS: calc'd 508.1 [(M+H)⁺]; measured 508.2 [(M+H)⁺]. ¹H NMR (400 MHz, DMSO-d₆) δ=11.40 (brs, 2H), 8.71 (s, 1H), 8.46 (d, J=5.5 Hz, 1H), 8.38 (s, 1H), 7.25 (s, 1H), 7.05 (dd, J=5.4, 1.9 Hz, 1H), 6.69 (td, J=54.3, 2.3 Hz, 1H), 6.24-6.05 (m, 1H), 4.33-4.20 (m, 1H), 4.16-4.05 (m, 1H), 4.03 (s, 3H), 2.31-2.14 (m, 1H), 1.80-1.65 (m, 1H), 1.59-1.43 (m, 1H).

### Example 19

### 5-[3-[(1R)-2,2-difluoro-1-[4-[(1-fluorocyclopropyl)methoxy]-2-pyridyl]ethoxy]-1-methyl-pyrazolo[3,4-c]pyridazin-5-yl]-1H-pyrimidine-2,4-dione

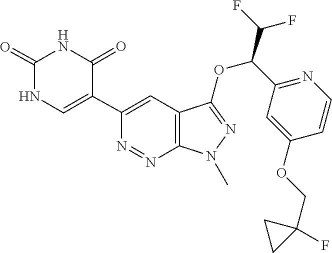 [SMILES: Cn1nc(O[C@H](c2cc(OCC3(F)CC3)ccn2)C(F)F)c2cc(-c3c[nH]c(=O)[nH]c3=O)nnc21]

Example 19 was prepared in analogy to Example 18, by replacing 2,2-difluorocyclopropylmethanol (compound 18.1) with (1-fluorocyclopropyl)methanol in step (a). MS: calc'd 490.1 [(M+H)⁺]; measured 490.3 [(M+H)⁺]. ¹H NMR (400 MHz, DMSO-d₆) δ=11.49 (br, 2H), 8.71 (s, 1H), 8.46 (d, J=5.8 Hz, 1H), 8.37 (s, 1H), 7.26 (d, J=2.5 Hz, 1H), 7.06 (dd, J=5.8, 2.5 Hz, 1H), 6.69 (td, J=54.5, 3.8 Hz, 1H), 6.21-6.09 (m, 1H), 4.41 (d, J=22.8 Hz, 2H), 4.03 (s, 3H), 1.17-1.04 (m 2H), 0.95-0.77 (m, 2H).

### Example 20

### 5-[3-[(1R)-1-(4-ethoxy-2-pyridyl)-2,2-difluoro-ethoxy]-1-methyl-pyrazolo[3,4-c]pyridazin-5-yl]-1H-pyrimidine-2,4-dione

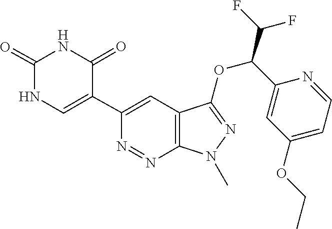 [SMILES: CCOc1ccnc([C@@H](Oc2nn(C)c3nnc(-c4c[nH]c(=O)[nH]c4=O)cc23)C(F)F)c1]

Example 20 was prepared in analogy to Example 4, by replacing 2,2,2-trifluoroethyl trifluoromethanesulfonate with iodoethane in step (a). MS: calc'd 446.1 [(M+H)⁺]; measured 446.0 [(M+H)⁺]. ¹H NMR (400 MHz, DMSO-d₆) δ 11.47 (s, 2H), 8.71 (s, 1H), 8.43 (brd, J=5.5 Hz, 1H), 8.38 (s, 1H), 7.18 (s, 1H), 7.03-6.94 (m, 1H), 6.72 (t, J=54.0 Hz, 1H), 6.22-6.06 (m, 1H), 4.12 (q, J=6.4 Hz, 2H), 4.03 (s, 3H), 1.31 (t, J=6.9 Hz, 3H).

### Example 21

### 2-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxy]acetonitrile

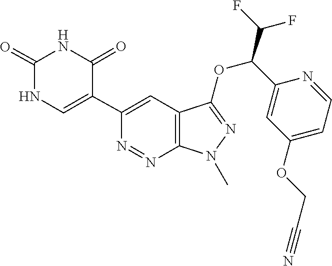 [SMILES: Cn1nc(O[C@H](c2cc(OCC#N)ccn2)C(F)F)c2cc(-c3c[nH]c(=O)[nH]c3=O)nnc21]

Example 21 was prepared in analogy to Example 4, by replacing 2,2,2-trifluoroethyl trifluoromethanesulfonate with 2-bromoacetonitrile in step (a). MS: calc'd 457.1 [(M+H)⁺]; measured 457.2 [(M+H)⁺]. ¹H NMR (400 MHz, DMSO-d₆) δ 11.55 (s, 1H), 11.45 (brd, J=5.8 Hz, 1H), 8.71 (s, 1H), 8.57 (d, J=5.8 Hz, 1H), 8.38 (d, J=6.0 Hz, 1H), 7.35 (s, 1H), 7.20-7.12 (m, 1H), 6.71 (td, J=53.8, 2.5 Hz, 1H), 6.27-6.12 (m, 1H), 5.29 (s, 2H), 4.02 (s, 3H).

### Example 22

### 1-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxymethyl]cyclopropanecarbonitrile

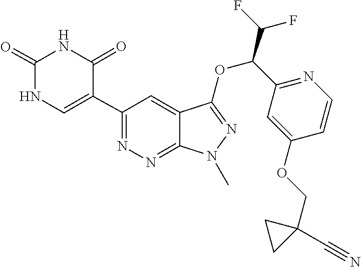 [SMILES: Cn1nc(O[C@H](c2cc(OCC3(C#N)CC3)ccn2)C(F)F)c2cc(-c3c[nH]c(=O)[nH]c3=O)nnc21]

Example 22 was prepared in analogy to Example 4, by replacing 2,2,2-trifluoroethyl trifluoromethanesulfonate with 1-(bromomethyl) cyclopropanecarbonitrile in step (a). MS: calc'd 497.1 [(M+H)⁺]; measured 497.2 [(M+H)⁺]. ¹H NMR (400 MHz, DMSO-d₆) δ=11.54 (s, 1H), 11.49 (brs, 1H), 8.71 (s, 1H), 8.47 (d, J=5.8 Hz, 1H), 8.38 (s, 1H), 7.24 (d, J=2.3 Hz, 1H), 7.03 (dd, J=5.8, 2.5 Hz, 1H), 6.69 (td, J=53.8, 3.5 Hz, 1H), 6.15 (ddd, J=14.3, 7.8, 3.6 Hz, 1H), 4.15 (s, 2H), 4.03 (s, 3H), 1.37 (dd, J=7.5, 5.0 Hz, 2H), 1.17 (d, J=7.0, 4.5 Hz, 2H).

### Example 23

### 5-[3-[(1R)-2,2-difluoro-1-[4-[[1-(trifluoromethyl)cyclopropyl]methoxy]-2-pyridyl]ethoxy]-1-methyl-pyrazolo[3,4-c]pyridazin-5-yl]-1H-pyrimidine-2,4-dione

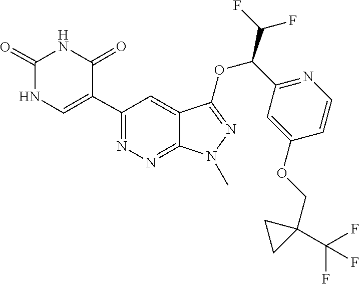 [SMILES: Cn1nc(O[C@H](c2cc(OCC3(C(F)(F)F)CC3)ccn2)C(F)F)c2cc(-c3c[nH]c(=O)[nH]c3=O)nnc21]

Example 23 was prepared in analogy to Example 4, by replacing 2,2,2-trifluoroethyl trifluoromethanesulfonate with 1-(bromomethyl)-1-(trifluoromethyl) cyclopropane in step (a). MS: calc'd 540.1 [(M+H)⁺]; measured 540.2 [(M+H)⁺]. ¹H NMR (400 MHz, DMSO-d₆) δ=11.51 (brs, 2H), 8.71 (s, 1H), 8.45 (d, J=5.8 Hz, 1H), 8.38 (s, 1H), 7.23 (d, J=2.3 Hz, 1H), 7.03 (dd, J=5.8, 2.3 Hz, 1H), 6.68 (td, J=54.0, 3.8 Hz, 1H), 6.15 (ddd, J=14.3, 7.8, 3.4 Hz, 1H), 4.23 (s, 2H), 4.03 (s, 3H), 1.13-1.05 (m, 2H), 1.05-0.97 (m, 2H).

### Example 24

### 5-[3-[(1R)-2,2-difluoro-1-[1-(2,2,2-trifluoroethyl)indazol-6-yl]ethoxy]-1-methyl-pyrazolo[3,4-c]pyridazin-5-yl]-1H-pyrimidine-2,4-dione

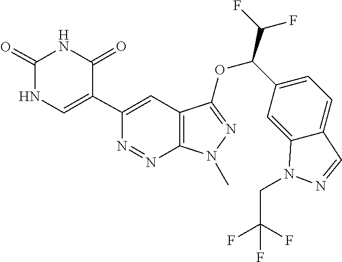 [SMILES: Cn1nc(O[C@H](c2ccc3cnn(CC(F)(F)F)c3c2)C(F)F)c2cc(-c3c[nH]c(=O)[nH]c3=O)nnc21]

The titled compound was synthesized according to the following scheme:

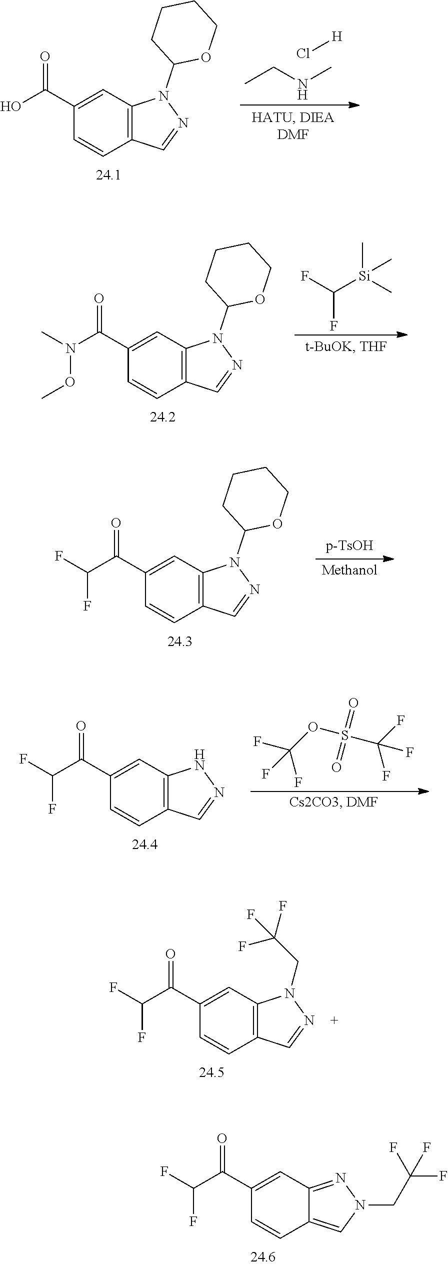

### Step (a): Preparation of N-methoxy-N-methyl-1-tetrahydropyran-2-yl-indazole-6-carboxamide (compound 24.2)

To a solution of 1-tetrahydropyran-2-ylindazole-6-carboxylic acid (compound 24.1, 5.2 g, 21.12 mmol) in DMF (45 mL) were added HATU (8.43 g, 22.17 mmol), N,O-dimethylhydroxylamine hydrochloride (2.47 g, 25.34 mmol) and DIPEA (13.65 g, 18.39 mL, 105.58 mmol). The resultant mixture was stirred at 20° C. for 1 hr, then diluted with water (300 mL), extracted with EA (50 mL) for three times. The combined organic layer was washed with brine, dried over Na₂SO₄, filtered and concentrated. The residue was purified by flash chromatography (silica gel, 120 g, eluted with 15% to 90% EA in PE) to afford compound 24.2 (5.2 g). MS: calc'd 290.1 [(M+H)⁺]; measured 290.0 [(M+H)⁺].

### Step (b): Preparation of 2,2-difluoro-1-(1-tetrahydropyran-2-yl-indazol-6-yl)ethanone (Compound 24.3)

To a solution of N-methoxy-N-methyl-1-tetrahydropyran-2-yl-indazole-6-carboxamide (compound 24.2, 1.0 g, 3.46 mmol) and difluoromethyltrimethysilane (858.54 mg, 6.91 mmol) in THF (15 mL) was added potassium tert-butoxide (698.09 mg, 6.22 mmol) at 0° C. The resultant mixture was stirred at 0° C. for 16 hr. The reaction was quenched with saturated aqueous NH₄Cl (10 mL), extracted with EA (20 mL) for three times. The combined organic layer was washed with brine, dried over Na₂SO₄, filtered and concentrated. The residue was purified by flash chromatography (silica gel, eluted with 0% to 25% EA in PE) to afford compound 24.3 (300 mg).

### Step (c): Preparation of 2,2-difluoro-1-(1H-indazol-6-yl)ethanone (Compound 24.4)

To a solution of 2,2-difluoro-1-(1-tetrahydropyran-2-ylindazol-6-yl)ethanone (compound 24.3, 300 mg, 1.07 mmol) in methanol (3 mL) was added p-toluenesulfonic acid (204 mg, 1.07 mmol). The resultant mixture was stirred at 50° C. for 16 h. The reaction mixture was concentrated, and the residue was purified by flash chromatography (silica gel, eluted with 0% to 60% EA in DCM) to afford compound 24.4 (200 mg). MS: calc'd 197.0 [(M+H)⁺]; measured 215.0 [(M+H₂O+H)⁺].

### Step (d): Preparation of 2,2-difluoro-1-[1-(2,2,2-trifluoroethyl)indazol-6-yl]ethanone (Compound 24.5)

To a solution of 2,2-difluoro-1-(1H-indazol-6-yl)ethanone (compound 24.4, 200 mg, 1.02 mmol) in DMF (3 mL) was added cesium carbonate (996.65 mg, 3.06 mmol) and 2,2,2-trifluoroethyl trifluoromethanesulfonate (283.99 mg, 1.22 mmol). The resultant mixture was stirred at 20° C. for 2 hr. The reaction was quenched with H₂O (50 mL), and extracted with EA (10 mL) for three times. The combined organic layer was washed with brine, dried over Na₂SO₄, filtered and concentrated. The residue was purified by flash chromatography (silica gel, 12 g, eluted with 0% to 15% EA in PE) to afford compound 24.5 (100 mg) and compound 24.6 (90 mg).

### Step (e): Preparation of (1S)-2,2-difluoro-1-[1-(2,2,2-trifluoroethyl)indazol-6-yl]ethanol (compound 24.7)

To a solution of 2,2-difluoro-1-[1-(2,2,2-trifluoroethyl)indazol-6-yl]ethanone (compound 24.5, 100 mg, 359.48 μmol) in dichloromethane (10 mL) were added formic acid (248.2 mg, 5.39 mmol) and triethylamine (218.25 mg, 2.16 mmol). And then RuCl(p-cymene)[(R,R)-Ts-DPEN](22.87 mg, 35.95 μmol) was added. The resultant mixture was stirred at 20° C. for 1 h. The reaction was quenched with saturated aqueous NaHCO₃ (2 mL), diluted with water (20 mL), and extracted with DCM (5 mL) for three times. The combined organic layer was washed with brine, dried over Na₂SO₄, filtered and concentrated. The residue was purified by flash chromatography (silica gel, 12 g, eluted with 0% to 25% EA in PE) to afford compound 24.7 (86 mg). MS: calc'd 281.1 [(M+H)⁺]; measured 281.1 [(M+H)⁺].

### Step (f): Preparation of 5-(2,4-ditert-butoxypyrimidin-5-yl)-3-[(1R)-2,2-difluoro-1-[1-(2,2,2-trifluoroethyl)indazol-6-yl]ethoxy]-1-methyl-pyrazolo[3,4-c]pyridazine (compound 24.8)

To the suspension of (1S)-2,2-difluoro-1-[1-(2,2,2-trifluoroethyl)indazol-6-yl]ethanol (compound 24.7, 86 mg, 306.93 μmol), 5-(2,4-ditert-butoxypyrimidin-5-yl)-1-methyl-pyrazolo[3,4-c]pyridazin-3-ol (intermediate B1, 110 mg, 295.37 μmol), and triphenylphosphine (92.97 mg, 354.44 μmol) in toluene (2 mL) was added DIAD (61.73 mg, 354.44 μmol). The resultant mixture was stirred at 50° C. for 0.5 h under nitrogen. After being cooled to room temperature, the reaction mixture was diluted with water (50 mL), extracted with EA (30 mL) for three times. The combined organic layer was washed with brine, dried over Na₂SO₄, filtered and concentrated. The residue was purified by flash chromatography (silica gel, 40 g, eluted with 0% to 25% EA in PE) to afford compound 24.8 (100 mg). MS: calc'd 634.2 [(M+H)⁺]; measured 523.1 [(M−112+H)⁺].

### Step (g): Preparation of 5-[3-[(1R)-2,2-difluoro-1-[1-(2,2,2-trifluoroethyl)indazol-6-yl]ethoxy]-1-methyl-pyrazolo[3,4-c]pyridazin-5-yl]-1H-pyrimidine-2,4-dione (Example 24)

To a solution of 5-(2,4-ditert-butoxypyrimidin-5-yl)-3-[(1R)-2,2-difluoro-1-[1-(2,2,2-trifluoroethyl)indazol-6-yl]ethoxy]-1-methyl-pyrazolo[3,4-c]pyridazine (compound 24.8, 100 mg, 157.58 μmol) in methanol (1 mL) was added 2 M HCl (236.37 μL, 472.74 μmol). The reaction mixture was stirred at room temperature for 1 h, then concentrated to afford a crude product, which was purified by pre-HPLC to afford Example 24 (24.8 mg). MS: calc'd 523.1 [(M+H)⁺]; measured 523.1 [(M+H)⁺]. ¹H NMR (400 MHz, DMSO-d₆) δ=11.55 (d, J=1.8 Hz, 1H), 11.48-11.37 (m, 1H), 8.74 (s, 1H), 8.36 (d, J=6.3 Hz, 1H), 8.23 (d, J=0.9 Hz, 1H), 8.08 (s, 1H), 7.86 (d, J=8.3 Hz, 1H), 7.49-7.39 (m, 1H), 6.65 (dt, J=3.9, 54.2 Hz, 1H), 6.31 (dt, J=3.3, 11.3 Hz, 1H), 5.52-5.41 (m, 2H), 4.00 (s, 3H).

### Example 25

### 5-[3-[(1R)-2,2-difluoro-1-[4-(2,2,2-trifluoroethyl)-2,3-dihydro-1,4-benzoxazin-6-yl]ethoxy]-1-methyl-pyrazolo[3,4-c]pyridazin-5-yl]-1H-pyrimidine-2,4-dione

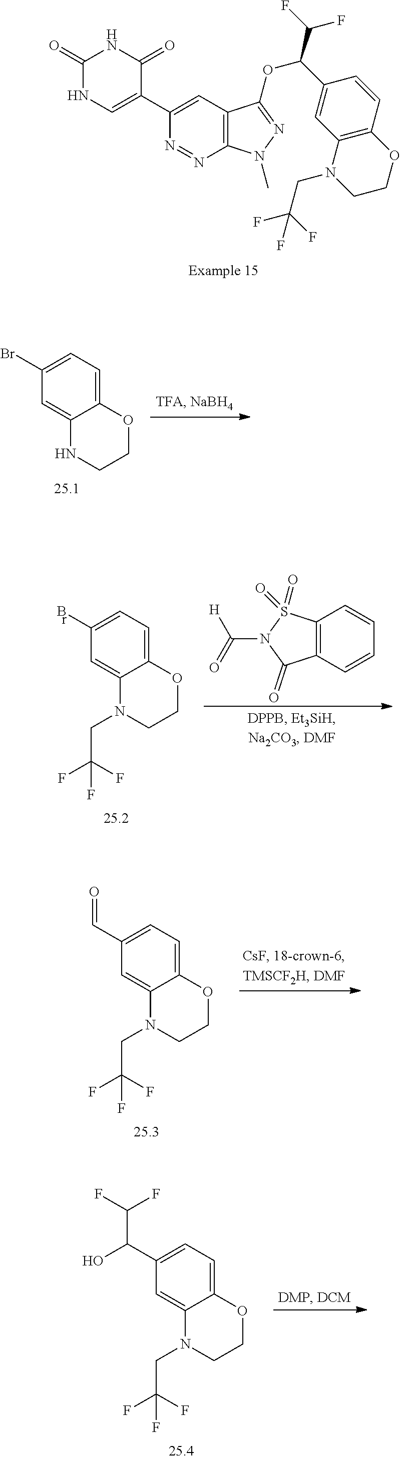

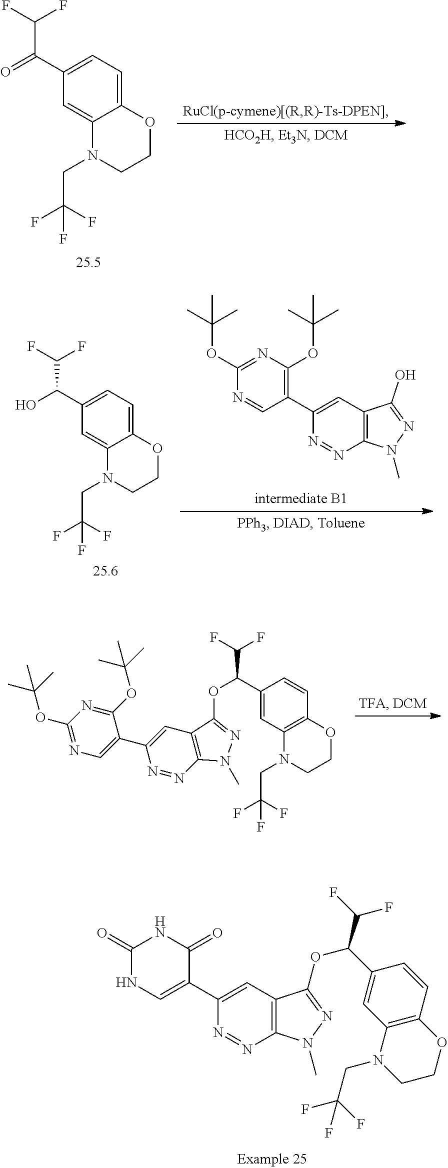 [SMILES: O=C(c1ccc2c(c1)N(CC(F)(F)F)CCO2)C(F)F ; O[C@@H](c1ccc2c(c1)N(CC(F)(F)F)CCO2)C(F)F ; Cn1nc(O)c2cc(-c3cnc(OC(C)(C)C)nc3OC(C)(C)C)nnc21 ; Cn1nc(O[C@H](c2ccc3c(c2)N(CC(F)(F)F)CCO3)C(F)F)c2cc(-c3cnc(OC(C)(C)C)nc3OC(C)(C)C)nnc21 ; Cn1nc(O[C@H](c2ccc3c(c2)N(CC(F)(F)F)CCO3)C(F)F)c2cc(-c3c[nH]c(=O)[nH]c3=O)nnc21]

### Step (a): Preparation of 6-bromo-4-(2,2,2-trifluoroethyl)-2,3-dihydro-1,4-benzoxazine (Compound 25.2)

A solution of 6-bromo-3,4-dihydro-2H-1,4-benzoxazine (compound 25.1, 3.00 g, 8.4 mmol) in TFA (30 mL) was stirred at room temperature for 1 h under nitrogen atmosphere, followed by the addition of NaBH₄ (1.27 g, 33.7 mmol) in portions at 0° C. The resultant mixture was stirred at 70° C. for overnight, then concentrated, the residue was diluted with EA (300 mL), and washed with brine (100 mL) twice. The organic layer was dried over Na₂SO₄, filtered and concentrated. The residue was purified by flash chromatography (silica gel, eluted with 0% to 100% EA in PE) to afford compound 25.2 (1.2 g). ¹H NMR (400 MHz, DMSO-d₆) δ 7.05 (d, J=2.2 Hz, 1H), 6.74 (dd, J=8.4, 2.2 Hz, 1H), 6.67 (d, J=8.4 Hz, 1H), 4.24 (q, J=9.7 Hz, 2H), 4.15-4.10 (m, 2H), 3.46 (t, J=4.4 Hz, 2H).

### Step (b): Preparation of 4-(2,2,2-trifluoroethyl)-2,3-dihydro-1,4-benzoxazine-6-carbaldehyde (compound 25.3)

To a stirred mixture of 6-bromo-4-(2,2,2-trifluoroethyl)-2,3-dihydro-1,4-benzoxazine (compound 25.2, 1.2 g, 4.05 mmol), palladium (II) acetate (45.5 mg, 0.20 mmol), 1,4-bis(diphenylphosphino)butane (173 mg, 0.41 mmol) and sodium carbonate (1.07 g, 10.1 mmol) in DMF (6 mL) were added 1,1,3-trioxo-1,2-benzothiazole-2-carbaldehyde (1.28 g, 6.08 mmol) and triethylsilane (943 mg, 8.11 mmol) in portions at room temperature under nitrogen atmosphere. The resultant mixture was stirred at 80° C. for overnight under nitrogen atmosphere. After being cooled to room temperature, the reaction was quenched with water (50 mL), and extracted with EA (100 mL) for three times. The combined organic layer was dried over anhydrous Na₂SO₄, filtrated and concentrated. The residue was purified by silica gel chromatography (eluted with 0 to 100% EA in PE) to afford compound 25.3 (620 mg). MS: calc'd 246.1 [(M+H)⁺]; measured 246.0 [(M+H)⁺].

### Step (c): Preparation of 2,2-difluoro-1-[4-(2,2,2-trifluoroethyl)-2,3-dihydro-1,4-benzoxazin-6-yl]ethanol (compound 25.4)

To a stirred mixture of 4-(2,2,2-trifluoroethyl)-2,3-dihydro-1,4-benzoxazine-6-carbaldehyde (compound 25.3, 620 mg, 2.53 mmol), cesium fluoride (38.4 mg, 0.25 mmol) and 18-crown-6 (134 mg, 0.51 mmol) in DMF (12 mL) was added (difluoromethyl)trimethylsilane (503 mg, 4.05 mmol) dropwise at −20° C. under nitrogen atmosphere. The resultant mixture was stirred at −20° C. for 3 h under nitrogen atmosphere. The reaction was quenched with water (100 mL), and extracted with EA (150 mL) for three times. The combined organic layer was dried over anhydrous Na₂SO₄, filtrated and concentrated. The residue was purified by silica gel chromatography (eluted with 0 to 100% EA in PE) to afford compound 25.4 (153 mg). MS: calc'd 298.1 [(M+H)⁺]; measured 297.9 [(M+H)⁺].

### Step (d): Preparation of 2,2-difluoro-1-[4-(2,2,2-trifluoroethyl)-2,3-dihydro-1,4-benzoxazin-6-yl]ethanone (compound 25.5)

A mixture of 2,2-difluoro-1-[4-(2,2,2-trifluoroethyl)-2,3-dihydro-1,4-benzoxazin-6-yl]ethanol (compound 25.4, 150 mg, 0.5 mmol), DMP (750 mg, 1.77 mmol) and sodium carbonate (214 mg, 2.02 mmol) in DCM (20 mL) was stirred at room temperature for 3 h under nitrogen atmosphere. The reaction was quenched with water (50 mL), and extracted with DCM (50 mL) twice. The combined organic layer was dried over anhydrous Na₂SO₄, filtrated and concentrated. The residue was purified by silica gel chromatography (eluted with 0 to 100% EA in PE) to afford compound 25.5 (115 mg). MS: calc'd 296.1 [(M+H)⁺]; measured 296.0 [(M+H)⁺].

### Step (e): Preparation of 5-[3-[(1R)-2,2-difluoro-1-[4-(2,2,2-trifluoroethyl)-2,3-dihydro-1,4-benzoxazin-6-yl]ethoxy]-1-methyl-pyrazolo[3,4-c]pyridazin-5-yl]-1H-pyrimidine-2,4-dione (Example 25)

Example 25 was prepared in analogy to Example 24, by replacing 2,2-difluoro-1-[1-(2,2,2-trifluoroethyl)indazol-6-yl]ethanone (compound 24.5) with 2,2-difluoro-1-[4-(2,2,2-trifluoroethyl)-2,3-dihydro-1,4-benzoxazin-6-yl]ethanone (compound 25.5) cyclopropane in step (e). MS: calc'd 540.1 [(M+H)⁺]; measured 540.4 [(M+H)⁺]. ¹H NMR (400 MHz, DMSO-d₆) δ 11.74-11.34 (br, 2H), 8.66 (s, 1H), 8.37 (s, 1H), 7.12 (s, 1H), 6.90-6.72 (m, 2H), 6.54 (td, J=54.6, 4.0 Hz, 1H), 6.00 (dt, J=11.0, 6.8 Hz, 1H), 4.26-4.09 (m, 4H), 4.03 (s, 3H), 3.44 (m, 2H).

### Example 26

### 5-[3-[(1R)-2,2-difluoro-1-[2-[[(1S)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione

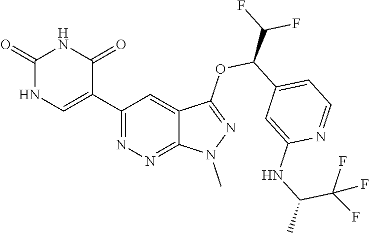 [SMILES: C[C@H](Nc1cc([C@@H](Oc2nn(C)c3nnc(-c4c[nH]c(=O)[nH]c4=O)cc23)C(F)F)ccn1)C(F)(F)F]

The titled compound was synthesized according to the following scheme:

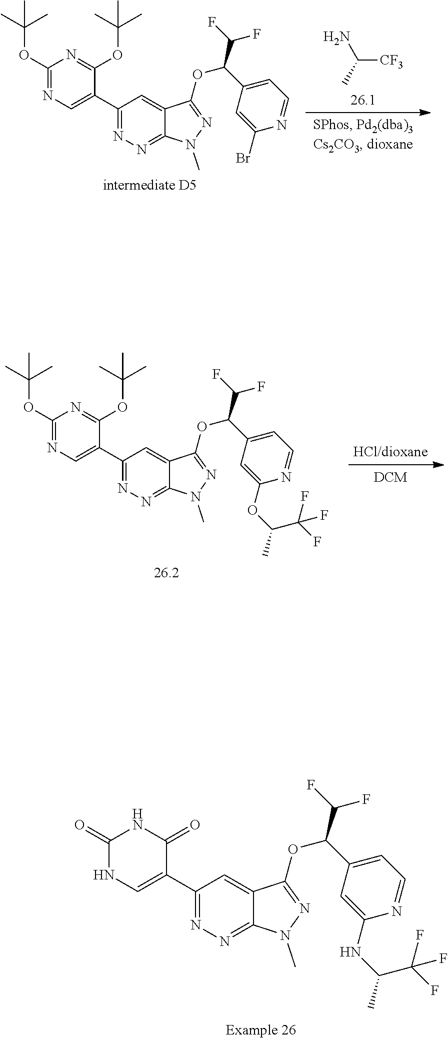

### Step (a): Preparation of 4-[(1R)-1-[5-(2,4-ditert-butoxypyrimidin-5-yl)-1-methyl-pyrazolo[3,4-c]pyridazin-3-yl]oxy-2,2-difluoro-ethyl]-N-[(1S)-2,2,2-trifluoro-1-methyl-ethyl]pyridin-2-amine (Compound 26.2)

To a solution of 3-[(1R)-1-(2-bromo-4-pyridyl)-2,2-difluoro-ethoxy]-5-(2,4-ditert-butoxypyrimidin-5-yl)-1-methyl-pyrazolo[3,4-c]pyridazine (intermediate D5, 200 mg, 0.34 mmol) in 1,4-dioxane (10 mL) was added (2S)-1,1,1-trifluoropropan-2-amine (compound 26.1, 191 mg, 1.69 mmol), Cs₂CO₃ (220 mg, 0.68 mmol), Pd₂(dba)₃ (61.8 mg, 0.07 mmol) and SPhos (27.7 mg, 0.07 mmol). The resultant mixture was stirred at 100° C. for 2 h under nitrogen atmosphere. After being cooled to room temperature, the reaction mixture was diluted with water (30 mL), and extracted with EA (20 mL) for three times. The combined organic layer was washed with brine (20 mL), dried over anhydrous Na₂SO₄, filtered and concentrated. The residue was purified by flash chromatography (silica gel, 20 g, 0 to 100% EA in PE) to afford compound 26.6 (145.0 mg). MS: calc'd 625.3 [(M+H)⁺]; measured 625.4 [(M+H)⁺].

### Step (b): Preparation of 5-[3-[(1R)-2,2-difluoro-1-[2-[[(1S)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione (Example 26)

To a solution of 4-[(1R)-1-[5-(2,4-ditert-butoxypyrimidin-5-yl)-1-methyl-pyrazolo[3,4-c]pyridazin-3-yl]oxy-2,2-difluoro-ethyl]-N-[(1S)-2,2,2-trifluoro-1-methyl-ethyl]pyridin-2-amine (compound 26.2, 145.0 mg, 0.28 mmol) in DCM (5 mL) was added 4 N HCl/dioxane (0.5 mL). The reaction mixture was stirred at room temperature for 1 h, then concentrated to afford a crude product, which was purified by pre-HPLC to afford Example 26 (65.1 mg). MS: calc'd 513.1 [(M+H)⁺]; measured 513.4 [(M+H)⁺]. ¹H NMR (400 MHz, DMSO-d₆) δ=11.53 (br s, 2H), 8.72 (s, 1H), 8.40 (s, 1H), 8.04 (d, J=5.3 Hz, 1H), 7.17 (d, J=8.8 Hz, 1H), 6.80 (dd, J=5.3, 1.5 Hz, 1H), 6.77 (s, 1H), 6.56 (td, J=53.8, 2.8 Hz, 1H), 6.19-6.09 (m, 1H), 4.96 (dd, J=15.5, 8.0 Hz, 1H), 4.03 (s, 3H), 1.27 (d, J=7.0 Hz, 3H).

### Example 27

### 5-[3-[(1R)-2,2-difluoro-1-[2-[[(1R)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione

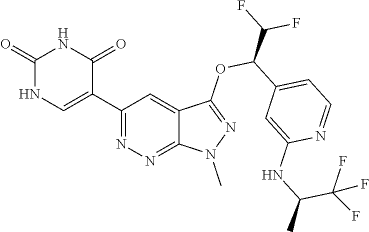 [SMILES: C[C@@H](Nc1cc([C@@H](Oc2nn(C)c3nnc(-c4c[nH]c(=O)[nH]c4=O)cc23)C(F)F)ccn1)C(F)(F)F]

Example 27 was prepared in analogy to Example 26, by replacing (2S)-1,1,1-trifluoropropan-2-amine (compound 26.1) with (2R)-1,1,1-trifluoropropan-2-amine in step (a). MS: calc'd 513.1 [(M+H)⁺]; measured 513.4 [(M+H)⁺]. ¹H NMR (400 MHz, DMSO-d₆) δ=11.52 (s, 2H), 8.72 (s, 1H), 8.40 (s, 1H), 8.04 (d, J=5.3 Hz, 1H), 7.16 (d, J=8.8 Hz, 1H), 6.80 (dd, J=5.3, 1.4 Hz, 1H), 6.76 (s, 1H), 6.56 (td, J=53.9, 3.0 Hz, 1H), 6.13 (td, J=12.0, 3.0 Hz, 1H), 4.96 (m, 1H), 4.03 (s, 3H), 1.25 (d, J=7.0 Hz, 3H).

### Example 28

### 5-[3-[(1R)-2,2-difluoro-1-[2-(2-methoxyethoxy)-4-pyridyl]ethoxy]-1-methyl-pyrazolo[3,4-c]pyridazin-5-yl]-1H-pyrimidine-2,4-dione

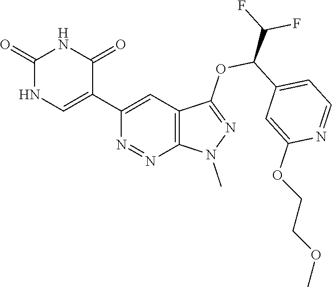 [SMILES: COCCOc1cc([C@@H](Oc2nn(C)c3nnc(-c4c[nH]c(=O)[nH]c4=O)cc23)C(F)F)ccn1]

The titled compound was synthesized according to the following scheme:

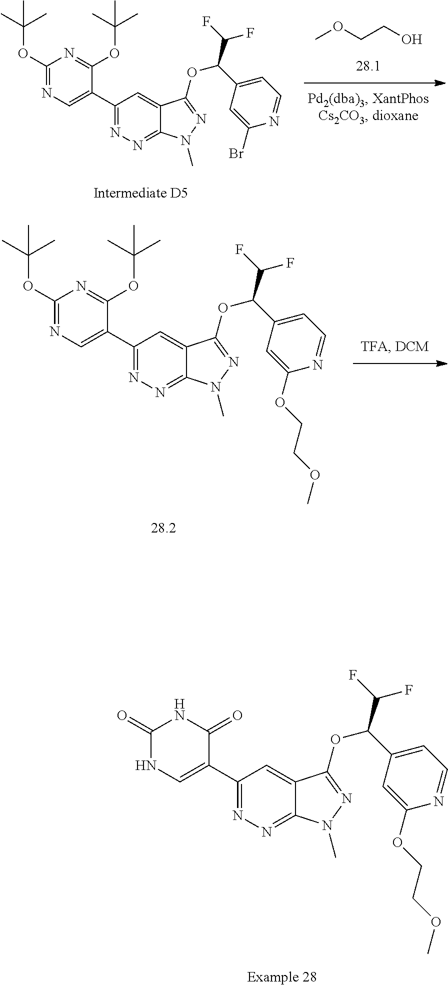

### Step (a): Preparation of 5-(2,4-ditert-butoxypyrimidin-5-yl)-3-[(1R)-2,2-difluoro-1-[2-(2-methoxyethoxy)-4-pyridyl]ethoxy]-1-methyl-pyrazolo[3,4-c]pyridazine (compound 28.2)

To a solution of 3-[(1R)-1-(2-bromo-4-pyridyl)-2,2-difluoro-ethoxy]-5-(2,4-ditert-butoxypyrimidin-5-yl)-1-methyl-pyrazolo[3,4-c]pyridazine (intermediate D5, 100.0 mg, 0.17 mmol) in 1,4-dioxane (10 mL) was added 2-methoxyethanol (compound 28.1, 25.7 mg, 0.34 mmol), Cs₂CO₃ (110 mg, 0.34 mmol), Pd₂(dba)₃ (23.2 mg, 0.03 mmol) and XantPhos (29.3 mg, 0.05 mmol). The resultant mixture was stirred at 100° C. for 2 h under nitrogen atmosphere. After being cooled to room temperature, the reaction mixture was diluted with water (50 mL), and extracted with EA (50 mL) for three times. The combined organic layer was washed with brine (20 mL), dried over anhydrous Na₂SO₄, filtered and concentrated. The residue was purified by flash chromatography (silica gel, 20 g, 0 to 100% EA in PE) to afford compound 28.2 (64.0 mg). MS: calc'd 588.3 [(M+H)⁺]; measured 588.2 [(M+H)⁺].

### Step (e): Preparation of 5-[3-[(1R)-2,2-difluoro-1-[2-(2-methoxyethoxy)-4-pyridyl]ethoxy]-1-methyl-pyrazolo[3,4-c]pyridazin-5-yl]-1H-pyrimidine-2,4-dione (Example 28)

To a solution of 5-(2,4-ditert-butoxypyrimidin-5-yl)-3-[(1R)-2,2-difluoro-1-[2-(2-methoxyethoxy)-4-pyridyl]ethoxy]-1-methyl-pyrazolo[3,4-c]pyridazine (compound 28.2, 62.0 mg, 0.11 mmol) in DCM (5 mL) was added TFA (0.5 mL). The reaction mixture was stirred at room temperature for 1 h, then concentrated to afford a crude product, which was purified by pre-HPLC to afford Example 28 (29.6 mg). MS: calc'd 476.1 [(M+H)⁺]; measured 476.2 [(M+H)⁺]. ¹H NMR (400 MHz, DMSO-d₆) δ 11.34 (br s, 2H), 8.73 (s, 1H), 8.38 (s, 1H), 8.19 (d, J=5.2 Hz, 1H), 7.19 (dd, J=5.2, 1.0 Hz, 1H), 7.03 (s, 1H), 6.62 (td, J=53.8, 3.0 Hz, 1H), 6.29-6.22 (m, 1H), 4.49-4.27 (m, 2H), 4.02 (s, 3H), 3.73-3.54 (m, 2H), 3.28 (s, 3H).

### Example 29

### 5-[3-[(1R)-1-(2-ethoxy-4-pyridyl)-2,2-difluoro-ethoxy]-1-methyl-pyrazolo[3,4-c]pyridazin-5-yl]-1H-pyrimidine-2,4-dione

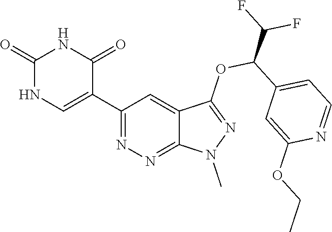 [SMILES: CCOc1cc([C@@H](Oc2nn(C)c3nnc(-c4c[nH]c(=O)[nH]c4=O)cc23)C(F)F)ccn1]

Example 29 was prepared in analogy to Example 28, by replacing 2-methoxyethanol (compound 28.1) with ethanol in step (a). MS: calc'd 446.1 [(M+H)⁺]; measured 446.2 [(M+H)⁺]. ¹H NMR (400 MHz, DMSO-d₆) δ=11.40 (br s, 2H), 8.73 (s, 1H), 8.38 (s, 1H), 8.20 (d, J=5.3 Hz, 1H), 7.18 (dd, J=5.6, 1.4 Hz, 1H), 6.99 (s, 1H), 6.62 (td, J=53.5, 2.5 Hz, 1H), 6.25 (t, J=11.1 Hz, 1H), 4.30 (q, J=7.0 Hz, 2H), 4.03 (s, 3H), 1.30 (t, J=7.0 Hz, 3H).

### Example 30

### 5-[3-[(1R)-2,2-difluoro-1-[2-(1,1,2,2,2-pentadeuterioethoxy)-4-pyridyl]ethoxy]-1-methyl-pyrazolo[3,4-c]pyridazin-5-yl]-1H-pyrimidine-2,4-dione

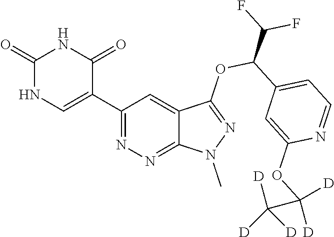 [SMILES: [2H]C([2H])([2H])C([2H])([2H])Oc1cc([C@@H](Oc2nn(C)c3nnc(-c4c[nH]c(=O)[nH]c4=O)cc23)C(F)F)ccn1]

Example 30 was prepared in analogy to Example 10, by replacing 2,2,2-trifluoroethanol with 1,1,1,2,2-pentadeuterio-2-deuteriooxy-ethane in step (b). MS: calc'd 451.2 [(M+H)⁺]; measured 451.4 [(M+H)⁺]. ¹H NMR (400 MHz, DMSO-d₆) δ=11.35 (br s, 2H), 8.72 (s, 1H), 8.38 (s, 1H), 8.19 (d, J=5.3 Hz, 1H), 7.16 (dd, J=5.3, 1.4 Hz, 1H), 6.98 (s, 1H), 6.62 (td, J=54.0, 3.2 Hz, 1H), 6.30-6.18 (m, 1H), 4.02 (s, 3H).

### Example 31

### 5-[3-[(1R)-1-[2-(4,4-difluoro-1-piperidyl)-4-pyridyl]-2,2-difluoro-ethoxy]-1-methyl-pyrazolo[3,4-c]pyridazin-5-yl]-1H-pyrimidine-2,4-dione

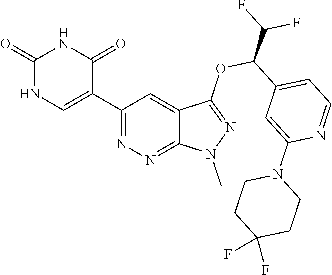 [SMILES: Cn1nc(O[C@H](c2ccnc(N3CCC(F)(F)CC3)c2)C(F)F)c2cc(-c3c[nH]c(=O)[nH]c3=O)nnc21]

Example 31 was prepared in analogy to Example 26, by replacing (2S)-1,1,1-trifluoropropan-2-amine (compound 26.1) with 4,4-difluoropiperidine in step (a). MS: calc'd 521.2 [(M+H)⁺]; measured 521.4 [(M+H)⁺]. ¹H NMR (400 MHz, DMSO-d₆) δ=11.16 (br s, 2H), 8.73 (s, 1H), 8.39 (s, 1H), 8.15 (d, J=5.1 Hz, 1H), 7.16 (s, 1H), 6.86 (d, J=5.1 Hz, 1H), 6.60 (td, J=54.3, 3.0 Hz, 1H), 6.21-6.07 (m, 1H), 4.03 (s, 3H), 3.69 (t, J=5.8 Hz, 4H), 2.06-1.85 (m, 4H).

### Example 32

### 5-[3-[(1R)-1-[2-[(4,4-difluorocyclohexyl)amino]-4-pyridyl]-2,2-difluoro-ethoxy]-1-methyl-pyrazolo[3,4-c]pyridazin-5-yl]-1H-pyrimidine-2,4-dione

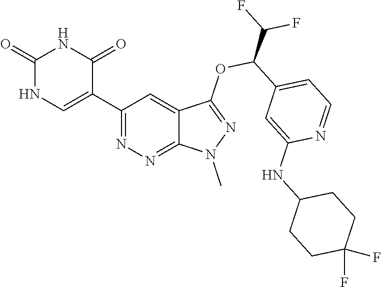 [SMILES: Cn1nc(O[C@H](c2ccnc(NC3CCC(F)(F)CC3)c2)C(F)F)c2cc(-c3c[nH]c(=O)[nH]c3=O)nnc21]

Example 32 was prepared in analogy to Example 26, by replacing (2S)-1,1,1-trifluoropropan-2-amine (compound 26.1) with 4,4-difluorocyclohexanamine in step (a). MS: calc'd 535.2 [(M+H)⁺]; measured 535.3 [(M+H)⁺]. ¹H NMR (400 MHz, DMSO-d₆) δ 11.49 (br s, 2H), 8.71 (s, 1H), 8.40 (s, 1H), 7.99 (d, J=5.3 Hz, 1H), 6.72-6.53 (m, 4H), 6.07 (t, J=11.2 Hz, 1H), 4.03 (s, 3H), 3.89-3.87 (m, 1H), 2.10-1.97 (m, 2H), 1.90 (d, J=13.9 Hz, 4H), 1.48 (d, J=9.6 Hz, 2H).

### Example 33

### 5-[3-[(1R)-1-[2-[(3,3-difluoropyrrolidin-1-yl)methyl]-4-pyridyl]-2,2-difluoro-ethoxy]-1-methyl-pyrazolo[3,4-c]pyridazin-5-yl]-1H-pyrimidine-2,4-dione

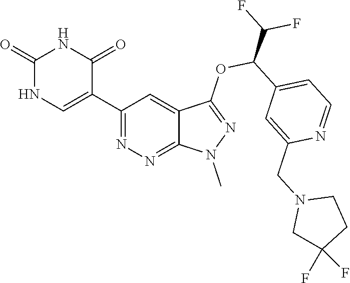 [SMILES: Cn1nc(O[C@H](c2ccnc(CN3CCC(F)(F)C3)c2)C(F)F)c2cc(-c3c[nH]c(=O)[nH]c3=O)nnc21]

The titled compound was synthesized according to the following scheme:

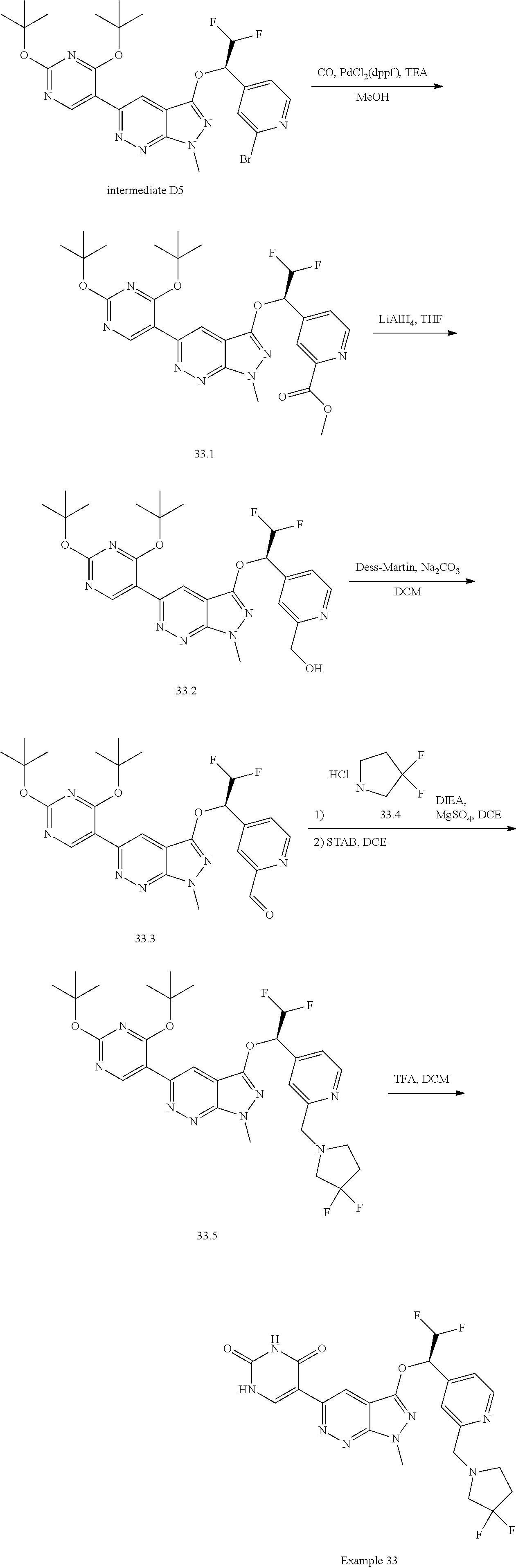

### Step (a): Preparation of methyl 4-[(1R)-1-[5-(2,4-ditert-butoxypyrimidin-5-yl)-1-methyl-pyrazolo[3,4-c]pyridazin-3-yl]oxy-2,2-difluoro-ethyl]pyridine-2-carboxylate (Compound 33.1)

A 30 mL autoclave was charged with 3-[(1R)-1-(2-bromo-4-pyridyl)-2,2-difluoro-ethoxy]-5-(2,4-ditert-butoxypyrimidin-5-yl)-1-methyl-pyrazolo[3,4-c]pyridazine (intermediate D5, 500 mg, 0.84 mmol), Pd(dppf)Cl₂ (61.7 mg, 0.08 mmol), Et₃N (256 mg, 2.53 mmol) and methanol (10 mL) at room temperature. The resultant mixture was stirred at 100° C. for 4 h under carbon monoxide atmosphere (30 atm). After being cooled to room temperature, the reaction mixture was diluted with water (100 mL), and extracted with EA (100 mL) for three times. The combined organic layer was washed with brine, dried over anhydrous Na₂SO₄, filtered and concentrated. The residue was purified by prep-TLC to afford crude compound 33.1 (304 mg). MS: calc'd 572.2 [(M+H)⁺]; measured 572.4 [(M+H)⁺].

### Step (b): Preparation of [4-[(1R)-1-[5-(2,4-ditert-butoxypyrimidin-5-yl)-1-methyl-pyrazolo[3,4-c]pyridazin-3-yl]oxy-2,2-difluoro-ethyl]-2-pyridyl]methanol (compound 33.2)

To a solution of methyl 4-[(1R)-1-[5-(2,4-ditert-butoxypyrimidin-5-yl)-1-methyl-pyrazolo[3,4-c]pyridazin-3-yl]oxy-2,2-difluoro-ethyl]pyridine-2-carboxylate (compound 33.1, 200 mg, 0.35 mmol) in THF (10.0 mL) was added dropwise lithium aluminum hydride (2 M in THF, 0.35 mL, 0.7 mmol) at −78° C. After being stirred at −78° C. for 2 h, the reaction was quenched with water (20 mL) at −78° C., warmed to room temperature, and extracted with EA (50 mL) for three times. The combined organic layer was dried over anhydrous Na₂SO₄, filtered and concentrated. The residue was purified by Prep-TLC to afford compound 33.2 (93.0 mg). MS: calc'd 544.2 [(M+H)⁺]; measured 544.2 [(M+H)⁺].

### Step (c): Preparation of 4-[(1R)-1-[5-(2,4-ditert-butoxypyrimidin-5-yl)-1-methyl-pyrazolo[3,4-c]pyridazin-3-yl]oxy-2,2-difluoro-ethyl]pyridine-2-carbaldehyde (compound 33.3)

To a solution of [4-[(1R)-1-[5-(2,4-ditert-butoxypyrimidin-5-yl)-1-methyl-pyrazolo[3,4-c]pyridazin-3-yl]oxy-2,2-difluoro-ethyl]-2-pyridyl]methanol (compound 33.2, 93 mg, 0.17 mmol) in DCM (5 mL) was added Na₂CO₃ (53.4 mg, 0.51 mmol) and DMP (109 mg, 0.26 mmol). The resultant mixture was stirred at room temperature for 2 h. The reaction was quenched with water (30 mL), and extracted with EA (50 mL) for three times. The combined organic layer was dried over anhydrous Na₂SO₄, filtered and concentrated. The residue was purified by Prep-TLC to afford compound 33.3 (30.0 mg). MS: calc'd 542.2 [(M+H)⁺]; measured 542.2 [(M+H)⁺].

### Step (d): Preparation of 5-(2,4-ditert-butoxypyrimidin-5-yl)-3-[(1R)-1-[2-[(3,3-difluoroazetidin-1-yl)methyl]-4-pyridyl]-2,2-difluoro-ethoxy]-1-methyl-pyrazolo[3,4-c]pyridazine (compound 33.5)

To a solution of 4-[(1R)-1-[5-(2,4-ditert-butoxypyrimidin-5-yl)-1-methyl-pyrazolo[3,4-c]pyridazin-3-yl]oxy-2,2-difluoro-ethyl]pyridine-2-carbaldehyde (compound 33.3, 40.0 mg, 0.07 mmol) in DCE (5 mL) was added 3,3-difluoropyrrolidine hydrochloride (compound 33.4, 12.7 mg, 0.09 mmol), MgSO₄ (17.8 mg, 0.15 mmol) and DIEA (28.6 mg, 0.22 mmol). The resultant mixture was stirred at room temperature for 3 h. Then STAB (62.6 mg, 0.3 mmol) was added, and the reaction mixture was stirred at room temperature for another 1 h. The reaction was quenched with water (30 mL), and extracted with EA (50 mL) for three times. The combined organic layer was dried over anhydrous Na₂SO₄, filtered and concentrated. The residue was purified by Prep-TLC to afford compound 33.5 (31.0 mg). MS: calc'd 633.3 [(M+H)⁺]; measured 655.2 [(M+Na)⁺].

### Step (e): Preparation of 5-[3-[(1R)-1-[2-[(3,3-difluoropyrrolidin-1-yl)methyl]-4-pyridyl]-2,2-difluoro-ethoxy]-1-methyl-pyrazolo[3,4-c]pyridazin-5-yl]-1H-pyrimidine-2,4-dione (Example 33)

To a solution of 5-(2,4-ditert-butoxypyrimidin-5-yl)-3-[(1R)-1-[2-[(3,3-difluoropyrrolidin-1-yl)methyl]-4-pyridyl]-2,2-difluoro-ethoxy]-1-methyl-pyrazolo[3,4-c]pyridazine (compound 33.5, 30.0 mg, 0.05 mmol) in DCM (5 mL) was added TFA (0.5 mL). The resultant mixture was stirred at room temperature for 1 h, then concentrated to afford a crude product, which was purified by pre-HPLC to afford example 33 (12.1 mg). MS: calc'd 521.2 [(M+H)⁺]; measured 521.2 [(M+H)⁺]. ¹H NMR (400 MHz, DMSO-d₆) δ=11.22 (br s, 2H), 8.73 (s, 1H), 8.56 (d, J=5.1 Hz, 1H), 8.39 (s, 1H), 7.62 (d, J=1.6 Hz, 1H), 7.53-7.48 (m, 1H), 6.66 (td, J=53.9, 2.9 Hz, 1H), 6.30 (td, J=11.9, 2.9 Hz, 1H), 4.00 (s, 3H), 3.78 (d, J=1.5 Hz, 2H), 3.00-2.78 (m, 2H), 2.79-2.63 (m, 2H), 2.08-2.29 (m, 2H).

### Example 34

### 5-[3-[(1R)-2,2-difluoro-1-[2-(2,2,2-trifluoroethoxymethyl)-4-pyridyl]ethoxy]-1-methyl-pyrazolo[3,4-c]pyridazin-5-yl]-1H-pyrimidine-2,4-dione

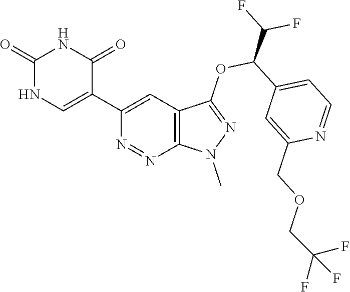 [SMILES: Cn1nc(O[C@H](c2ccnc(COCC(F)(F)F)c2)C(F)F)c2cc(-c3c[nH]c(=O)[nH]c3=O)nnc21]

The titled compound was synthesized according to the following scheme:

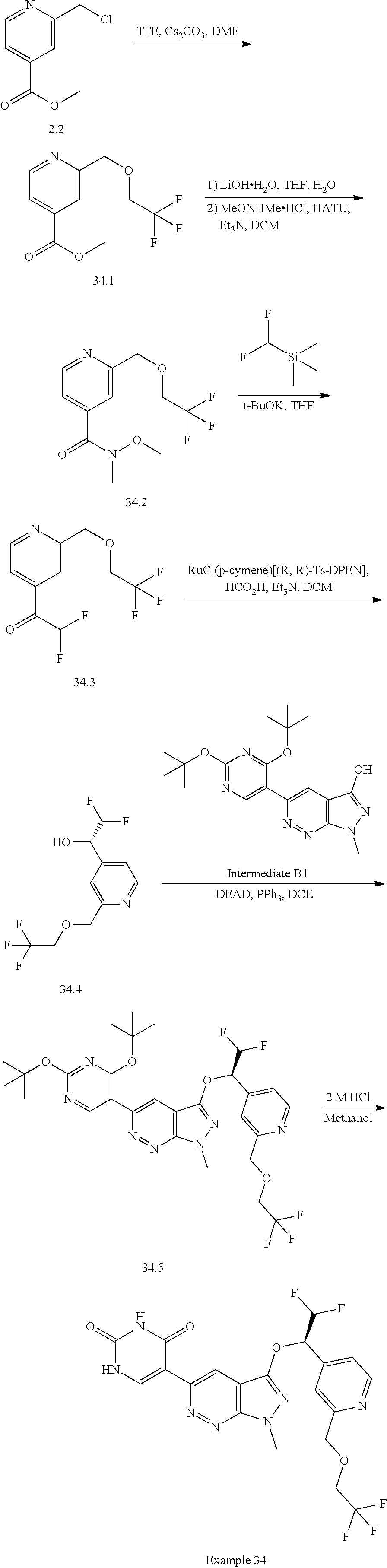

### Step (a): Preparation of methyl 2-(2,2,2-trifluoroethoxymethyl)pyridine-4-carboxylate (Compound 34.1)

To a solution of methyl 2-(chloromethyl)pyridine-4-carboxylate (compound 2.1, 5.55 g, 29.9 mmol) in DMF (50 mL) was added 2,2,2-trifluoroethanol (6.0 mL, 83.4 mmol) and Cs₂CO₃ (24.4 g, 74.8 mmol) at 22° C. The resultant mixture was stirred at 60° C. for 16 h. The reaction mixture was diluted with saturated aqueous NaCl (200 mL), and extracted with EA (200 mL) for three times. The combined organic layer was washed with brine, dried over Na₂SO₄, filtered and concentrated in vacuo. The residue was purified by flash chromatography (silica gel, 120 g, 10% to 30% EA in PE) to afford compound 34.1 (3.87 g). MS: calc'd 250.1 [(M+H)⁺]; measured 249.9 [(M+H)⁺].

### Step (b): Preparation of N-methoxy-N-methyl-2-(2,2,2 trifluoroethoxymethyl)pyridine-4-carboxamide (Compound 34.2)

To a solution of methyl 2-(2,2,2-trifluoroethoxymethyl)pyridine-4-carboxylate (compound 34.1, 3.87 g, 15.5 mmol) in THF (50 mL) was added H₂O (50 mL) and LiOH·H₂O (716.8 mg, 17.1 mmol) at 22° C. The resultant mixture was stirred at 22° C. for 2 h. HCl aqueous solution (2.0 M) was added to adjust the pH value to 4. The reaction mixture was extracted with EA (100 mL) for three times. The combined organic layer was washed with brine, dried over Na₂SO₄, filtered and concentrated in vacuo. The resultant residue was suspended in DCM, and N,O-dimethylhydroxylamine hydrochloride (1.82 g, 18.6 mmol), Et₃N (4.0 mL, 28.7 mmol) and HATU (6.47 g, 17.1 mmol) were added at 22° C. The resultant mixture was stirred at 22° C. for 2 hr. The reaction mixture was diluted with saturated aqueous NaHCO₃ (200 mL), and extracted with DCM (200 mL) twice. The combined organic layer was washed with brine, dried over Na₂SO₄, filtered and concentrated in vacuo. The residue was purified by flash chromatography (silica gel, 80 g, 30% to 70% EA in PE) to afford compound 34.2 (3.55 g). MS: calc'd 279.1 [(M+H)⁺]; measured 279.0 [(M+H)⁺].

### Step (c): Preparation of 2,2-difluoro-1-[2-(2,2,2-trifluoroethoxymethyl)-4-pyridyl]ethanone (compound 34.3)

To a solution of N-methoxy-N-methyl-2-(2,2,2-trifluoroethoxymethyl)pyridine-4-carboxamide (compound 34.2, 3.55 g, 12.8 mmol) in THF (30 mL) was added difluoromethyltrimethylsilane (2.5 mL, 27.4 mmol) and potassium tert-butoxide (2.15 g, 19.1 mmol) at 0° C. The resultant mixture was stirred at 0° C. for 2 h. The reaction mixture was diluted with saturated aqueous NaCl (100 mL), and extracted with EA (100 mL) for three times. The combined organic layer was washed with brine, dried over Na₂SO₄, filtered and concentrated in vacuo. The residue was purified by flash chromatography (silica gel, 80 g, 0% to 8% MeOH in DCM) to afford compound 34.3 (2.67 g). MS: calc'd 288.1 [(M+H₂O+H)⁺]; measured 287.9 [(M+H₂O+H)⁺].

### Step (d): Preparation of (1S)-2,2-difluoro-1-[2-(2,2,2-trifluoroethoxymethyl)-4-pyridyl]ethanol (compound 34.4)

To a solution of 2,2-difluoro-1-[2-(2,2,2-trifluoroethoxymethyl)-4-pyridyl]ethanone (compound 34.3, 2.67 g, 9.92 mmol) in DCM (10 mL) was added formic acid (2.0 mL, 52.1 mmol), triethylamine (3.0 mL, 21.5 mmol), and RuCl(p-cymene)[(R,R)-Ts-DPEN](315.5 mg, 496.0 μmol) at 0° C. The resultant mixture was stirred at 22° C. for 2 h. The reaction mixture was diluted with saturated aqueous NaHCO₃ (100 mL), and extracted with DCM (100 mL) for three times. The combined organic layer was washed with brine, dried over Na₂SO₄, filtered and concentrated in vacuo. The residue was purified by flash chromatography (silica gel, 80 g, 25% to 50% EA in PE) to afford compound 34.4 (1.46 g). MS: calc'd 272.1 [(M+H)⁺]; measured 271.9 [(M+H)⁺].

### Step (e): Preparation of 5-(2,4-ditert-butoxypyrimidin-5-yl)-3-[(1R)-2,2-difluoro-1-[2-(2,2,2-trifluoroethoxymethyl)-4-pyridyl]ethoxy]-1-methyl-pyrazolo[3,4-c]pyridazine (Compound 34.5)

To a suspension 5-(2,4-ditert-butoxypyrimidin-5-yl)-1-methyl-pyrazolo[3,4-c]pyridazin-3-ol (intermediate B1, 1.20 g, 3.22 mmol) in DCE (26 mL) was added (1S)-2,2-difluoro-1-[2-(2,2,2-trifluoroethoxymethyl)-4-pyridyl]ethanol (compound 34.4, 1.22 g, 4.51 mmol), triphenylphosphine (1.23 g, 4.67 mmol) and DEAD (740 μL, 4.67 mmol) at 60° C. The resultant mixture was stirred at 60° C. for 1 h. After being cooled to room temperature, the reaction mixture was diluted with saturated aqueous NaHCO₃ (100 mL), and extracted with DCM (100 mL) for three times. The combined organic layer was washed with brine, dried over Na₂SO₄, filtered and concentrated in vacuo. The residue was purified by flash chromatography (silica gel, 80 g, 10% to 40% EA in PE) to afford compound 34.5 (1.76 g). MS: calc'd 514.1 [(M−112+H)⁺]; measured 514.1 [(M−112+H)⁺].

### Step (f): Preparation of 5-[3-[(1R)-2,2-difluoro-1-[2-(2,2,2-trifluoroethoxymethyl)-4-pyridyl]ethoxy]-1-methyl-pyrazolo[3,4-c]pyridazin-5-yl]-1H-pyrimidine-2,4-dione (Example 34)

To the mixture of 5-(2,4-ditert-butoxypyrimidin-5-yl)-3-[(1R)-2,2-difluoro-1-[2-(2,2,2-trifluoroethoxymethyl)-4-pyridyl]ethoxy]-1-methyl-pyrazolo[3,4-c]pyridazine (compound 34.5, 660.0 mg, 1.06 mmol) in methanol (6 mL) was added HCl (2.0 M in MeOH, 2.1 mL, 4.2 mmol). The reaction mixture was stirred at room temperature for 30 min, then concentrated to afford a crude product, which was purified by pre-HPLC to afford Example 34 (353.1 mg). MS: calc'd 514.1 [(M+H)⁺]; measured 514.1 [(M+H)⁺]. ¹H NMR (400 MHz, DMSO-d₆) δ=11.55 (d, J=1.6 Hz, 1H), 11.45 (br d, J=5.8 Hz, 1H), 8.72 (s, 1H), 8.60 (d, J=5.1 Hz, 1H), 8.38 (d, J=6.2 Hz, 1H), 7.65 (s, 1H), 7.56 (d, J=5.1 Hz, 1H), 6.65 (td, J=53.7, 2.8 Hz, 1H), 6.33 (td, J=11.8, 2.3 Hz, 1H), 4.77 (s, 2H), 4.28-4.11 (m, 2H), 4.01 (s, 3H).

### Example 35A and Example 35B

### 5-[3-[(1R)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione and 5-[3-[(1S)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione

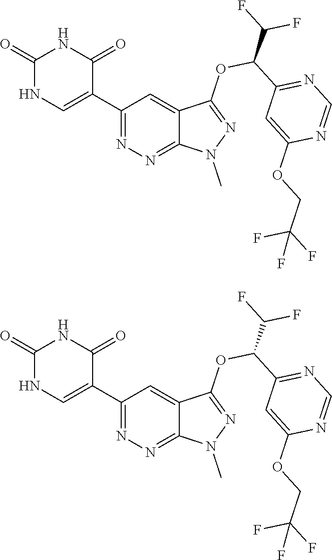 [SMILES: Cn1nc(O[C@H](c2cc(OCC(F)(F)F)ncn2)C(F)F)c2cc(-c3c[nH]c(=O)[nH]c3=O)nnc21 ; Cn1nc(O[C@@H](c2cc(OCC(F)(F)F)ncn2)C(F)F)c2cc(-c3c[nH]c(=O)[nH]c3=O)nnc21]

The titled compound was synthesized according to the following scheme:

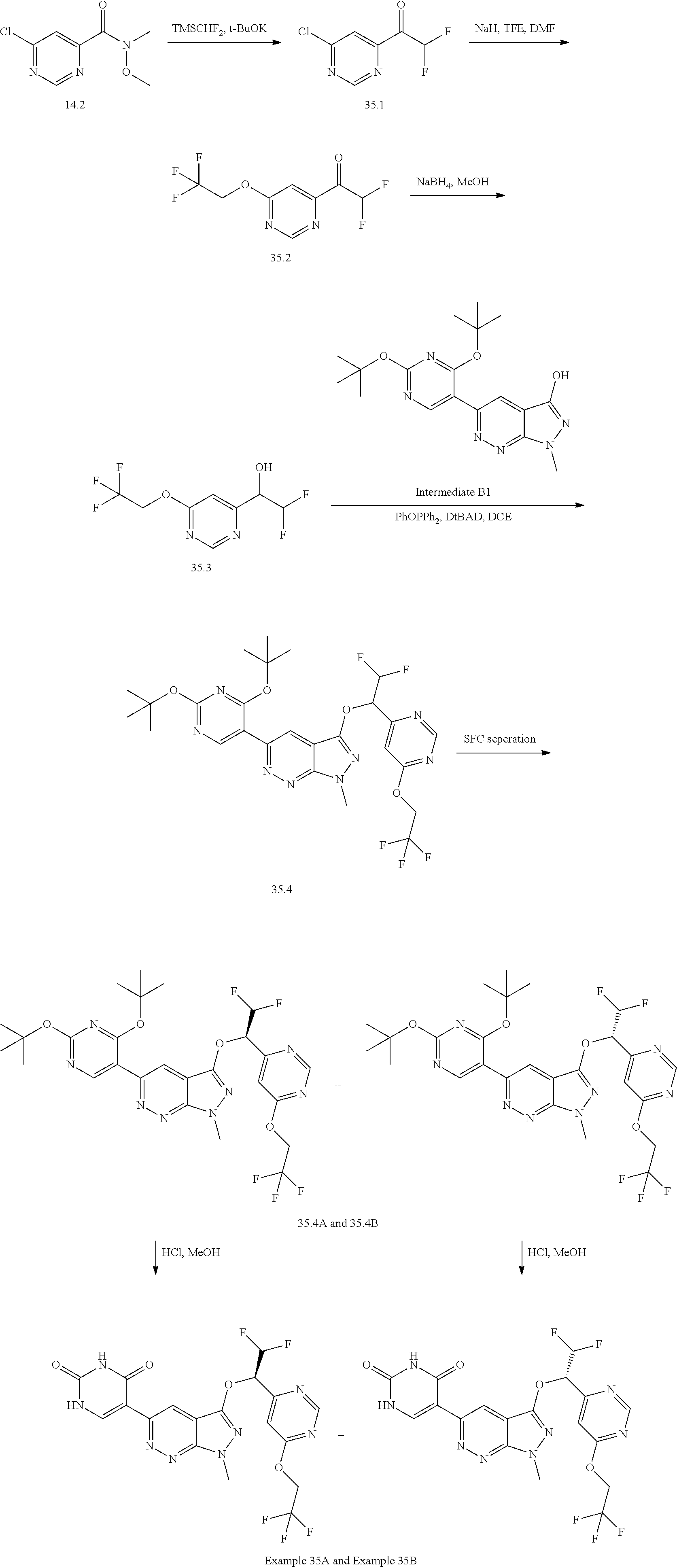

### Step (a): Preparation of 1-(6-chloropyrimidin-4-yl)-2,2-difluoro-ethanone (Compound 35.1)

To a solution of methyl 6-chloro-N-methoxy-N-methyl-pyrimidine-4-carboxamide (compound 14.2, 6.0 g, 29.8 mmol) in THF (100 mL) was added TMSCHF₂ (8.0 mL, 56.5 mmol) and t-BuOK (4.01 g, 35.7 mmol) at 0° C. The resultant mixture was stirred at 0° C. for 30 min. The reaction mixture was diluted with saturated aqueous NaHCO₃ (200 mL), and extracted with EA (200 mL) for three times. The combined organic layer was washed with brine, dried over Na₂SO₄, filtered and concentrated in vacuo. The residue was purified by flash chromatography (silica gel, 120 g, 0% to 30% EA in PE) to afford compound 35.1 (3.13 g). MS: calc'd 193.0 [(M+H)⁺]; measured 210.9 [(M+H₂O+H)⁺].

### Step (b): Preparation of 2,2-difluoro-1-[6-(2,2,2-trifluoroethoxy)pyrimidin-4-yl]ethanone (compound 35.2)

To a suspension of NaH (60% dispersion in mineral oil, 4.15 g, 103.9 mmol) in DMF (100 mL) was added 2,2,2-trifluoroethanol (9.0 mL, 125.1 mmol) at 0° C. The resultant mixture was stirred at 0° C. for 10 min, and then 1-(6-chloropyrimidin-4-yl)-2,2-difluoro-ethanone (compound 35.1, 10.0 g, 51.9 mmol) was added. The resultant mixture was stirred at 22° C. for 2 h. The reaction mixture was diluted with saturated aqueous NaCl (400 mL), and extracted with EA (200 mL) for three times. The combined organic layer was washed with brine, dried over Na₂SO₄, filtered and concentrated in vacuo. The residue was purified by flash chromatography (silica gel, 120 g, 0% to 35% EA in PE) to afford compound 35.2 (7.16 g). MS: calc'd 257.0 [(M+H)⁺]; measured 274.9 [(M+H₂O+H)⁺].

### Step (c): Preparation of 2,2-difluoro-1-[6-(2,2,2-trifluoroethoxy)pyrimidin-4-yl]ethanol (compound 35.3)

To a solution of 2,2-difluoro-1-[6-(2,2,2-trifluoroethoxy)pyrimidin-4-yl]ethanone (compound 35.2, 3.62 g, 14.1 mmol) in MeOH (30 mL) was added NaBH₄ (641.6 mg, 17.0 mmol) at 0° C. The resultant mixture was stirred at 0° C. for 30 min. The reaction mixture was diluted with saturated aqueous NaCl (200 mL), and extracted with EA (100 mL) for three times. The combined organic layer was washed with brine, dried over Na₂SO₄, filtered and concentrated in vacuo. The residue was purified by flash chromatography (silica gel, 80 g, 0% to 35% EA in PE) to afford compound 35.3 (3.27 g). MS: calc'd 259.1 [(M+H)⁺]; measured 258.9 [(M+H)⁺].

### Step (d): Preparation of 5-(2,4-ditert-butoxypyrimidin-5-yl)-3-[2,2-difluoro-1-[6-(2,2,2-trifluoroethoxy)pyrimidin-4-yl]ethoxy]-1-methyl-pyrazolo[3,4-c]pyridazine (Compound 35.4)

To a suspension of 5-(2,4-ditert-butoxypyrimidin-5-yl)-1-methyl-pyrazolo[3,4-c]pyridazin-3-ol (intermediate B1, 900 mg, 2.42 mmol) in DCE (24 mL) was added 2,2-difluoro-1-[6-(2,2,2-trifluoroethoxy)pyrimidin-4-yl]ethanol (compound 35.3, 1.87 g, 7.25 mmol), phenoxy(diphenyl)phosphane (2.69 g, 9.67 mmol) and DtBAD (2.23 g, 9.67 mmol) at 60° C. The resultant mixture was stirred at 60° C. for 1 h. After being cooled to room temperature, the reaction mixture was diluted with saturated aqueous NaCl (100 mL), and extracted with DCM (100 mL) for three times. The combined organic layer was washed with brine, dried over Na₂SO₄, filtered and concentrated in vacuo. The residue was purified by flash chromatography (silica gel, 80 g, 10% to 30% EA in PE) to afford compound 35.4 (800 mg). MS: calc'd 513.2 [(M+H)⁺]; measured 501.1 [(M−112+H)⁺].

### Step (e): Preparation of 5-(2,4-ditert-butoxypyrimidin-5-yl)-3-[(1R)-2,2-difluoro-1-[6-(2,2,2-trifluoroethoxy)pyrimidin-4-yl]ethoxy]-1-methyl-pyrazolo[3,4-c]pyridazine and 5-(2,4-ditert-butoxypyrimidin-5-yl)-3-[(1S)-2,2-difluoro-1-[6-(2,2,2-trifluoroethoxy)pyrimidin-4-yl]ethoxy]-1-methyl-pyrazolo[3,4-c]pyridazine (compound 35.4A and 35.4B)

Compound 35.4 (800 mg) was resolved by SFC to give two single isomers: compound 35.4A (faster eluting, 190 mg) MS: calc'd 501.1 [(M−112+H)⁺]; measured 501.1 [(M−112+H)⁺]; and compound 35.4B (slower eluting, 142 mg) MS: calc'd 501.1 [(M−112+H)⁺]; measured 501.1 [(M−112+H)⁺]. SFC conditions: Column: ID 250×20 mm I.D. 5 μm, mobile phase: A for CO₂ and B for EtOH (0.1% NH₃·H₂O), gradient: B 10%, flow rate: 60 mL/min, back pressure: 100 bar, Column temperature: 35° C.

### Step (f): Preparation of 5-[3-[(1R)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione and 5-[3-[(1S)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione (Example 35A and Example 35B)

To a solution of compound 35.4A (263.7 mg, 430.5 μmol) in methanol (5 mL) was added HCl (2.0 M in MeOH, 600 μL, 1.2 mmol). The resultant mixture was stirred at room temperature for 1 h, then concentrated to afford a crude product, which was purified by pre-HPLC to afford Example 35A (84.9 mg). MS: calc'd 501.1 [(M+H)⁺]; measured 501.0 [(M+H)⁺]. ¹H NMR (400 MHz, DMSO-d₆) δ=11.55 (s, 1H), 11.46 (br d, J=5.9 Hz, 1H), 8.94 (s, 1H), 8.73 (s, 1H), 8.38 (d, J=6.3 Hz, 1H), 7.42 (s, 1H), 6.74 (td, J=53.3, 2.6 Hz, 1H), 6.34-6.14 (m, 1H), 5.17-5.05 (m, 2H), 4.02 (s, 3H).

To a solution of compound 35.4B (182.5 mg, 297.9 μmol) in methanol (4 mL) was added HCl (2.0 M in MeOH, 400 μL, 800 μmol). The resultant mixture was stirred at room temperature for 1 h, then concentrated to afford a crude product, which was purified by pre-HPLC to afford Example 35B (89.4 mg). MS: calc'd 501.1 [(M+H)⁺]; measured 501.0 [(M+H)⁺]. ¹H NMR (400 MHz, DMSO-d₆) δ=11.55 (br s, 1H), 11.46 (br d, J=4.8 Hz, 1H), 8.94 (s, 1H), 8.73 (s, 1H), 8.38 (d, J=6.0 Hz, 1H), 7.42 (s, 1H), 6.74 (t, J=52.8 Hz, 1H), 6.31-6.16 (m, 1H), 5.11 (q, J=8.8 Hz, 2H), 4.02 (s, 3H).

### Example 36A and Example 36B

### 5-[3-[(1R)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione and 5-[3-[(1S)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione

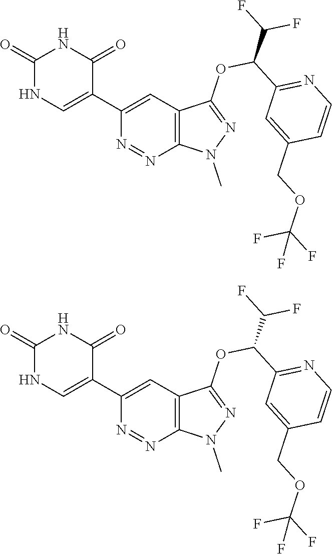 [SMILES: Cn1nc(O[C@H](c2cc(COC(F)(F)F)ccn2)C(F)F)c2cc(-c3c[nH]c(=O)[nH]c3=O)nnc21 ; Cn1nc(O[C@@H](c2cc(COC(F)(F)F)ccn2)C(F)F)c2cc(-c3c[nH]c(=O)[nH]c3=O)nnc21]

The titled compound was synthesized according to the following scheme:

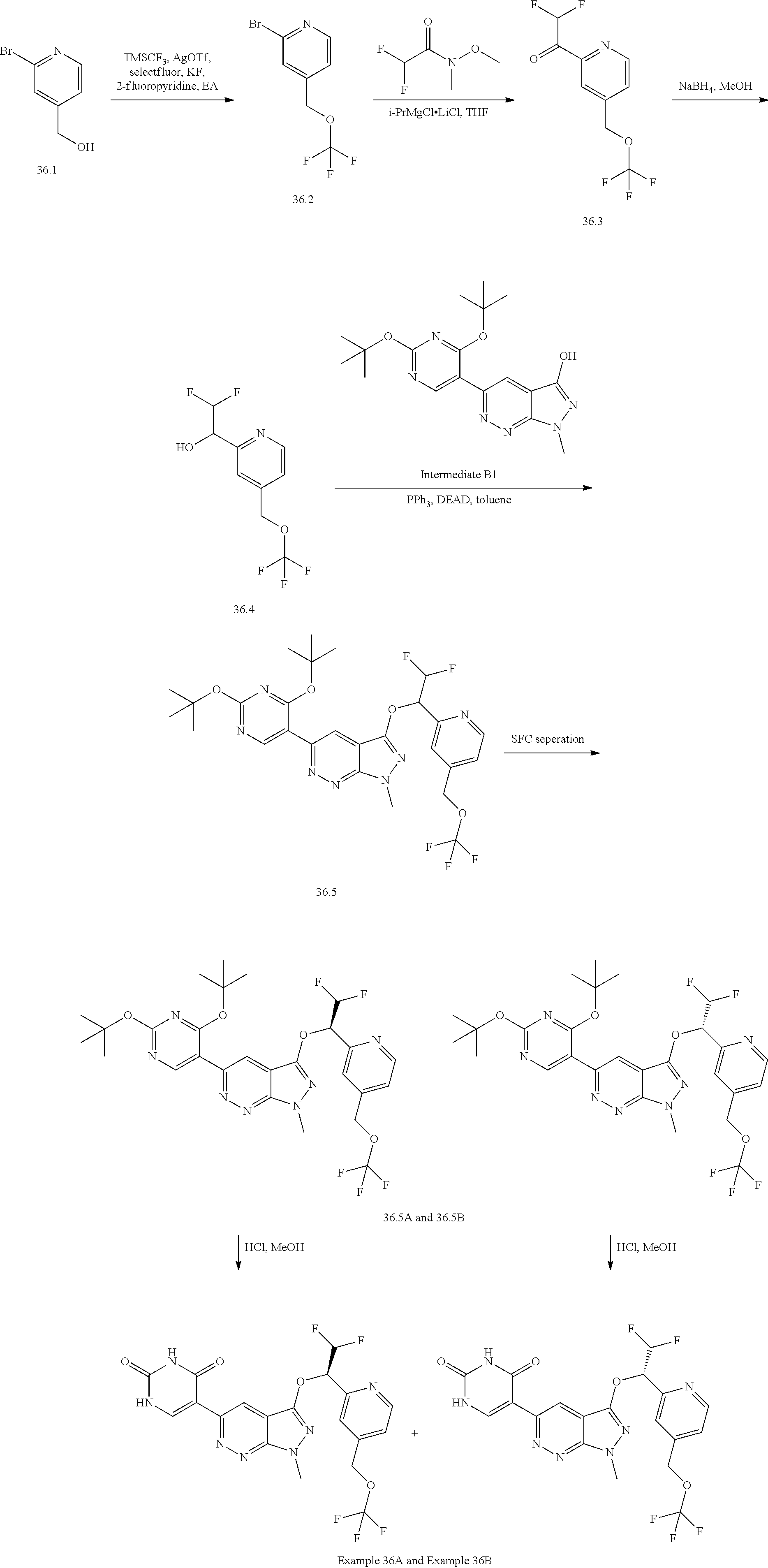

### Step (a): Preparation of 1-(6-chloropyrimidin-4-yl)-2,2-difluoro-ethanone (Compound 36.2)

To a solution of (2-bromo-4-pyridyl)methanol (compound 36.1, 6.0 g, 31.9 mmol) in LA (60 mL) was added selectfluor (17.0 g, 47.9 mmol), KF (7.42 g, 127.6 mmol), AgOTf (24.6 g, 95.7 mmol), 2-fluoropyridine (9.0 mL, 104.8 mmol) and TMSCF₃ (15.0 mL, 93.9 mmol) at 22° C. The resultant mixture was stirred at 22° C. for 24 h. The reaction mixture was diluted with saturated aqueous NaHCO₃ (200 mL), and extracted with LA (200 mL) for three times. The combined organic layer was washed with brine, dried over Na₂SO₄, filtered and concentrated in vacuo. The residue was purified by flash chromatography (silica gel, 120 g. 0% to 12% LA in PE) to afford compound 36.2 (957.0 mg). MS: calc'd 256.0, 258.0 [(M+H)⁺]; measured 255.8, 257.8 [(M+H)⁺].

### Step (b): Preparation of 2,2-difluoro-1-[4-(trifluoromethoxymethyl)-2-pyridyl]ethanone (compound 36.3)

To a solution of 1-(6-chloropyrimidin-4-yl)-2,2-difluoro-ethanone (compound 36.2, 697.3 mg, 2.72 mmol) in THF (10 mL) was added i-PrMgCl·LiCl (1.3 M in THF, 10 mL, 13.0 mmol) at 0° C. The resultant mixture was stirred at 22° C. for 30 min, and then 2,2-difluoro-N-methoxy-N-methylacetamide (1.5 mL, 12.9 mmol) was added. The reaction mixture was stirred at 22° C. for 1 h. The reaction mixture was diluted with saturated aqueous NH₄Cl (50 mL), and extracted with EA (50 mL) for three times. The combined organic layer was washed with brine, dried over Na₂SO₄, filtered and concentrated in vacuo. The residue was purified by flash chromatography (silica gel, 24 g, 0% to 25% EA in PE) to afford compound 36.3 (372.0 g). MS: calc'd 258.0 [(M+H)⁺]; measured 273.9 [(M+H₂O+H)⁺].

### Step (c): Preparation of 2,2-difluoro-1-[4-(trifluoromethoxymethyl)-2-pyridyl]ethanol (Compound 36.4)

To a solution of 2,2-difluoro-1-[4-(trifluoromethoxymethyl)-2-pyridyl]ethanone (compound 36.3, 372.0 mg, 1.46 mmol) in MeOH (6 mL) was added NaBH₄ (165.5 mg, 4.37 mmol) at 0° C. The resultant mixture was stirred at 0° C. for 30 min. The reaction mixture was diluted with saturated aqueous NaCl (50 mL), and extracted with EA (50 mL) for three times. The combined organic layer was washed with brine, dried over Na₂SO₄, filtered and concentrated in vacuo. The residue was purified by flash chromatography (silica gel, 24 g, 0% to 30% EA in PE) to afford compound 36.4 (233.0 mg). MS: calc'd 258.1 [(M+H)⁺]; measured 257.9 [(M+H)⁺].

### Step (d): Preparation of 5-(2,4-ditert-butoxypyrimidin-5-yl)-3-[2,2-difluoro-1-[4-(trifluoromethoxymethyl)-2-pyridyl]ethoxy]-1-methyl-pyrazolo[3,4-c]pyridazine (Compound 36.5)

To a suspension of 5-(2,4-ditert-butoxypyrimidin-5-yl)-1-methyl-pyrazolo[3,4-c]pyridazin-3-ol (intermediate B1, 500.0 mg, 1.34 mmol) in toluene (12 mL) was added 2,2-difluoro-1-[4-(trifluoromethoxymethyl)-2-pyridyl]ethanol (compound 36.4, 517.9 mg, 2.01 mmol), triphenylphosphine (528.2 mg, 2.01 mmol) and DEAD (340 μL, 2.15 mmol) at 60° C. The resultant mixture was stirred at 60° C. for 1 h. After being cooled to room temperature, the reaction mixture was diluted with saturated aqueous NaCl (50 mL), and extracted with EA (50 mL) for three times. The combined organic layer was washed with brine, dried over Na₂SO₄, filtered and concentrated in vacuo. The residue was purified by flash chromatography (silica gel, 80 g, 10% to 40% EA in PE) to afford compound 36.5 (700 mg). MS: calc'd 612.1 [(M+H)⁺]; measured 500.1 [(M−112+H)⁺].

### Step (e): Preparation of 5-(2,4-ditert-butoxypyrimidin-5-yl)-3-[(1R)-2,2-difluoro-1-[4-(trifluoromethoxymethyl)-2-pyridyl]ethoxy]-1-methyl-pyrazolo[3,4-c]pyridazine and 5-(2,4-ditert-butoxypyrimidin-5-yl)-3-[(1S)-2,2-difluoro-1-[4-(trifluoromethoxymethyl)-2-pyridyl]ethoxy]-1-methyl-pyrazolo[3,4-c]pyridazine (compound 36.5A and 36.5B)

Compound 36.5 (700 mg) was resolved by SFC to give two single isomers: compound 36.5A (faster eluting, 318 mg) MS: calc'd 500.1 [(M−112+H)⁺]; measured 500.1 [(M−112+H)⁺]; and compound 36.5B (slower eluting, 303 mg) MS: calc'd 500.1 [(M−112+H)⁺]; measured 500.1 [(M−112+H)⁺]. SFC conditions: Column: OX 250×30 mm I.D. 5 μm, mobile phase: A for CO₂ and B for i-PrOH (0.1% NH₃·H₂O), gradient: B 50%, flow rate: 60 mL/min, back pressure: 100 bar, Column temperature: 35° C.

### Step (f): Preparation of 5-[3-[(1R)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione and 5-[3-[(1S)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione (Example 36A and Example 36B)

To a solution of compound 36.5A (318.0 mg, 520.0 μmol) in methanol (5 mL) was added HCl (2.0 M in MeOH, 700 μL, 1.4 mmol). The resultant mixture was stirred at room temperature for 1 hr, then concentrated to afford a crude product, which was purified by pre-HPLC to afford Example 36A (210.4 mg). MS: calc'd 500.1 [(M+H)⁺]; measured 500.0 [(M+H)⁺]. ¹H NMR (400 MHz, DMSO-d₆) δ=11.54 (s, 1H), 11.45 (br d, J=5.5 Hz, 1H), 8.70 (s, 1H), 8.69 (d, J=4.9 Hz, 1H), 8.38 (d, J=6.3 Hz, 1H), 7.70 (s, 1H), 7.46 (d, J=5.4 Hz, 1H), 6.72 (td, J=53.4, 3.6 Hz, 1H), 6.31-6.20 (m, 1H), 5.29 (s, 2H), 4.01 (s, 3H).

To a solution of compound 36.5B (303.0 mg, 495.4 μmol) in methanol (5 mL) was added HCl (2.0 M in MeOH, 700 μL, 1.4 mmol). The resultant mixture was stirred at room temperature for 1 hr, then concentrated to afford a crude product, which was purified by pre-HPLC to afford Example 36B (213.3 mg). MS: calc'd 500.1 [(M+H)⁺]; measured 500.0 [(M+H)⁺]. ¹H NMR (500 MHz, DMSO-d₆) δ=11.56 (d, J=1.7 Hz, 1H), 11.46 (dd, J=6.0, 1.4 Hz, 1H), 8.71 (s, 1H), 8.69 (d, J=5.0 Hz, 1H), 8.38 (d, J=6.1 Hz, 1H), 7.70 (s, 1H), 7.53-7.37 (m, 1H), 6.73 (td, J=53.8, 3.1 Hz, 1H), 6.26 (ddd, J=14.0, 8.9, 3.2 Hz, 1H), 5.29 (s, 2H), 4.01 (s, 3H).

### Example 37A and 37B

### 5-[3-[(1R)-1-[2-[(1S)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione and 5-[3-[(1R)-1-[2-[(1R)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione

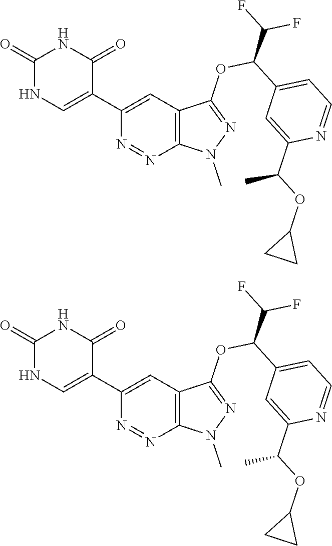

The titled compounds were synthesized according to the following scheme:

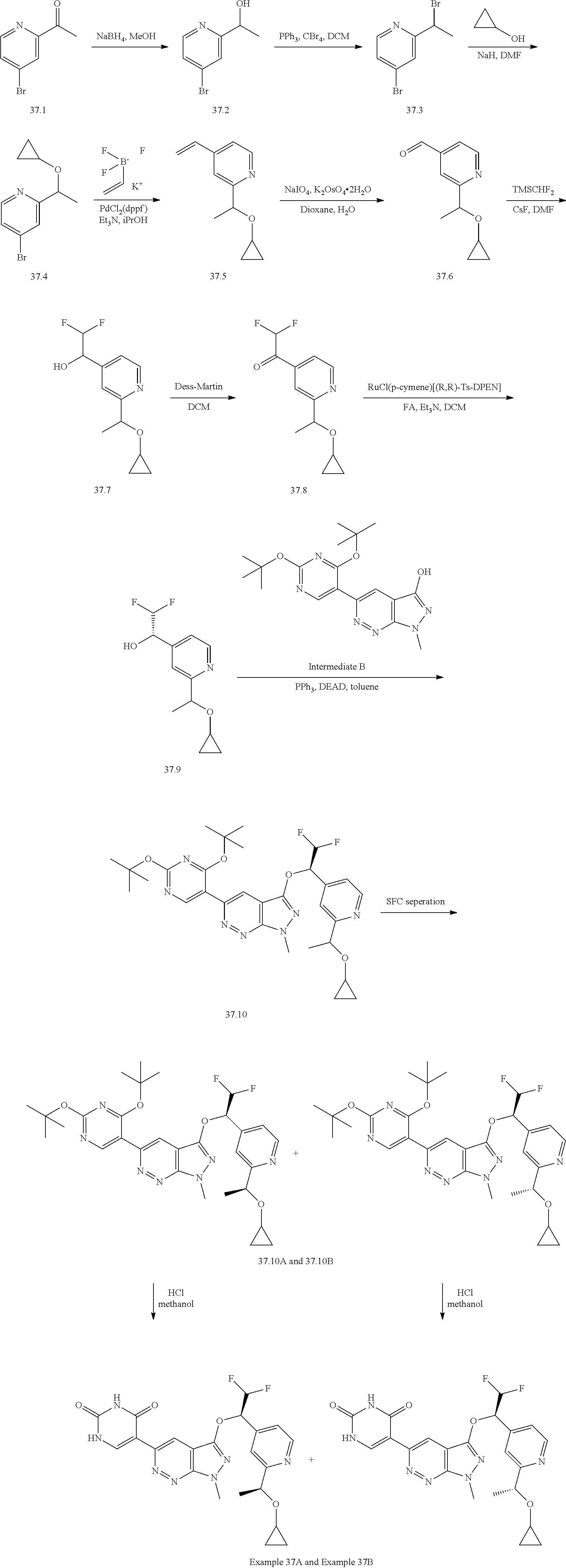

### Step (a): Preparation of 1-(4-bromo-2-pyridyl)ethanol (compound 37.2)

To a solution of 1-(4-bromo-2-pyridyl)ethanol (compound 37.1, 5 g, 25 mmol) in methanol (50 mL) was added sodium borohydride (1.1 g, 30 mmol) at 0° C. The resultant mixture was stirred at 0° C. for 0.5 hr. The reaction was quenched with 1 N HCl. Methanol was removed under vacuum, and the resultant mixture was extracted with DCM (20 mL) for three times. The combined organic layer was washed with brine, dried over Na₂SO₄, filtered and concentrated in vacuo to afford compound 37.2 (4.8 g). calc'd 201.9 [(M+H)⁺], measured 201.9 [(M+H)⁺].

### Step (b): Preparation of 4-bromo-2-(1-bromoethyl)pyridine (Compound 37.3)

To the suspension of 1-(4-bromo-2-pyridyl)ethanol (compound 37.2, 4.8 g, 23.8 mmol) in DCM (60 mL) was added carbon tetrabromide (10.2 g, 30.9 mmol) and triphenylphosphine (8.1 g, 30.9 mmol) at room temperature. The resultant mixture was stirred at room temperature for 16 h. The reaction mixture was concentrated in vacuo, and the residue was purified by silica gel chromatography (PE/EA from 100/1 to 10/1) to afford compound 37.3 (6 g). calc'd 263.9 [(M+H)⁺], measured 263.9 [(M+H)⁺].

### Step (c): Preparation of 4-bromo-2-[1-(cyclopropoxy)ethyl]pyridine (compound 37.4)

To a solution of cyclopropanol (3 g, 50.9 mmol) in DMF (40 mL) was added sodium hydride (60% dispersion in mineral oil, 3.4 g, 84.9 mmol) at 0° C. The resultant mixture was stirred at room temperature for 30 min, and then 4-bromo-2-(1-bromoethyl)pyridine (compound 37.3, 4.5 g, 17 mmol) was added. The reaction mixture was stirred at room temperature for another 30 min. The reaction was quenched with H₂O (20 mL), and extracted with EA (30 mL) for three times. The combined organic layer was washed with brine, dried over Na₂SO₄, filtered and concentrated in vacuo. The residue was purified by silica gel chromatography (PE/EA from 30/1 to 15/1) to afford compound 37.4 (2.9 g), which was used for next step directly without characterization.

### Step (d): Preparation of 2-[1-(cyclopropoxy)ethyl]-4-vinyl-pyridine (compound 37.5)

To the suspension of 4-bromo-2-[1-(cyclopropoxy)ethyl]pyridine (compound 37.4, 2.3 g, 9.5 mmol) and potassium vinyltrifluoroborate (3.2 g, 23.8 mmol) in isopropanol (45 mL) were added triethylamine (1.4 g, 2 mL, 14.3 mmol) and dichloro[1,1′-bis(diphenylphosphino)ferrocene]palladium(ii) dichloromethane adduct (193.9 mg, 237.5 μmol). After being stirred at 82° C. for 1.5 h under nitrogen, the reaction mixture was concentrated in vacuo. The residue was purified by silica gel chromatography (PE/EA from 30/1 to 20/1) to afford compound 37.5 (2.1 g). calc'd 190.1 [(M+H)⁺], measured 190.1 [(M+H)⁺].

### Step (e): Preparation of 2-[1-(cyclopropoxy)ethyl]pyridine-4-carbaldehyde (Compound 37.6)

To the suspension of 2-[1-(cyclopropoxy)ethyl]-4-vinyl-pyridine (compound 37.5, 2.1 g, 11.0 mmol) in 1,4-dioxane (80 mL)/water (40 mL) was added NaIO₄ (7.1 g, 33.0 mmol) and K₂OsO₄·2H₂O (202.5 mg, 549.5 μmol). The resultant mixture was stirred at 20° C. for 2 h. The reaction was quenched with sat. Na₂S₂O₃ and sat. NaHCO₃ respectively. The resultant mixture was stirred at room temperature for 1 h, and extracted with EA (150 mL) for three times. The organic layer was dried over Na₂SO₄ and concentrated in vacuo. The residue was purified by silica gel chromatography (PE/EA from 10/1 to 3/1) to afford compound 37.6 (1.4 g). calc'd 192.1 [(M+H)⁺], measured 224.0 [(M+MeOH+H)⁺].

### Step (f): Preparation of 1-[2-[1-(cyclopropoxy)ethyl]-4-pyridyl]-2,2-difluoro-ethanol (Compound 37.7)

CsF (222.4 mg, 1.5 mmol) was added to the mixture of 2-[1-(cyclopropoxy)ethyl]pyridine-4-carbaldehyde (compound 37.6, 1.4 g, 7.3 mmol) and (difluoromethyl)trimethylsilane (1.8 g, 14.6 mmol) in DMF (15 mL). The resultant mixture was stirred at 20° C. for 2 h. The reaction was quenched with H₂O (10 mL), and extracted with EA (20 mL) for three times The combined organic layer was washed with brine, dried over Na₂SO₄, filtered and concentrated. The residue was purified by silica gel chromatography (PE/EA from 10/1 to 1/1) to afford compound 37.7 (520 mg). calc'd 244.1 [(M+H)⁺], measured 244.0 [(M+H)⁺].

### Step (g): Preparation 1-[2-[1-(cyclopropoxy)ethyl]-4-pyridyl]-2,2-difluoro-ethanone (Compound 37.8)

To the mixture of 1-[2-[1-(cyclopropoxy)ethyl]-4-pyridyl]-2,2-difluoro-ethanol (compound 37.7, 520 mg, 2.1 mmol) in DCM (20 mL) was added DMP (1.8 g, 4.3 mmol) at 0° C. The resultant mixture was stirred at room temperature for 2 h. The reaction mixture was quenched with sat. Na₂S₂O₃ and sat. NaHCO₃ respectively, and extracted with DCM (50 mL) for three times. The combined organic layer was dried over Na₂SO₄, filtered and concentrated. The residue was purified by silica gel chromatography (PE/EA from 4/1 to 1/4) to afford compound 37.8 (530 mg). calc'd 242.1 [(M+H)⁺], measured 261.0 [(M+H₂O+H)⁺].

### Step (h): Preparation of (1S)-1-[2-[1-(cyclopropoxy)ethyl]-4-pyridyl]-2,2-difluoro-ethanol (Compound 37.9)

To the mixture of 1-[2-[1-(cyclopropoxy)ethyl]-4-pyridyl]-2,2-difluoro-ethanone (compound 37.8, 530 mg, 2.2 mmol) in DCM (4.0 mL) was added formic acid (2 g, 1.7 mL, 43.9 mmol), triethylamine (1.8 g, 2.4 mL, 17.6 mmol) and RuCl(p-cymene)[(R,R)-Ts-DPEN](69.9 mg, 109.9 μmol) at 0° C. The resultant mixture was stirred at room temperature for 1 h. The reaction mixture was quenched with sat. NaHCO₃ carefully, and extracted with DCM (20 mL) for three times. The combined organic layer was dried over Na₂SO₄, filtered and concentrated. The residue was purified by silica gel chromatography (PE/EA from 4/1 to 1/1) to afford compound 37.9 (520 mg). calc'd 244.1 [(M+H)⁺], measured 244.0 [(M+H)⁺].

### Step (i): Preparation of 3-[(1R)-1-[2-[1-(cyclopropoxy)ethyl]-4-pyridyl]-2,2-difluoro-ethoxy]-5-(2,4-ditert-butoxypyrimidin-5-yl)-1-methyl-pyrazolo[3,4-c]pyridazine (compound 37.10)

To the suspension of (1S)-1-[2-[1-(cyclopropoxy)ethyl]-4-pyridyl]-2,2-difluoro-ethanol (compound 37.9, 496.4 mg, 2 mmol) and 5-(2,4-ditert-butoxypyrimidin-5-yl)-1-methyl-pyrazolo[3,4-c]pyridazin-3-ol (intermediate B1, 400 mg, 1.1 mmol) and triphenylphosphane (597.8 mg, 2.2 mmol) in toluene (6 mL) was added DEAD (374.1 mg, 338.2 μL, 2.2 mmol) dropwise at 60° C. The resultant mixture was stirred at 60° C. for 30 min under nitrogen. The mixture was concentrated in vacuo. The residue was purified by silica gel chromatography (PE/EA from 1/1 to 3/2) to afford compound 37.10 (280 mg). calc'd 598.3 [(M+H)⁺], measured 598.6 [(M+H)⁺].

### Step (j): Preparation of 3-[(1R)-1-[2-[(1S)-1-(cyclopropoxy)ethyl]-4-pyridyl]-2,2-difluoro-ethoxy]-5-(2,4-ditert-butoxypyrimidin-5-yl)-1-methyl-pyrazolo[3,4-c]pyridazine and 3-[(1R)-1-[2-[(1R)-1-(cyclopropoxy)ethyl]-4-pyridyl]-2,2-difluoro-ethoxy]-5-(2,4-ditert-butoxypyrimidin-5-yl)-1-methyl-pyrazolo[3,4-c]pyridazine (compound 37.10A and compound 37.10B)

Compound 37.10 (280 mg) was resolved by SFC to give two single isomers: compound 37.10A (faster eluting, 107 mg) calc'd 598.3 [(M+H)⁺], measured 598.6.0 [(M+H)⁺]; and compound 37.10B (slower eluting, 100 mg) calc'd 598.3 [(M+H)⁺], measured 598.6 [(M+H)⁺]. SFC conditions: Column: IK 250×30 mm., 5 μm, mobile phase: A for CO₂ and B for Ethanol (0.1% NH₃H₂O), Gradient: B 25%, Flow rate: 80 mL/min, back pressure: 100 bar, Column temperature: 35° C.

### Step (k): Preparation of 5-[3-[(1R)-1-[2-[(1S)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione (Example 37A and Example 37B)

To the mixture of compound 37.10A (107 mg, 179 μmol) in methanol (1.7 mL) was added 2 M hydrogen chloride (447.6 μL, 895.2 μmol). The resultant mixture was stirred at room temperature for 1 h, then concentrated to afford a crude product, which was purified by pre-HPLC to afford Example 37A (49.5 mg). calc'd 486.2 [(M+H)⁺], measured 486.8 [(M+H)⁺]. ¹H NMR (400 MHz, DMSO-d₆) δ=11.56 (d, J=1.75 Hz, 1H), 11.45-11.51 (m, 1H), 8.74 (s, 1H), 8.62 (d, J=5.13 Hz, 1H), 8.38 (d, J=6.25 Hz, 1H), 7.71 (s, 1H), 7.61 (br d, J=5.13 Hz, 1H), 6.68 (td, J=53.78, 2.75 Hz, 1H), 6.36 (td, J=11.79, 2.44 Hz, 1H), 4.65 (q, J=6.59 Hz, 1H), 4.00 (s, 3H), 3.14 (tt, J=6.02, 3.05 Hz, 1H), 1.39 (d, J=6.63 Hz, 3H), 0.37-0.46 (m, 1H), 0.29-0.36 (m, 1H), 0.18-0.29 (m, 2H).

To the mixture of compound 37.10B (100 mg, 167.3 μmol) in methanol (1.6 mL) was added 2 M hydrogen chloride (418.3 μL, 836.6 μmol). The resultant mixture was stirred at room temperature for 1 h, then concentrated to afford a crude product, which was purified by pre-HPLC to afford Example 37B (56.2 mg). calc'd 486.2 [(M+H)⁺], measured 486.8 [(M+H)⁺]. ¹H NMR (400 MHz, DMSO-d₆) δ=11.55 (d, J=1.75 Hz, 1H), 11.47 (br d, J=5.00 Hz, 1H), 8.73 (s, 1H), 8.60 (d, J=5.13 Hz, 1H), 8.37 (d, J=6.25 Hz, 1H), 7.74 (s, 1H), 7.59 (br d, J=4.88 Hz, 1H), 6.67 (td, J=53.41, 2.75 Hz, 1H), 6.30-6.41 (m, 1H), 4.66 (q, J=6.55 Hz, 1H), 4.00 (s, 3H), 3.23 (dt, J=6.03, 3.05 Hz, 1H), 1.37 (d, J=6.63 Hz, 3H), 0.49-0.57 (m, 1H), 0.30-0.46 (m, 3H).

### Example 38

### 5-[3-[(1R)-1-[2-(3,3-difluorocyclobutyl)-4-pyridyl]-2,2-difluoro-ethoxy]-1-methyl-pyrazolo[3,4-c]pyridazin-5-yl]-1H-pyrimidine-2,4-dione

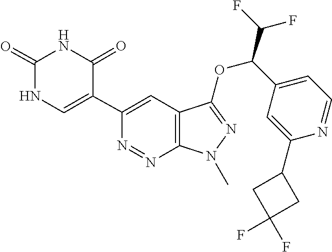 [SMILES: Cn1nc(O[C@H](c2ccnc(C3CC(F)(F)C3)c2)C(F)F)c2cc(-c3c[nH]c(=O)[nH]c3=O)nnc21]

The titled compound was synthesized according to the following scheme:

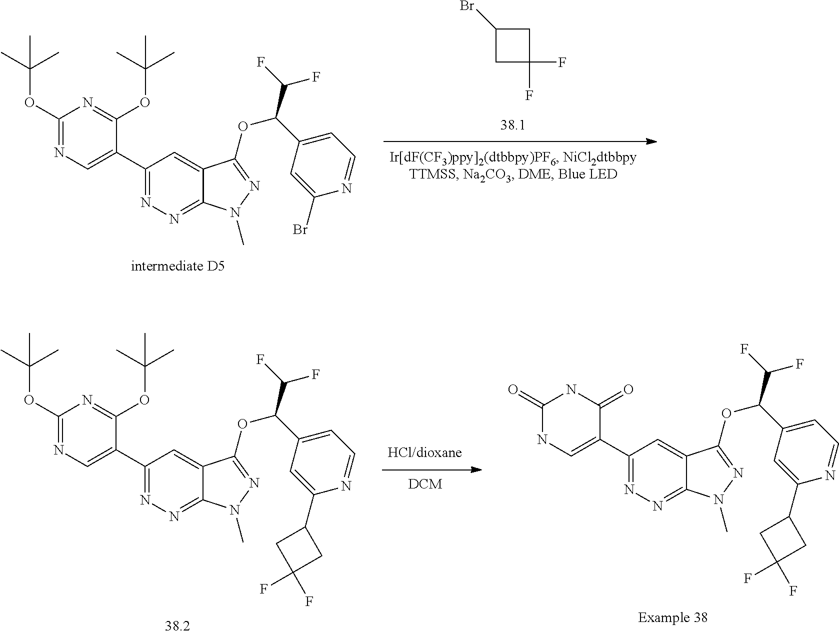

### Step (a): Preparation of 5-(2,4-ditert-butoxypyrimidin-5-yl)-3-[(1R)-1-[2-(3,3-difluorocyclobutyl)-4-pyridyl]-2,2-difluoro-ethoxy]-1-methyl-pyrazolo[3,4-c]pyridazine (Compound 38.2)

A mixture of 3-[(1R)-1-(2-bromo-4-pyridyl)-2,2-difluoro-ethoxy]-5-(2,4-ditert-butoxypyrimidin-5-yl)-1-methyl-pyrazolo[3,4-c]pyridazine (intermediate D5, 500 mg, 0.84 mmol), [4,4′-bis(1,1-dimethylethyl)-2,2′-bipyridine]nickel dichloride (67.2 mg, 0.17 mmol), 3-bromo-1,1-difluoro-cyclobutane (compound 38.1, 433 mg, 2.53 mmol), tris(trimethylsilyl)silane (0.31 mL, 1.01 mmol), Ir[dF(CF₃)ppy]₂ (dtbbpy)PF₆ (47.4 mg, 0.04 mmol) and sodium carbonate (268 mg, 2.53 mmol) in ethylene glycol dimethyl ether (5 mL) was irradiated with blue LED light for 3 h at 25° C. under nitrogen atmosphere. The reaction mixture was quenched with water, and extracted with EA (30 mL) for three times. The combined organic layer was washed with brine, dried over Na₂SO₄, filtered and concentrated. The residue was purified by flash chromatography (silica gel, 20 g, 0 to 100% EA in PE) to afford the desired product compound 38.2 (146 mg). MS: calc'd 604.3 [(M+H)⁺]; measured 604.3 [(M+H)⁺].

### Step (b): Preparation of 5-[3-[(1R)-1-[2-(3,3-difluorocyclobutyl)-4-pyridyl]-2,2-difluoro-ethoxy]-1-methyl-pyrazolo[3,4-c]pyridazin-5-yl]-1H-pyrimidine-2,4-dione (Example 38)

To a solution of 5-(2,4-ditert-butoxypyrimidin-5-yl)-3-[(1R)-1-[2-(3,3-difluorocyclobutyl)-4-pyridyl]-2,2-difluoro-ethoxy]-1-methyl-pyrazolo[3,4-c]pyridazine (compound 38.2, 140 mg, 0.23 mmol) in DCM (5 mL) was added 4N HCl/dioxane (0.5 mL). The resultant mixture was stirred at room temperature for 2 h, then concentrated to afford a crude product, which was purified by prep-HPLC to afford Example 186 (37.6 mg). MS: calc'd 492.1 [(M+H)⁺]; measured 492.2 [(M+H)⁺]. ¹H NMR (400 MHz, DMSO-d₆) δ 11.26 (br s, 2H), 8.72 (d, J=1.6 Hz, 1H), 8.65 (d, J=5.1 Hz, 1H), 8.39 (s, 1H), 7.59 (s, 1H), 7.50 (dd, J=5.1, 1.6 Hz, 1H), 6.64 (td, J=53.8, 2.9 Hz, 1H), 6.28 (td, J=12.4, 11.9, 2.9 Hz, 1H), 4.01 (s, 3H), 3.56 (qt, J=8.6, 4.2 Hz, 1H), 3.02-2.76 (m, 4H).

### Example 39A and Example 39B

### 5-[3-[(1R)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione and 5-[3-[(1S)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione

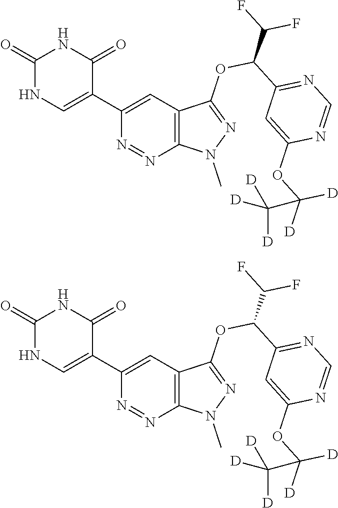

Example 39A and Example 39B was prepared in analogy to Example 35A and Example 35B, by replacing 2,2,2-trifluoroethanol with 1,1,2,2,2-pentadeuterioethanol in step (b).

Example 39A MS: calc'd 452.2 [(M+H)⁺]; measured 452.2 [(M+H)⁺]. ¹H NMR (400 MHz, DMSO-d₆) δ=11.46 (br s, 2H), 8.83 (d, J=1.2 Hz, 1H), 8.72 (d, J=1.1 Hz, 1H), 8.38 (s, 1H), 7.15 (s, 1H), 6.71 (td, J=53.5, 2.5 Hz, 1H), 6.26-6.09 (m, 1H), 4.03 (s, 3H).

Example 39B MS: calc'd 452.2 [(M+H)⁺]; measured 452.2 [(M+H)⁺]. ¹H NMR (400 MHz, DMSO-d₆) δ=11.46 (br s, 2H), 8.83 (d, J=1.2 Hz, 1H), 8.72 (d, J=1.1 Hz, 1H), 8.38 (s, 1H), 7.15 (s, 1H), 6.71 (td, J=53.5, 2.5 Hz, 1H), 6.24-6.10 (m, 1H), 4.03 (s, 3H).

### Example 40

### 5-[1-methyl-3-[(1R)-2,2-difluoro-1-[2-[(trans)-4-methyltetrahydrofuran-3-yl]oxy-4-pyridyl]ethoxy]pyrazolo[3,4-c]pyridazin-5-yl]-1H-pyrimidine-2,4-dione

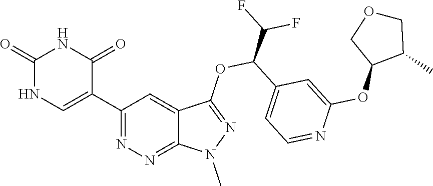 [SMILES: C[C@H]1COC[C@@H]1Oc1cc([C@@H](Oc2nn(C)c3nnc(-c4c[nH]c(=O)[nH]c4=O)cc23)C(F)F)ccn1]

Example 40 was prepared in analogy to Example 28, by replacing 2-methoxyethanol with (trans)-4-methyltetrahydrofuran-3-ol in step (a). MS: calc'd 502.2 [(M+H)⁺]; measured 502.1 [(M+H)⁺]. ¹H NMR (400 MHz, DMSO-d₆) δ=11.48 (d, J=1.8 Hz, 1H), 11.38 (dd, J=1.6, 6.0 Hz, 1H), 8.64 (s, 1H), 8.30 (d, J=6.3 Hz, 1H), 8.13 (d, J=5.4 Hz, 1H), 7.13 (dd, J=0.9, 5.3 Hz, 1H), 6.96 (s, 1H), 6.52 (dt, J=2.8, 54.3 Hz, 1H), 6.24-6.12 (m, 1H), 5.05-4.92 (m, 1H), 3.95 (s, 3H), 3.94-3.85 (m, 2H), 3.64 (ddd, J=1.9, 5.3, 10.4 Hz, 1H), 2.34-2.18 (m, 1H), 1.28-1.14 (m, 1H), 0.99 (dd, J=2.6, 7.1 Hz, 3H).

### Example 41

### 5-[3-[(1R)-2,2-difluoro-1-[2-[(1-methoxycyclopropyl)methoxy]-4-pyridyl]ethoxy]-1-methyl-pyrazolo[3,4-c]pyridazin-5-yl]-1H-pyrimidine-2,4-dione

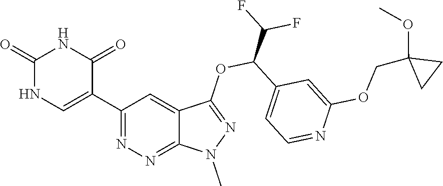 [SMILES: COC1(COc2cc([C@@H](Oc3nn(C)c4nnc(-c5c[nH]c(=O)[nH]c5=O)cc34)C(F)F)ccn2)CC1]

Example 41 was prepared in analogy to Example 28, by replacing 2-methoxyethanol with (1-methoxycyclopropyl)methanol in step (a). MS: calc'd 502.2 [(M+H)⁺]; measured 502.1 [(M+H)⁺]. ¹H NMR (400 MHz, DMSO-d₆) δ=11.55 (d, J=1.5 Hz, 1H), 11.45 (dd, J=1.5, 6.1 Hz, 1H), 8.72 (s, 1H), 8.37 (d, J=6.1 Hz, 1H), 8.18 (d, J=5.2 Hz, 1H), 7.19 (dd, J=1.2, 5.3 Hz, 1H), 7.06 (s, 1H), 6.63 (dt, J=3.2, 53.9 Hz, 1H), 6.30-6.21 (m, 1H), 4.43-4.33 (m, 2H), 4.02 (s, 3H), 3.24 (s, 3H), 0.79 (d, J=1.8 Hz, 2H), 0.70-0.64 (m, 2H).

### Example 42A and Example 42B

### 5-[3-[(1R)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione and 5-[3-[(1S)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione

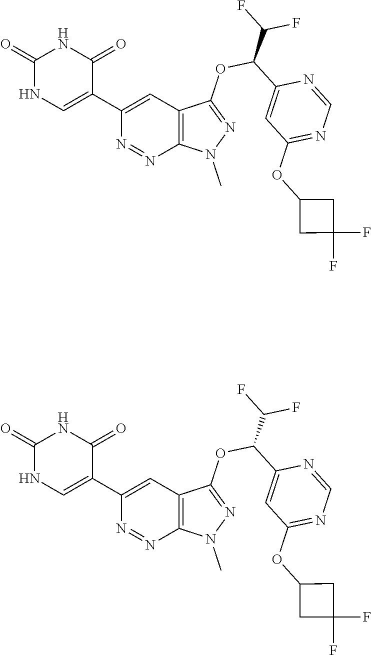 [SMILES: Cn1nc(O[C@H](c2cc(OC3CC(F)(F)C3)ncn2)C(F)F)c2cc(-c3c[nH]c(=O)[nH]c3=O)nnc21 ; Cn1nc(O[C@@H](c2cc(OC3CC(F)(F)C3)ncn2)C(F)F)c2cc(-c3c[nH]c(=O)[nH]c3=O)nnc21]

The titled compound was synthesized according to the following scheme:

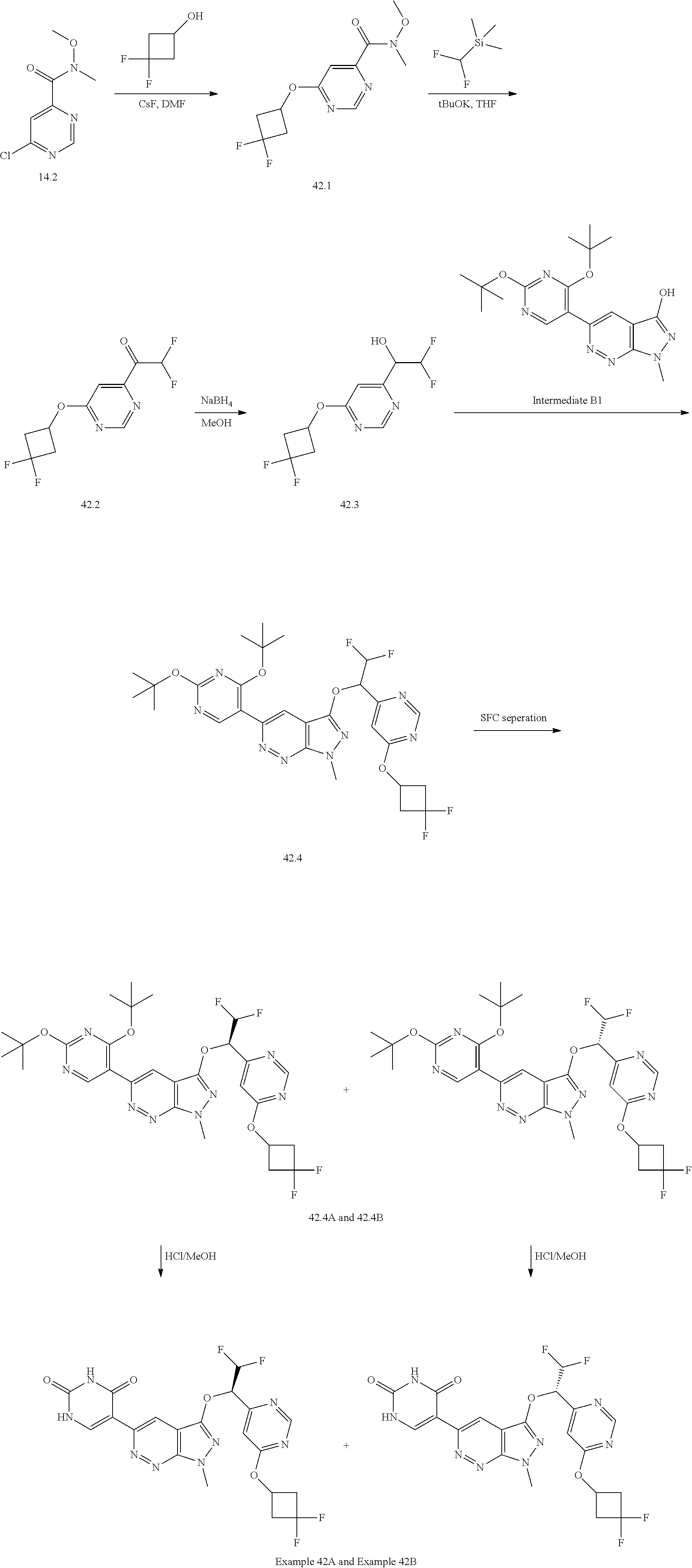

Step (a): Preparation of 6-(3,3-difluorocyclobutoxy)-N-methoxy-N-methyl-pyrimidine-4-carboxamide (compound 42.1).

To a solution of 3,3-difluorocyclobutanol (3.22 g, 29.76 mmol) and 6-chloro-N-methoxy-N-methyl-pyrimidine-4-carboxamide (compound 14.2, 3.0 g, 14.88 mmol) in DMF (30 mL) was added cesium fluoride (6.78 g, 44.64 mmol), the resultant mixture was stirred at 80° C. for 5 hr.

After being cooled to room temperature, the reaction mixture was quenched with water (300 mL), and extracted with EA (100 mL) for three times. The combined organic layer was dried over Na₂SO₄, filtered and concentrated. The residue was purified by flash chromatography (silica gel, 0 to 50% EA in PE) to afford compound 42.1 (3.3 g). MS: calc'd 274.1 [(M+H)⁺]; measured 274.0 [(M+H)⁺].

### Step (b): Preparation of 1-[6-(3,3-difluorocyclobutoxy)pyrimidin-4-yl]-2,2-difluoro-ethanone (Compound 42.2)

To a solution of (difluoromethyl)trimethylsilane (4.09 g, 32.94 mmol) and 6-(3,3-difluorocyclobutoxy)-N-methoxy-N-methyl-pyrimidine-4-carboxamide (compound 42.1, 4500 mg, 16.47 mmol) in THF (50 mL) was added tBuOK (2.4 g, 21.41 mmol) at 0° C. The resultant mixture was stirred at 0° C. for 60 min. The reaction mixture was diluted with water (100 mL), extracted with EA (80 mL) twice. The combined organic layer was washed with brine, dried over Na₂SO₄, filtered and concentrated. The residue was purified by flash chromatography (silica gel, 80 g, 0 to 90% EA in PE) to afford compound 42.2 (2700 mg). MS: calc'd 274.1 [(M+H)⁺]; measured 282.1 [(M+H)⁺].

### Step (c): Preparation of 5-[3-[(1R)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione and 5-[3-[(1S)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione (Example 42A and Example 42B)

Example 42A and Example 42B was prepared in analogy to Example 35A and Example 35B, by replacing 2,2-difluoro-1-[6-(2,2,2-trifluoroethoxy)pyrimidin-4-yl]ethanol (compound 35.3) with 1-[6-(3,3-difluorocyclobutoxy)pyrimidin-4-yl]-2,2-difluoro-ethanone (compound 42.2) in step (d).

Example 42A MS: calc'd 509.1 [(M+H)⁺]; measured 509.1 [(M+H)⁺]. ¹H NMR (400 MHz, DMSO-d₆) δ=11.55 (d, J=1.8 Hz, 1H), 11.46 (dd, J=1.5, 6.1 Hz, 1H), 8.87 (d, J=1.1 Hz, 1H), 8.72 (s, 1H), 8.38 (d, J=6.1 Hz, 1H), 7.25 (s, 1H), 6.72 (dt, J=2.5, 53.3 Hz, 1H), 6.21 (ddd, J=2.9, 8.7, 14.8 Hz, 1H), 5.27-5.14 (m, 1H), 4.02 (s, 3H), 3.26-3.07 (m, 2H), 2.89-2.68 (m, 2H).

Example 42B MS: calc'd 509.1 [(M+H)⁺]; measured 509.1 [(M+H)⁺]. ¹H NMR (400 MHz, DMSO-d₆) δ=11.55 (d, J=1.5 Hz, 1H), 11.49-11.41 (m, 1H), 8.87 (d, J=1.0 Hz, 1H), 8.72 (s, 1H), 8.38 (d, J=6.1 Hz, 1H), 7.25 (s, 1H), 6.72 (dt, J=2.9, 53.2 Hz, 1H), 6.21 (ddd, J=2.6, 8.7, 14.9 Hz, 1H), 5.27-5.14 (m, 1H), 4.02 (s, 3H), 3.26-3.04 (m, 2H), 2.87-2.69 (m, 2H).

### Reference Compound:

Compound G531 disclosed in WO2021222522 as example 531 was chosen as the reference compound for this application.

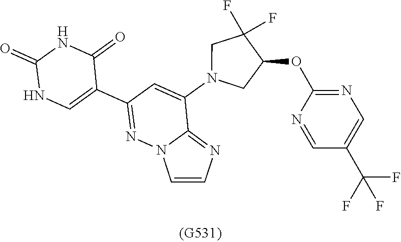 [SMILES: O=c1[nH]cc(-c2cc(N3C[C@H](Oc4ncc(C(F)(F)F)cn4)C(F)(F)C3)c3nccn3n2)c(=O)[nH]1]

### BIOLOGICAL EXAMPLE

### Example 43: Human CD73 Biochemical Assay

The purpose of this assay is to identify and characterize inhibitors of human CD73 enzymatic activity. Compound serial dilution (1:3) was prepared with Echo 555 liquid handler (Labcyte) into the corresponding wells of a 384-well plate. 10 μL of enzyme working solution (containing 0.05 nM recombinant CD73 protein, 10 mM Tris pH 7.5, 100 mM NaCl, 0.01% BSA, 0.2 mM Octyl glucoside and 1 mM NaH₂PO₄) was added to the assay plate and incubated with compounds for 15 minutes at room temperature. After adding 15 μL of AMP working solution (Containing 10 mM Tris pH 7.5, 100 mM NaCl, 0.01% BSA, 0.2 mM Octyl glucoside, with 5.56 μM of AMP final concentration) and incubate for 10 min at room temperature. Stop the reaction by adding 75 μL of stop solution (5% TCA in H₂O containing 250 nM 13C5-adenosine) to each well for 10 min incubation. After centrifugation, 75 μL of the mixture was transferred to a new 384-well plate for LC/MS analysis.

Samples from the 384-well plates, were loaded onto an autosampler deck, then injected with ADDA-LC-MS/MS. The aqueous mobile phase is 0.1% formic acid in water. The organic mobile phase is 0.1% formic acid in acetonitrile. Flow rate is maintained at 0.8 mL/minute using Shimadzu pumps. The column is ACE 5 Phenyl, 50×2.1 mm. The analysis was performed on a SCIEX triple quadrupole mass spectrometer operating in positive ion mode. The effluent from the HPLC column was directly introduced into the electrospray ionization (ESI). Multiple reaction monitoring (MRM) is used to determine analyte and internal standard (IS) responses. The MRM for adenosine is 268.1/136.1, for 13C5-Adenosine (IS) is 273.2/136.2. The data is calculated using the peak area ratio (PAR) semi-quantitative method.

TABLE 1
Enzymatic IC₅₀ values of the compounds
of this invention against human CD73

| Example No | IC₅₀ (nM) |
|---|---|
| 1 | 0.08 |
| 2 | 0.07 |
| 3 | 0.17 |
| 4 | 0.07 |
| 7 | 0.08 |
| 8A | 0.07 |
| 9A | 0.11 |
| 10 | 0.06 |
| 11B | 0.05 |
| 12 | 0.02 |
| 13A | 0.02 |
| 14A | 0.09 |
| 15 | 0.07 |
| 16 | 0.04 |
| 20 | 0.06 |
| 21 | 0.09 |
| 23 | 0.08 |
| 24 | 0.05 |
| 26 | 0.04 |
| 27 | 0.04 |
| 28 | 0.05 |
| 30 | 0.05 |
| 31 | 0.05 |
| 32 | 0.05 |
| 34 | 0.07 |
| 35A | 0.06 |
| 36A | 0.08 |
| 37A | 0.13 |
| 37B | 0.09 |
| 38 | 0.06 |
| 40 | 0.05 |
| 42A | 0.06 |

### Example 44: CD73 Cellular Assay

Compound serial dilution (1:3) was prepared with Echo 555 liquid handler (Labcyte) into the corresponding wells of a 384-well plate. 40 μL of MDA-MB-231 cells (ATCC, HTB-26, breast cancer, final concentration at 20,000 cells/mL) suspended in assay buffer (20 mM HEPES pH 7.4, 137 mM NaCl, 5.4 mM KCl, 1.3 mM CaCl₂, 4.2 mM NaHCO₃, 1 mg/mL glucose) were added into the corresponding wells of the plate. After incubating with compounds for 30 minutes, 40 μL of AMP working solution (200 μM AMP in assay buffer) was added into each well in the assay plate. The assay plate was then incubated in a 5% CO₂ incubator (Thermo Fisher Scientific) at 37° C. for 45 min. After the reaction was completed, 50 μL of the supernatants was collected and transferred into a new 384 well plate. 10 μL of Malachite A was added to each well in the assay plate and incubated for 10 minutes. 10 μL of Malachite B was then added to each corresponding well of the plate and incubated for 30 minutes. Finally the absorbance value was read on the Envision plate reader at 620 nM. Calculation percentage inhibition by using equation {% inhibition=100×[1−(X−MIN)/(MAX−MIN)]} where X equals to the well signal, Max equals signal of neutral control and MIN equals signal of inhibitor control.

TABLE 2
Cellular IC₅₀ values of the compounds
of this invention against CD73

| Example No | IC₅₀ (nM) |
|---|---|
| G351 | 3.53 |
| 1 | 0.70 |
| 2 | 0.21 |
| 3 | 0.91 |
| 4 | 0.49 |
| 5 | 0.52 |
| 6 | 0.43 |
| 7 | 0.59 |
| 8A | 0.66 |
| 9A | 0.73 |
| 10 | 0.55 |
| 11B | 0.72 |
| 12 | 0.67 |
| 13A | 0.97 |
| 14A | 1.04 |
| 15 | 0.88 |
| 16 | 0.54 |
| 17 | 0.39 |
| 18 | 0.54 |
| 19 | 0.59 |
| 20 | 0.49 |
| 21 | 0.73 |
| 22 | 0.43 |
| 23 | 0.74 |
| 24 | 0.44 |
| 25 | 1.29 |
| 26 | 0.27 |
| 27 | 0.48 |
| 28 | 0.63 |
| 29 | 0.51 |
| 30 | 0.60 |
| 31 | 0.55 |
| 32 | 0.38 |
| 33 | 0.63 |
| 34 | 0.42 |
| 35A | 0.59 |
| 36A | 0.62 |
| 37A | 0.87 |
| 37B | 0.84 |
| 38 | 0.36 |
| 39A | 0.65 |
| 40 | 0.57 |
| 41 | 0.73 |
| 42A | 0.76 |

### Example 45: T Cell Proliferation Assay

The purpose of this assay is to characterize the potency of inhibitors of CD73 in rescuing adenosine-mediated inhibition of T cell proliferation. CD4+ or CD8+ T cells were isolated from peripheral blood mononuclear cells (PBMCs, HemaCare) by immunomagnetic negative selection using EasySep™ Isolation Kit (STEMCELL Technologies) following the supplier's protocol. Pan T cells were pelleted by centrifugation at 300 gravitational force (g) for 5 minutes at room temperature and re-suspended in PBS. CellTrace™ Violet staining solution (Invitrogen) was added at 1:5,000 and incubated at 37° C. for 20 minutes, protected from light. Complete culture medium [RPMI-1640 (Gibco), 10% Fetal Bovine Serum (Gibco), 2 mM GlutaMAX (Gibco) and 1 mM Sodium Pyruvate (Gibco), 100 U/mL Penicillin-Streptomycin (Gibco) and MEM non-essential amino acids (NEAA) cell culture supplement (1:100, Gibco)] was then added, mixed, and incubated at 37° C. for 5 minutes. Cells were then pelleted by centrifugation at 300 g for 5 minutes at room temperature and re-suspended in fresh, pre-warmed complete culture medium. 50 μL of cells were seeded per well in 96 well u-bottom plates. 50 μL of CD3/CD28 beads-containing medium and 50 μL of media containing compounds were added into cells. 50 μL of media containing AMP and EHNA hydrochloride (Sigma-Aldrich) was added into cells at 200 μM and 5 μM final concentration, respectively. Cells were incubated for 72 hours at 37° C. in a 5% CO₂ incubator. 200 μL of PBS was then added to each well and cells were centrifuged at 300 g, 4° C. for 10 minutes. The supernatant was discarded. 50 μL of Human TruStain FcX™ (Fc Receptor Blocking Solution, BioLegend) diluted 1:100 in PBS was added to each well, mixed gently and incubated for 20 minutes at 4° C. 50 μL of staining solution (BioLegend) was added to each well, mixed gently and incubated at 4° C. for 30 minutes. Cells were centrifuged at 300 g, 4° C. for 10 minutes and the supernatant was discarded. The cell pellets were washed with 250 μL of cell staining buffer and centrifuged at 300 g, 4° C. for 10 minutes. The supernatant was discarded and cells were re-suspended in 60 μL of cell staining buffer and analyzed on a flow cytometer.

TABLE 3
Rescue of proliferation of CD4+ and CD8+ T cells
under high AMP condition by CD73 inhibitors

| | CD4+ T cell proliferation | CD8+ T cell proliferation |
|---|---|---|
| Example No | EC₅₀ (nM) | EC₅₀ (nM) |
| G351 | 8.8 | 8.2 |
| 2 | 0.18 | 0.17 |
| 4 | 0.18 | 0.22 |
| 10 | 0.14 | 0.08 |
| 11B | 0.24 | 0.29 |
| 12 | 0.15 | 0.12 |
| 24 | 0.19 | 0.23 |
| 26 | 0.08 | 0.09 |
| 28 | 0.33 | 0.56 |
| 34 | 0.10 | 0.11 |
| 35A | 0.30 | 0.32 |
| 38 | 0.06 | 0.07 |
| 42A | 0.22 | 0.18 |

### Example 46: T Cell Cytokine Release Function Assay

The purpose of this assay is to characterize the potency of inhibitors of CD73 in rescuing adenosine-mediated inhibition of T cell cytokine release function. Pan T cells were isolated from peripheral blood mononuclear cells (PBMCs, HemaCare) by immunomagnetic negative selection using EasySep™ Isolation Kit (STEMCELL Technologies) following the supplier's protocol. T cells were then pelleted the cells by centrifugation at 300 g for 5 minutes at room temperature and re-suspended in fresh, pre-warmed complete culture medium. 50 μL of cells was seeded per well in 96 well u-bottom plates. 50 μL of CD3/CD28 beads-containing medium and 50 μL of media containing compounds were added into cells. 50 μL of media containing AMP and EHNA hydrochloride (Sigma-Aldrich) was added into cells at 200 μM and 5 μM final concentration, respectively. Cells were incubated for up to 72 hours at 37° C. in a 5% CO₂ incubator. 50 μL of supernatant was collected to determine levels of IFN gamma using Luminex assay (Thermo Fischer Scientific).

1. A compound of formula (I), 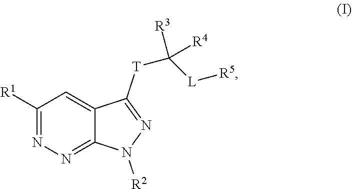
wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (C₁₋₆alkoxyC₃₋₇cycloalkyl)C₁₋₆alkoxy, (C₁₋₆alkyltetrahydrofuranyl)oxy, (cyanoC₃₋₇cycloalkyl)C₁₋₆alkoxy, (haloC₁₋₆alkoxy)C₁₋₆alkyl, (haloC₁₋₆alkylC₃₋₇cycloalkyl)C₁₋₆alkoxy, (haloC₃₋₇cycloalkyl)amino, (haloC₃₋₇cycloalkyl)C₁₋₆alkoxy, (halopyrrolidinyl)C₁₋₆alkyl, C₁₋₆alkoxy, C₁₋₆alkoxyC₁₋₆alkoxy, C₃₋₇cycloalkoxyC₁₋₆alkyl, cyanoC₁₋₆alkoxy, deuterioC₁₋₆alkoxy, deuteriohaloC₁₋₆alkoxy, haloC₁₋₆alkoxy, haloC₁₋₆alkyl, haloC₁₋₆alkylamino, haloC₃₋₇cycloalkoxy, haloC₃₋₇cycloalkyl or halopiperidyl;
T is O;
L is 1,4-benzoxazinylene, indazolylene, pyrimidinylene, phenylene being unsubstituted or substituted by halogen, or pyridylene being unsubstituted or substituted by halogen;
or a pharmaceutically acceptable salt thereof.

2. A compound according to claim 1 having the structure of formula (I-1): 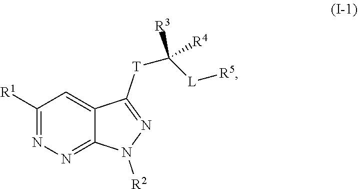
wherein
R¹ is 2,4-dioxo-1H-pyrimidinyl or 3,5-dioxo-2H-1,2,4-triazinyl;
R² is C₁₋₆alkyl or deuterioC₁₋₆alkyl;
R³ is C₁₋₆alkyl or haloC₁₋₆alkyl;
R⁴ is H;
R⁵ is (C₁₋₆alkoxyC₃₋₇cycloalkyl)C₁₋₆alkoxy, (C₁₋₆alkyltetrahydrofuranyl)oxy, (cyanoC₃₋₇cycloalkyl)C₁₋₆alkoxy, (haloC₁₋₆alkoxy)C₁₋₆alkyl, (haloC₁₋₆alkylC₃₋₇cycloalkyl)C₁₋₆alkoxy, (haloC₃₋₇cycloalkyl)amino, (haloC₃₋₇cycloalkyl)C₁₋₆alkoxy, (halopyrrolidinyl)C₁₋₆alkyl, C₁₋₆alkoxy, C₁₋₆alkoxyC₁₋₆alkoxy, C₃₋₇cycloalkoxyC₁₋₆alkyl, cyanoC₁₋₆alkoxy, deuterioC₁₋₆alkoxy, deuteriohaloC₁₋₆alkoxy, haloC₁₋₆alkoxy, haloC₁₋₆alkyl, haloC₁₋₆alkylamino, haloC₃₋₇cycloalkoxy, haloC₃₋₇cycloalkyl or halopiperidyl;
T is O;
L is 1,4-benzoxazinylene, indazolylene, pyrimidinylene, phenylene being unsubstituted or substituted by halogen, or pyridylene being unsubstituted or substituted by halogen;
or a pharmaceutically acceptable salt thereof.

3. A compound according to claim 1, wherein R¹ is

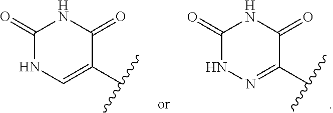 [SMILES: *c1c[nH]c(=O)[nH]c1=O ; *c1n[nH]c(=O)[nH]c1=O]

4. A compound according to claim 1, wherein R² is C₁₋₆alkyl.

5. A compound according to claim 1, wherein R² is methyl.

6. A compound according to any claim 1, wherein R³ is haloC₁₋₆alkyl.

7. A compound according to claim 1, wherein R³ is difluoromethyl.

8. A compound according to claim 1, wherein L is pyrimidinylene, or pyridylene being unsubstituted or substituted by halogen.

9. A compound according to claim 1, wherein L is pyrimidinylene, or pyridylene being unsubstituted or substituted by fluoro.

10. A compound according to claim 1, wherein R⁵ is (haloC₁₋₆alkoxy)C₁₋₆alkyl, (halopyrrolidinyl)C₁₋₆alkyl, C₃₋₇cycloalkoxyC₁₋₆alkyl, haloC₁₋₆alkoxy, haloC₁₋₆alkylamino, haloC₃₋₇cycloalkoxy or haloC₃₋₇cycloalkyl.

11. A compound according to claim 1, wherein R⁵ is (3,3-difluoropyrrolidin-1-yl)methyl, [2,2,2-trifluoro-1-methyl-ethyl]amino, 2,2,2-trifluoroethoxy, 2,2,2-trifluoroethoxymethyl, 2,2-difluoroethoxy, 3,3-difluorocyclobutoxy, 3,3-difluorocyclobutyl or cyclopropoxymethyl.

12. A compound according to claim 1, wherein:
R¹ is 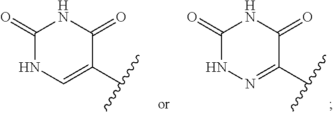 [SMILES: *c1c[nH]c(=O)[nH]c1=O ; *c1n[nH]c(=O)[nH]c1=O]
R² is C₁₋₆alkyl;
R³ is haloC₁₋₆alkyl;
R⁴ is H;
R⁵ is (haloC₁₋₆alkoxy)C₁₋₆alkyl, (halopyrrolidinyl)C₁₋₆alkyl, C₃₋₇cycloalkoxyC₁₋₆alkyl, haloC₁₋₆alkoxy, haloC₁₋₆alkylamino, haloC₃₋₇cycloalkoxy or haloC₃₋₇cycloalkyl;
T is O;
L is pyrimidinylene, or pyridylene being unsubstituted or substituted by halogen;
or a pharmaceutically acceptable salt thereof.

13. A compound according to claim 12, wherein
R¹ is 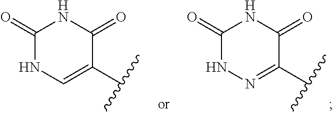 [SMILES: *c1c[nH]c(=O)[nH]c1=O ; *c1n[nH]c(=O)[nH]c1=O]
R² is methyl;
R³ is difluoromethyl;
R⁴ is H;
R⁵ is (3,3-difluoropyrrolidin-1-yl)methyl, [2,2,2-trifluoro-1-methyl-ethyl]amino, 2,2,2-trifluoroethoxy, 2,2,2-trifluoroethoxymethyl, 2,2-difluoroethoxy, 3,3-difluorocyclobutoxy, 3,3-difluorocyclobutyl or cyclopropoxymethyl;
T is O;
L is pyrimidinylene, or pyridylene being unsubstituted or substituted by fluoro;
or a pharmaceutically acceptable salt thereof.

14. A compound selected from:
5-[3-[(1R)-2,2-difluoro-1-[3-(2,2,2-trifluoroethoxy)phenyl]ethoxy]-1-methyl-pyrazolo[3,4-c]pyridazin-5-yl]-1H-pyrimidine-2,4-dione;
5-[3-[(1R)-1-[2-(cyclopropoxymethyl)-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[4-fluoro-3-(2,2,2-trifluoroethoxy)phenyl]ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1R)-1-[4-(1,1-dideuterio-2,2,2-trifluoro-ethoxy)-2-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethoxy)-2-pyridyl]ethoxy]-1-(trideuteriomethyl)pyrazolo[3,4-c]pyridazin-5-yl]-1H-pyrimidine-2,4-dione;
5-[3-[(1R)-1-[4-(2,2-difluoroethoxy)-2-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione;
5-[3-[(1S)-2,2-difluoro-1-[5-(2,2,2-trifluoroethoxy)-3-pyridyl]ethoxy]-1-methyl-pyrazolo[3,4-c]pyridazin-5-yl]-1H-pyrimidine-2,4-dione;
5-[3-[(1R)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1S)-2,2-difluoro-1-[5-fluoro-4-(2,2,2-trifluoroethoxy)-2-pyridyl]ethoxy]-1-methyl-pyrazolo[3,4-c]pyridazin-5-yl]-1H-pyrimidine-2,4-dione;
5-[3-[(1R)-2,2-difluoro-1-[2-(2,2,2-trifluoroethoxy)-4-pyridyl]ethoxy]-1-methyl-pyrazolo[3,4-c]pyridazin-5-yl]-1H-pyrimidine-2,4-dione;
5-[3-[(1S)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-(2,2-difluoroethoxy)-4-pyridyl]-2,2-difluoro-ethoxy]-1-methyl-pyrazolo[3,4-c]pyridazin-5-yl]-1H-pyrimidine-2,4-dione;
6-[3-[(1R)-2,2-difluoro-1-[2-(2,2,2-trifluoroethoxy)-4-pyridyl]ethoxy]-1-methyl-pyrazolo[3,4-c]pyridazin-5-yl]-2H-1,2,4-triazine-3,5-dione;
6-[3-[(1R)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione;
6-[3-[(1S)-1-[2-(2,2-difluoroethoxy)-4-pyridyl]-2,2-difluoro-ethoxy]-1-methyl-pyrazolo[3,4-c]pyridazin-5-yl]-2H-1,2,4-triazine-3,5-dione;
5-[1-methyl-3-[(1S)-1-[6-(2,2,2-trifluoroethoxy)pyrimidin-4-yl]ethoxy]pyrazolo[3,4-c]pyridazin-5-yl]-1H-pyrimidine-2,4-dione;
5-[1-methyl-3-[(1R)-1-[6-(2,2,2-trifluoroethoxy)pyrimidin-4-yl]ethoxy]pyrazolo[3,4-c]pyridazin-5-yl]-1H-pyrimidine-2,4-dione;
5-[3-[(1R)-2,2-difluoro-1-[4-fluoro-3-(2-methoxyethoxy)phenyl]ethoxy]-1-methyl-pyrazolo[3,4-c]pyridazin-5-yl]-1H-pyrimidine-2,4-dione;
5-[3-[(1R)-1-[2-(2,2-difluoropropoxy)-4-pyridyl]-2,2-difluoro-ethoxy]-1-methyl-pyrazolo[3,4-c]pyridazin-5-yl]-1H-pyrimidine-2,4-dione;
5-[1-methyl-3-[(1R)-2,2-difluoro-1-[4-(2,2,2-trifluoro-1-methyl-ethoxy)-2-pyridyl]ethoxy]pyrazolo[3,4-c]pyridazin-5-yl]-1H-pyrimidine-2,4-dione;
5-[1-methyl-3-[(1R)-1-[4-[(2,2-difluorocyclopropyl)methoxy]-2-pyridyl]-2,2-difluoro-ethoxy]pyrazolo[3,4-c]pyridazin-5-yl]-1H-pyrimidine-2,4-dione;
5-[3-[(1R)-2,2-difluoro-1-[4-[(1-fluorocyclopropyl)methoxy]-2-pyridyl]ethoxy]-1-methyl-pyrazolo[3,4-c]pyridazin-5-yl]-1H-pyrimidine-2,4-dione;
5-[3-[(1R)-1-(4-ethoxy-2-pyridyl)-2,2-difluoro-ethoxy]-1-methyl-pyrazolo[3,4-c]pyridazin-5-yl]-1H-pyrimidine-2,4-dione;
2-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxy]acetonitrile;
1-[[2-[(1R)-1-[5-(2,4-dioxo-1H-pyrimidin-5-yl)-1-methyl-pyrazolo[3,4-c]pyridazin-3-yl]oxy-2,2-difluoro-ethyl]-4-pyridyl]oxymethyl]cyclopropanecarbonitrile;
5-[3-[(1R)-2,2-difluoro-1-[4-[[1-(trifluoromethyl)cyclopropyl]methoxy]-2-pyridyl]ethoxy]-1-methyl-pyrazolo[3,4-c]pyridazin-5-yl]-1H-pyrimidine-2,4-dione;
5-[3-[(1R)-2,2-difluoro-1-[1-(2,2,2-trifluoroethyl)indazol-6-yl]ethoxy]-1-methyl-pyrazolo[3,4-c]pyridazin-5-yl]-1H-pyrimidine-2,4-dione;
5-[3-[(1R)-2,2-difluoro-1-[4-(2,2,2-trifluoroethyl)-2,3-dihydro-1,4-benzoxazin-6-yl]ethoxy]-1-methyl-pyrazolo[3,4-c]pyridazin-5-yl]-1H-pyrimidine-2,4-dione;
5-[3-[(1R)-2,2-difluoro-1-[2-[[(1S)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione;
5-[3-[(1R)-2,2-difluoro-1-[2-[[(1R)-2,2,2-trifluoro-1-methyl-ethyl]amino]-4-pyridyl]ethoxy]-1-methyl-pyrazolo[3,4-c]pyridazin-5-yl]-1H-pyrimidine-2,4-dione;
5-[3-[(1R)-2,2-difluoro-1-[2-(2-methoxyethoxy)-4-pyridyl]ethoxy]-1-methyl-pyrazolo[3,4-c]pyridazin-5-yl]-1H-pyrimidine-2,4-dione;
5-[3-[(1R)-1-(2-ethoxy-4-pyridyl)-2,2-difluoro-ethoxy]-1-methyl-pyrazolo[3,4-c]pyridazin-5-yl]-1H-pyrimidine-2,4-dione;
5-[3-[(1R)-2,2-difluoro-1-[2-(1,1,2,2,2-pentadeuterioethoxy)-4-pyridyl]ethoxy]-1-methyl-pyrazolo[3,4-c]pyridazin-5-yl]-1H-pyrimidine-2,4-dione;
5-[3-[(1R)-1-[2-(4,4-difluoro-1-piperidyl)-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(4,4-difluorocyclohexyl)amino]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(3,3-difluoropyrrolidin-1-yl)methyl]-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[2-(2,2,2-trifluoroethoxymethyl)-4-pyridyl]ethoxy]-1-methyl-pyrazolo[3,4-c]pyridazin-5-yl]-1H-pyrimidine-2,4-dione;
5-[3-[(1R)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione;
5-[3-[(1S)-2,2-difluoro-1-[6-(2,2,2-trifluoroethoxy)pyrimidin-4-yl]ethoxy]-1-methyl-pyrazolo[3,4-c]pyridazin-5-yl]-1H-pyrimidine-2,4-dione;
5-[3-[(1R)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione;
5-[3-[(1S)-2,2-difluoro-1-[4-(trifluoromethoxymethyl)-2-pyridyl]ethoxy]-1-methyl-pyrazolo[3,4-c]pyridazin-5-yl]-1H-pyrimidine-2,4-dione;
5-[3-[(1R)-1-[2-[(1S)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-[(1R)-1-(cyclopropoxy)ethyl]-4-pyridyl]-2,2-difluoro-ethoxy]-1-methyl-pyrazolo[3,4-c]pyridazin-5-yl]-1H-pyrimidine-2,4-dione;
5-[3-[(1R)-1-[2-(3,3-difluorocyclobutyl)-4-pyridyl]-2,2-difluoro-ethoxy]-1-methyl-pyrazolo[3,4-c]pyridazin-5-yl]-1H-pyrimidine-2,4-dione;
5-[3-[(1R)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione;
5-[3-[(1S)-2,2-difluoro-1-[6-(1,1,2,2,2-pentadeuterioethoxy)pyrimidin-4-yl]ethoxy]-1-methyl-pyrazolo[3,4-c]pyridazin-5-yl]-1H-pyrimidine-2,4-dione;
5-[1-methyl-3-[(1R)-2,2-difluoro-1-[2-[(trans)-4-methyltetrahydrofuran-3-yl]oxy-4-pyridyl]ethoxy]pyrazolo[3,4-c]pyridazin-5-yl]-1H-pyrimidine-2,4-dione;
5-[3-[(1R)-2,2-difluoro-1-[2-[(1-methoxycyclopropyl)methoxy]-4-pyridyl]ethoxy]-1-methyl-pyrazolo[3,4-c]pyridazin-5-yl]-1H-pyrimidine-2,4-dione;
5-[3-[(1R)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione; and
5-[3-[(1S)-1-[6-(3,3-difluorocyclobutoxy)pyrimidin-4-yl]-2,2-difluoro-ethoxy]-1-methyl-pyrazolo[3,4-c]pyridazin-5-yl]-1H-pyrimidine-2,4-dione;
or a pharmaceutically acceptable salt thereof.

15. A process for the preparation of a compound according to claim 1, the process comprising any of the following steps:
a) Deprotection of a compound of formula (X), 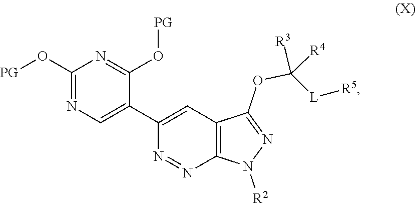
in the presence of an acid, dealkylation reagent, or metal mediated hydrogenation to afford the compound of formula (Ia), 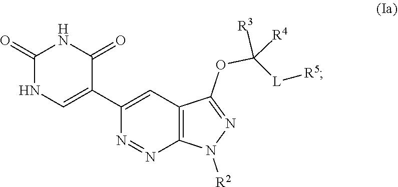
b) Deprotection of a compound of formula (XIII), 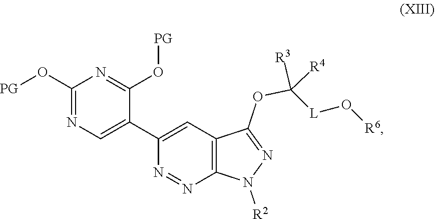
in the presence of an acid, dealkylation reagent, or metal mediated hydrogenation to afford the compound of formula (Ib), 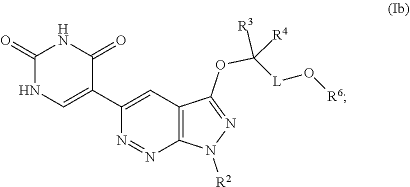
c) Stille coupling of a compound of formula (XV), 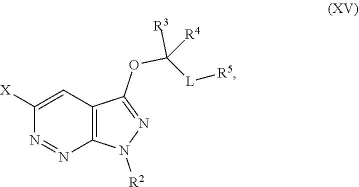
with organostannane (XVI), 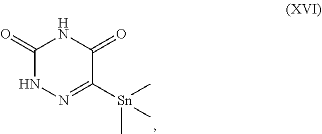
in the presence of a palladium catalyst to afford the compound of formula (Ic), 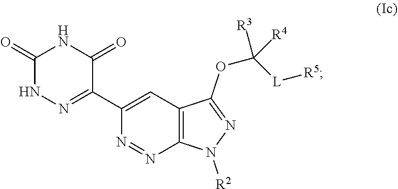
wherein
X is halogen; each PG is independently an oxygen protecting group; wherein PG is selected from methyl, tert-butyl, TBS, ethoxymethyl and benzyl; R⁶ is optionally substituted aryl, heteroaryl, heterocyclyl, arylC₁₋₆alkyl, heterocyclylC₁₋₆alkyl or heteroarylC₁₋₆alkyl;
in step a) and b), the acid is trifluoroacetic acid or aqueous hydrochloric acid; the dealkylation reagent is TMSCI and NaI; the hydrogenation is conducted with Pd/C; and
in step c), the catalyst is Pd(PPh₃)₄.

16. (canceled)

17. A pharmaceutical composition comprising a compound in accordance with claim 1 and a pharmaceutically acceptable excipient.

18. A method of treating cancer in a subject in need thereof, the method comprising administering a therapeutically effective amount of a compound according to claim 1.

19. The method according to claim 18, wherein the cancer is pancreatic cancer, colorectal cancer, gastric cancer, esophageal cancer, liver cancer, lung cancer, breast cancer, ovarian cancer, prostate cancer or melanoma.

20. The method according to claim 18 for inhibiting CD73.

21. (canceled)

22. (canceled)

23. A compound or pharmaceutically acceptable salt, when manufactured according to the process of claim 15.

24. (canceled)
